(19) United States
(12) Patent Application Publication
(10) Pub. No.: US 2004/0067950 A1
(43) Pub. Date: Apr. 8, 2004

(54) High affinity ligands for nociceptin receptor ORL-1

(75) Inventors: Deen Tulshian, Lebanon, NJ (US); Ginny D. Ho, Murray Hill, NJ (US); Lisa S. Silverman, Edison, NJ (US); Julius J. Matasi, Scotch Plains, NJ (US); Robbie L. McLeod, Branchburg, NJ (US); John A. Hey, Bloomfield, NJ (US); Richard W. Chapman, Somerville, NJ (US); Ana Bercovici, West Orange, NJ (US); Francis M. Cuss, Basking Ridge, NJ (US)

Correspondence Address: LERNER, DAVID, LITTENBERG,, KRUMHOLZ & MENTLIK, 600 SOUTH AVENUE WEST, WESTFIELD, NJ, 07090, US

(73) Assignee: Schering-Plough Corporation

(21) Appl. No.: 10/464,580

(22) Filed: Jun. 17, 2003

Related U.S. Application Data
(63) Continuation of application No. 09/491,780, filed on Jan. 26, 2000.
Application No. 09/491,780 is a continuation-in-part of application No. 09/359,771, filed on Jul. 26, 1999, now Pat. No. 6,262,066.
(60) Provisional application No. 60/094,240, filed on Jul. 27, 1998.

(51) Int. Cl. A61K 31/517; A61K 31/498; A61K 31/4439; C 07D 4 3/02

(52) U.S. Cl. 514/249; 514/266.22; 514/338; 544/284; 544/354; 546/268.4; 546/273.7

(57) ABSTRACT

The present invention relates to the method of treating cough with ORL-1 agonists, alone or in combination with additional agents for treating symptoms of cough, allergy or asthma, pharmaceutical compositions comprising the combinations, and to compounds of the formula

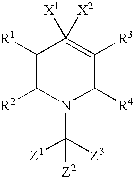

or a pharmaceutically acceptable salt or solvate thereof, wherein:

the dotted line represents an optional double bond;

X¹ is optionally substituted alkyl, cycloalkyl, aryl, heteroaryl or heterocycloalkyl;

X² is —CHO, —CN, optionally substituted amino, alkyl, or aryl;

or X¹ is optionally substituted benzofused heterocyclyl and X² is hydrogen;

or X¹ and X² together form an optionally benzofused spiro heterocyclyl group R¹, R², R³ and R⁴ are independently H and alkyl, or (R¹ and R⁴) or (R² and R³) or (R¹ and R³) or (R² and R⁴) together can form an alkylene bridge of 1 to 3 carbon atoms;

Z¹ is optionally substituted alkyl, aryl, heteroaryl, cycloalkyl or heterocycloalkyl, or —CO₂(alkyl or substituted amino) or CN ; Z² is H or Z¹; Z³ is H or alkyl; or Z¹, Z² and Z³, together with the carbon to which they are attached, form bicyclic saturated or unsaturated rings;

pharmaceutical compositions therefore, and the use of said compounds as nociceptin receptor inhibitors useful in the treatment of pain, anxiety, cough, asthma, depression and alcohol abuse.

### CROSS REFERENCE TO RELATED APPLICATIONS

This is a continuation of U.S. Ser. No. 09/491,780, filed Jan. 26, 2000, which is a continuation-in-part of U.S. Ser. No. 09/359,771, filed Jul. 26, 1999, which claims the benefit of U.S. Provisional Application 60/094,240, filed Jul. 27, 1998.

### BACKGROUND

The nociceptin receptor ORL-1 has been shown to be involved with modulation of pain in animal models. ORL-1 (the nociceptin receptor) was discovered as an “orphan opioid-like receptor” i.e. a receptor whose ligand was unknown. The nociceptin receptor is a G protein coupled receptor. While highly related in structure to the three classical opioid receptors, i.e. the targets for traditional opioid analgesics, it is not activated by endogenous opioids. Similarly, endogenous opioids fail to activate the nociceptin receptor. Like the classical opioid receptors, the nociceptin receptor has a broad distribution in the central nervous system.

In late 1995, nociceptin, also known as Orphanin FQ (OFQ), was discovered and shown to be an endogenous peptide ligand that activates the nociceptin receptor. Data included in the initial publications suggested that nociceptin and its receptor are part of a newly discovered pathway involved in the perception of painful stimuli. Subsequent work from a number of laboratories has shown that nociceptin, when administered intraspinally to rodents, is an analgesic. The efficacy of nociceptin is similar to that of endogenous opioid peptides. Recent data has shown that nociceptin acts as an anxiolytic when administered directly into the brain of rodents. When tested in standard animals models of anxiety, the efficacy of nociceptin is similar to that seen with classical benzodiazapine anxiolytics. These data suggest that a small molecule agonist of the nociceptin receptor could have significant analgesic or anxiolytic activity.

Additional recent data (Rizzi, et al, Life Sci., 64, (1999), p. 157-163) has shown that the activation of nociceptin receptors in isolated guinea pig bronchus inhibits tachykinergic, non-adrenergic, non-cholinergic contractions, indicating that nociceptin receptor agonists could be useful in the treatment of asthma. Also, it has been reported (Ciccocioppo et al, Physchopharmacology, 141 (1999), p. 220-224) nociceptin reduces the rewarding properties of ethanol in msP alcohol preferring rats, suggesting that intervention of nociceptin could be useful in the treatment of alcohol abuse. In EP 856,514, 8-substituted 1,3,8-triazaspiro[4,5]decan-4-on derivatives were disclosed as agonists and/or antagonists of orphanin FQ (i.e., nociceptin) useful in the treatment of various disorders, including depression; 2-oxoimidazole derivatives disclosed in WO98/54168 were described as having similar utility. Earlier, benzimidazolyl piperidines were disclosed in U.S. Pat. No. 3,318,900 as having analgesic activity.

Potent analgesic agents such as traditional opioids, e.g. morphine, carry with them significant side-effects. Clinically relevant side-effects include tolerance, physical dependence, respiratory depression, sedation and a decrease in gastrointestinal motility. For many patients, particularly those subjected to chronic opioid therapy, i.e. cancer patients, these side effects limit the dose of opioid that can be administered. Clinical data suggests that more than one-third of cancer patients have pain which is poorly controlled by present agents. Data obtained with nociceptin suggest the potential for advantages over opioids. When administered chronically to rodents, nociceptin, in contrast to morphine, showed no addiction liability. Additionally, chronic morphine treatment did not lead to a “cross-tolerance” to nociceptin, suggesting that these agents act via distinct pathways.

In view of the current interest in pain relief, a welcome contribution to the art would be additional compounds useful for modifying the effect of nociceptin, a natural ligand to ORL-1 and therefore useful in the management of pain and anxiety. Such a contribution is provided by this invention. We have also unexpectedly found that ORL-1 receptor agonists are useful in treating cough.

### SUMMARY OF THE INVENTION

Compounds of the present invention are represented by formula I

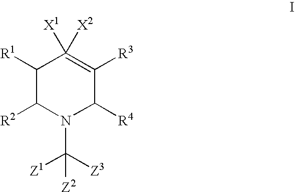

or a pharmaceutically acceptable salt or solvate thereof, wherein:

the dotted line represents an optional double bond;

X¹ is R⁵—(C₁-C₁₂)alkyl, R⁶—(C₃-C₁₂)cycloalkyl, R⁷-aryl, R⁸-heteroaryl or R¹⁰—(C₃-C₇)heterocycloalkyl;

X² is —CHO, —CN, —NHC(═NR²⁶)NHR²⁶, —CH(═NOR²⁶), —NHOR²⁶, R⁷-aryl, R⁷-aryl(C₁-C₆)alkyl, R⁷-aryl(C₁-C₆)alkenyl, R⁷-aryl(C₁-C₆)-alkynyl, —(CH₂)ᵥOR¹³, —(CH₂)ᵥCOOR²⁷, —(CH₂)ᵥCONR¹⁴R¹⁵, —(CH₂)ᵥNR²¹R²² or —(CH₂)ᵥNHC(O)R²¹, wherein v is zero, 1, 2 or 3 and wherein q is 1 to 3 and a is 1 or 2;

or X¹ is

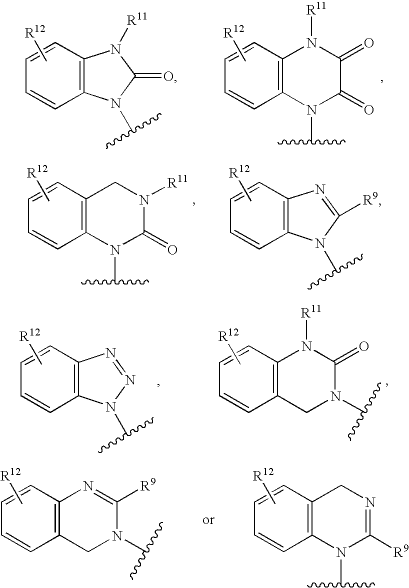

and X² is hydrogen;

or X¹ and X² together form a spiro group of the formula

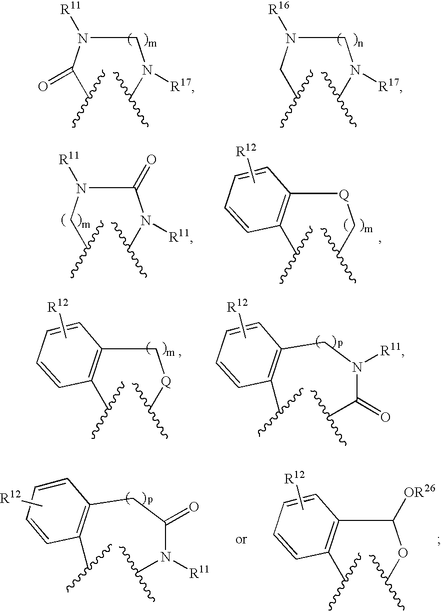

is 1 or2;

n is 1, 2 or 3, provided that when n is 1, one of R¹⁶ and R¹⁷ is —C(O)R²⁸;

p is 0 or 1;

Q is —CH₂—, —O—, —S—, —SO—, —SO₂— or —NR¹⁷—;

R¹, R², R³ and R⁴ are independently selected from the group consisting of hydrogen and (C₁-C₆)alkyl, or (R¹ and R⁴) or (R² and R³) or (R¹ and R³) or (R² and R⁴) together can form an alkylene bridge of 1 to 3 carbon atoms;

R⁵ is 1 to 3 substituents independently selected from the group consisting of H, R⁷-aryl, R⁶—(C₃-C₁₂)cycloalkyl, R⁸-heteroaryl, R¹⁰—(C₃-C₇)heterocycloalkyl, —NR¹⁹R²⁰, —OR¹³ and —S(O)₀₋₂R¹³;

R⁶ is 1 to 3 substituents independently selected from the group consisting of H, (C₁-C₆)alkyl, R⁷-aryl, —NR¹⁹R²⁰, —OR¹³ and —SR¹³;

R⁷ is 1 to 3 substituents independently selected from the group consisting of hydrogen, halo, (C₁-C₆)alkyl, R²⁵-aryl, (C₃-C₁₂)cycloalkyl, —CN, —CF₃, —OR¹⁹, —(C₁-C₆)alkyl-OR¹⁹, —OCF₃, —NR¹⁹R²⁰, —(C₁ -C₆)alkyl-NR¹⁹R²⁰, —NHSO₂R¹⁹, —SO₂N(R²⁶)₂, —SO₂R¹⁹, —SOR¹⁹, —SR¹⁹, —NO₂, —CONR¹⁹R²⁰, —NR²⁰COR¹⁹, —COR¹⁹, —COCF₃, —OCOR¹⁹, —OCO₂R¹⁹, —COOR¹⁹, —(C₁-C₆)alkyl-NHCOOC(CH₃)₃, —(C₁-C₆)alkyl-NHCOCF₃, —(C₁-C₆)alkyl-NHSO₂—(C₁-C₆)alkyl, —(C₁-C₆)alkyl-NHCONH—(C₁-C₆)-alkyl or

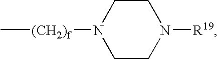

wherein f is 0 to 6; or R⁷ substituents on adjacent ring carbon atoms may together form a methylenedioxy or ethylenedioxy ring;

R⁸ is 1 to 3 substituents independently selected from the group consisting of hydrogen, halo, (C₁-C₆)alkyl, R²⁵-aryl, (C₃-C₁₂)cycloalkyl, —CN, —CF₃, —OR¹⁹, —(C₁-C₆)alkyl-OR¹⁹, —OCF₃, —NR¹⁹R²⁰, —(C₁-C₆)alkyl-NR¹⁹R²⁰, —NHSO₂R¹⁹, —SO₂N(R²⁶)₂, —NO₂, —CONR¹⁹R²⁰, —NR²⁰COR¹⁹, —COR¹⁹, —OCOR¹⁹, —OCO₂R¹⁹ and —COOR¹⁹;

R⁹ is hydrogen, (C₁-C₆)alkyl, halo, —OR¹⁹, —NR¹⁹R²⁰, —NHCN, —SR¹⁹ or —(C₁-C₆)alkyl-NR¹⁹R²⁰;

R¹⁰ is H, (C₁-C₆)alkyl, —OR¹⁹, —(C₁-C₆)alkyl-OR¹⁹, —NR¹⁹R²⁰ or —(C₁-C₆)alkyl-NR¹⁹R²⁰;

R¹¹ is independently selected from the group consisting of H, R⁵—(C₁-C₆)alkyl, R⁶—(C₃-C₁₂)cycloalkyl, —(C₁-C₆)alkyl(C₃-C₁₂)cycloalkyl, —(C₁-C₆)alkyl-OR¹⁹, —(C₁-C₆)alkyl-NR¹⁹R²⁰ and

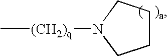

wherein q and a are as defined above;

R¹² is H, (C₁-C₆)alkyl, halo, —NO₂, —CF₃, —OCF₃, —OR¹⁹, —(C₁-C₆)alkyl-OR¹⁹, —NR¹⁹R²⁰ or —(C₁-C₆)alkyl-NR¹⁹R²⁰;

R¹³ is H, (C₁-C₆)alkyl, R⁷-aryl, —(C₁-C₆)alkyl-OR¹⁹, —(C₁-C₆)alkyl-NR¹⁹R²⁰; —(C₁-C₆)alkyl-SR¹⁹; or aryl (C₁-C₆) alkyl;

R¹⁴ and R¹⁵ are independently selected from the group consisting of H, R⁵—(C₁-C₆)alkyl, R⁷-aryl and

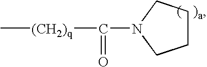

wherein q and a are as defined above;

R¹⁶ and R¹⁷ are independently selected from the group consisting of hydrogen, R⁵—(C₁-C₆)alkyl, R⁷-aryl, (C₃-C₁₂)cycloalkyl, R⁸-heteroaryl, R⁸-heteroaryl (C₁-C₆)alkyl, —C(O)R²⁸, —(C₁-C₆)alkyl(C₃-C₇)-heterocycloalkyl, —(C₁-C₆)alkyl-OR¹⁹ and —(C₁-C₆)alkyl-SR¹⁹;

R¹⁹ and R²⁰ are independently selected from the group consisting of hydrogen, (C₁-C₆)alkyl, (C₃-C₁₂)cycloalkyl, aryl and aryl(C₁-C₆)alkyl;

R²¹ and R²² are independently selected from the group consisting of hydrogen, (C₁-C₆)alkyl, (C₃-C₁₂)cycloalkyl, (C₃-C₁₂)cycloalkyl(C₁-C₆)alkyl, (C₃-C₇)heterocycloalkyl, —(C₁-C₆)alkyl(C₃-C₇)-heterocycloalkyl, R⁷-aryl, R⁷-aryl(C₁-C₆)alkyl, R⁸-heteroaryl(C₁-C₁₂)alkyl, —(C₁-C₆)alkyl-OR¹⁹, —(C₁-C₆)alkyl-NR¹⁹R²⁰, —(C₁-C₆)alkyl-SR¹⁹, —(C₁-C₆)alkyl-NR¹⁸—(C₁-C₆)alkyl-O—(C₁-C₆)alkyl-NR¹⁸—(C₁-C₆)alkyl-NR¹⁸—(C₁-C₆)alkyl;

R¹⁸ is hydrogen or (C₁-C₆)alkyl;

Z¹ is R⁵—(C₁-C₁₂)alkyl, R⁷-aryl, R⁸-heteroaryl, R⁶—(C₃-C₁₂)cyclo-alkyl, R¹⁰—(C₃-C₇)heterocycloalkyl, —CO₂(C₁-C₆)alkyl, CN or —C(O)NR¹⁹R²⁰; Z² is hydrogen or Z¹; Z³ is hydrogen or (C₁-C₆)alkyl; or Z¹, Z² and Z³, together with the carbon to which they are attached, form the group

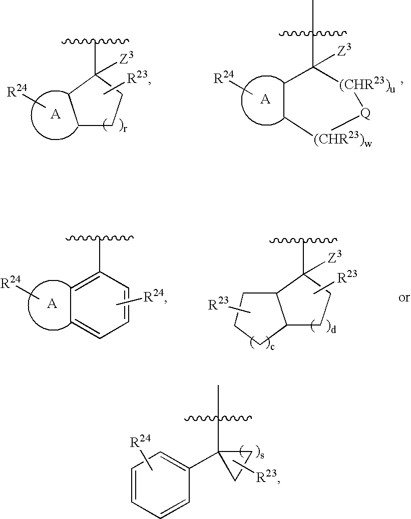

wherein r is 0 to 3; w and u are each 0-3, provided that the sum of w and u is 1-3; c and d are independently 1 or 2; s is 1 to 5; and ring A is a fused R⁷-phenyl or R⁸-heteroaryl ring;

R²³ is 1 to 3 substituents independently selected from the group consisting of H, (C₁-C₆)alkyl, —OR¹⁹, —(C₁-C₆)alkyl-OR¹⁹, —NR¹⁹R²⁰ and —(C₁-C₆)alkyl-NR¹⁹R²⁰;

R²⁴ is 1 to 3 substituents independently selected from the group consisting of R²³, —CF₃, —OCF₃, NO₂ or halo, or R²⁴ substituents on adjacent ring carbon atoms may together form a methylenedioxy or ethylenedioxy ring;

R²⁵ is 1-3 substituents independently selected from the group consisting of H, (C₁-C₆)alkyl, (C₁-C₆)alkoxy and halo;

R²⁶ is independently selected from the group consisting of H, (C₁-C₆)alkyl and R²⁵—C₆H₄—CH₂—;

R²⁷ is H, (C₁-C₆)alkyl, R⁷-aryl(C₁-C₆)alkyl, or (C₃-C₁₂)cycloalkyl;

R²⁸ is (C₁-C₆)alkyl, —(C₁-C₆)alkyl(C₃-C₁₂)cycloalkyl, R⁷-aryl, R⁷-aryl-(C₁-C₆)alkyl, R⁸-heteroaryl, —(C₁-C₆)alkyl-NR¹⁹R²⁰, —(C₁-C₆)alkyl-OR¹⁹ or —(C₁-C₆)alkyl-SR¹⁹;

provided that when X¹ is

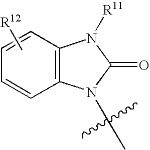

or X¹ and X² together are

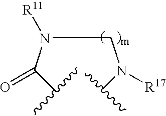

and Z¹ is R⁷-phenyl, Z² is not hydrogen or (C₁-C₃)alkyl;

provided that when Z¹, Z² and Z³, together with the carbon to which they are attached, form

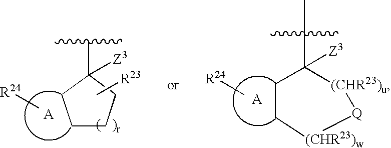

and X¹ and X² together are

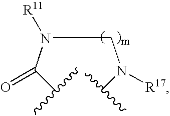

R¹¹ is not H, (C₁-C₆)alkyl, (C₁-C₆)alkoxy(C₁-C₆)alkyl or (C₁-C₆)hydroxyalkyl;

provided that when R² and R⁴ form an alkylene bridge, Z¹, Z² and Z³, together with the carbon to which they are attached, are not

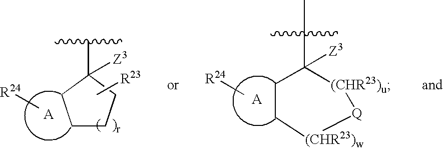

provided that when X¹ is

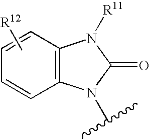

and Z¹ is R⁶—(C₃-C₁₂)-cycloalkyl, Z² is not H.

Preferred compounds of the invention are those wherein Z¹ and Z² are each R⁷-aryl, particularly R⁷-phenyl. Preferred R⁷ substituents are (C₁-C₆)alkyl and halo, with ortho-substitution being more preferred.

Compounds wherein R¹, R², R³ and R⁴ are each hydrogen are preferred, as well as compounds wherein R¹ and R³ are each hydrogen and R² and R⁴ are an alkylene bridge of 2 or 3 carbons.

Preferred are compounds wherein X¹ is R⁷-aryl, for example R⁷-phenyl, and X² is OH (i.e., X² is —(CH₂)ᵥOR¹³, wherein v is 0 and R¹³ is H) or —NC(O)R²⁸, compounds wherein X¹ is

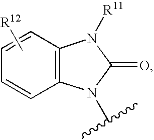

wherein R¹² is hydrogen and R¹¹ is (C₁-C₆)alkyl, —(C₁-C₆) alkyl(C₃-C₁₂)cycloalkyl, —(C₁-C₆)alkyl-OR¹⁹ or —(C₁-C₆)alkyl-NR¹⁹R²⁰; and compounds wherein X¹ and X² together form the spirocyclic group

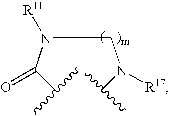

wherein m is 1, R¹⁷ is phenyl and R¹¹ is —(C₁-C₆)alkyl-OR¹⁹ or —(C₁-C₆)alkyl-NR¹⁹R², or

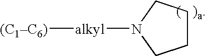

Also preferred are compounds of formula I wherein X¹ and X² form a spiro group, Z¹ is R⁷-aryl, preferably R⁷-phenyl, and Z² is C₄₋₁₂ alkyl.

In another aspect, the invention relates to a pharmaceutical composition comprising a compound of formula I and a pharmaceutically acceptable carrier. The compounds of the present invention are agonists and/or antagonists of the ORL-1 receptor, and therefore, in another aspect, the invention relates to a method of treating pain, anxiety, asthma, alcohol abuse or depression, comprising administering to a mammal in need of such treatment an effective amount of a compound of formula I.

In another aspect, the invention relates to a method of treating cough comprising administering an ORL-1 agonist to a mammal in need of such treatment. The use of any compound having ORL-1 agonist activity is claimed, but the following non-limiting list of compounds exemplifies ORL-1 agonists:

a) a compound represented by the structural formula IA:

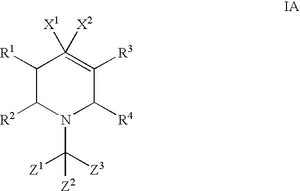

or a pharmaceutically acceptable salt or solvate thereof, wherein:

the dotted line represents an optional double bond;

X¹ is R⁵—(C₁-C₁₂)alkyl, R⁶—(C₃-C₁₂)cycloalkyl, R⁷-aryl, R⁸-heteroaryl or R¹⁰—(C₃-C₇)heterocycloalkyl;

X² is —CHO, —CN, —NHC(═NR²⁶)NHR²⁶, —CH(═NOR²⁶), —NHOR²⁶, R⁷-aryl, R⁷-aryl(C₁-C₆)alkyl, R⁷-aryl(C₁-C₆)alkenyl, R⁷-aryl(C₁-C₆)-alkynyl, —(CH₂)ᵥOR¹³, —(CH₂)ᵥCOOR²⁷, —(CH₂)ᵥCONR¹⁴R¹⁵, —(CH₂)ᵥNR²¹R²² or —(CH₂)ᵥNHC(O)R²¹, wherein v is zero, 1, 2 or 3 and wherein q is 1 to 3 and a is 1 or 2;

or X¹ is

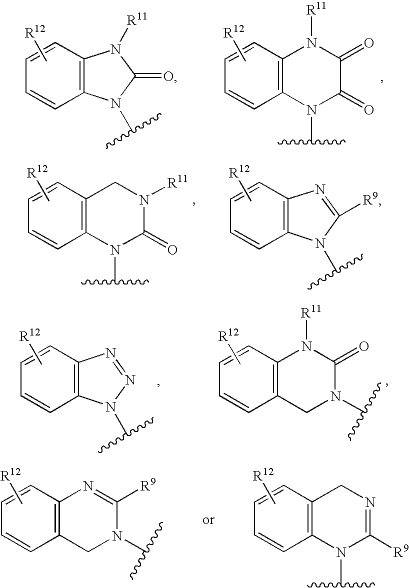

and X² is hydrogen;

or X¹ and X² together form a spiro group of the formula

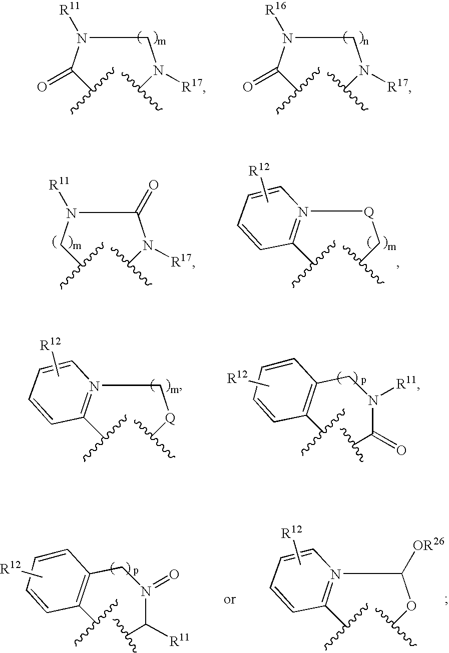

is 1 or 2;

n is 1, 2 or 3, provided that when n is 1, one of R¹⁶ and R¹⁷ is —C(O)R²⁸;

p is 0 or 1;

Q is —CH₂—, —O—, —S—, —SO—, —SO₂—or —NR¹⁷—;

R¹, R², R³ and R⁴ are independently selected from the group consisting of hydrogen and (C₁-C₆)alkyl, or (R¹ and R⁴) or (R² and R³) or (R¹ and R³) or (R² and R⁴) together can form an alkylene bridge of 1 to 3 carbon atoms;

R⁵ is 1 to 3 substituents independently selected from the group consisting of H, R⁷-aryl, R⁶—(C₃-C₁₂)cycloalkyl, R⁸-heteroaryl, R¹—(C₃-C₇)heterocycloalkyl, —NR¹⁹R²⁰, —OR¹³ and —S(O)₀₋₂R¹³;

R⁶ is 1 to 3 substituents independently selected from the group consisting of H, (C₁-C₆)alkyl, R⁷-aryl, —NR¹⁹R²⁰, —OR¹³ and —SR¹³;

R⁷ is 1 to 3 substituents independently selected from the group consisting of hydrogen, halo, (C₁-C₆)alkyl, R²⁵-aryl, (C₃-C₁₂)cycloalkyl, —CN, —CF₃, —OR¹⁹, —(C₁-C₆)alkyl-OR¹⁹, —OCF₃, —NR¹⁹R²⁰, —(C₁-C₆)alkyl-NR¹⁹R²⁰, —NHSO₂R¹⁹, —SO₂N(R²⁶)₂, —SO₂R¹⁹, —SOR¹⁹, —SR¹⁹, —NO₂, —CONR¹⁹R²⁰, —NR²⁰COR¹⁹, —COR¹⁹, —COCF₃, —OCOR¹⁹, —OCO₂R¹⁹, —COOR¹⁹, —(C₁-C₆)alkyl-NHCOOC(CH₃)₃, —(C₁-C₆)alkyl-NHCOCF₃, —(C₁-C₆)alkyl-NHSO₂—(C₁-C₆)alkyl, —(C₁-C₆)alkyl-NHCONH—(C₁-C₆)-alkyl or

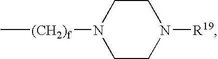

wherein f is 0 to 6; or R⁷ substituents on adjacent ring carbon atoms may together form a methylenedioxy or ethylenedioxy ring;

R⁸ is 1 to 3 substituents independently selected from the group consisting of hydrogen, halo, (C₁-C₆)alkyl, R²⁵-aryl, (C₃-C₁₂)cycloalkyl, —CN, —CF₃, —OR¹⁹, —(C₁-C₆)alkyl-OR¹⁹, —OCF₃, —NR¹⁹R²⁰, —(C₁-C₆)alkyl-NR¹⁹R²⁰, —NHSO₂R¹⁹, —SO₂N(R²⁶)₂, —NO₂, —CONR¹⁹R²⁰, —NR²⁰COR¹⁹, —COR¹⁹, —OCOR¹⁹, —OCO₂R¹⁹ and —COOR¹⁹;

R⁹ is hydrogen, (C₁-C₆)alkyl, halo, —OR¹⁹, —NR¹⁹R²⁰, —NHCN, —SR¹⁹ or —(C₁-C₆)alkyl-NR¹⁹R²⁰;

R¹⁰ is H, (C₁-C₆)alkyl, —OR¹⁹, —(C₁-C₆)alkyl-OR¹⁹, —NR¹⁹R²⁰ or —(C₁-C₆)alkyl-NR¹⁹R²⁰;

R¹¹ is independently selected from the group consisting of H, R⁵—(C₁-C₆)alkyl, R⁶—(C₃-C₁₂)cycloalkyl, —(C₁-C₆)alkyl(C₃-C₁₂)cycloalkyl, —(C₁-C₆)alkyl-OR¹⁹, —(C₁-C₆)alkyl-NR¹⁹R²⁰ and

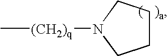

wherein q and a are as defined above;

R¹² is H, (C₁-C₆)alkyl, halo, —NO₂, —CF₃, —OCF₃, —OR¹⁹, —(C₁-C₆)alkyl-OR¹⁹, —NR¹⁹R²⁰ or —(C₁-C₆)alkyl-NR¹⁹R²⁰;

R¹³ is H, (C₁-C₆)alkyl, R⁷-aryl, —(C₁-C₆)alkyl-OR¹⁹, —(C₁-C₆)alkyl-NR¹⁹R²⁰; —(C₁-C₆)alkyl-SR¹⁹; or aryl (C₁-C₆) alkyl;

R¹⁴ and R¹⁵ are independently selected from the group consisting of H, R⁵—(C₁-C₆)alkyl, R⁷-aryl and

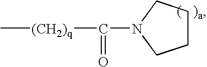

wherein q and a are as defined above;

R¹⁶ and R¹⁷ are independently selected from the group consisting of hydrogen, R⁵—(C₁-C₆)alkyl, R⁷-aryl, (C₃-C₁₂)cycloalkyl, R⁸-heteroaryl, R⁸-heteroaryl(C₁-C₆)alkyl, —C(O)R²⁸, —(C₁-C₆)alkyl(C₃-C₇)-heterocycloalkyl, —(C₁-C₆)alkyl-OR¹⁹ and —(C₁-C₆)alkyl-SR¹⁹;

R¹⁹ and R²⁰ are independently selected from the group consisting of hydrogen, (C₁-C₆)alkyl, (C₃-C₁₂)cycloalkyl, aryl and aryl(C₁-C₆)alkyl;

R²¹ and R²² are independently selected from the group consisting of hydrogen, (C₁-C₆)alkyl, (C₃-C₁₂)cycloalkyl, (C₃-C₁₂)cycloalkyl(C₁-C₆)alkyl, (C₃-C₇)heterocycloalkyl, —(C₁-C₆)alkyl(C₃-C₇)-heterocycloalkyl, R⁷-aryl, R⁷-aryl(C₁-C₆)alkyl, R⁸-heteroaryl(C₁-C₁₂)alkyl, —(C₁-C₆)alkyl-OR¹⁹, —(C₁-C₆)alkyl-NR¹⁹R²⁰, —(C₁-C₆)alkyl-SR¹⁹, —(C₁-C₆)alkyl-NR¹⁸—(C₁-C₆)alkyl-O—(C₁-C₆)alkyl and —(C₁-C₆)alkyl-NR¹⁸—(C₁-C₆)alkyl-NR¹⁸—(C₁-C₆)alkyl;

R¹⁸ is hydrogen or (C₁-C₆)alkyl;

Z¹ is R⁵—(C₁-C₁₂)alkyl, R⁷-aryl, R⁸-heteroaryl, R⁶—(C₃-C₁₂)cyclo-alkyl, R¹⁰—(C₃-C₇)heterocycloalkyl, —CO₂(C₁-C₆)alkyl, CN or —C(O)NR¹⁹R²⁰; Z² is hydrogen or Z^{1;} Z³ is hydrogen or (C₁-C₆)alkyl; or Z¹, Z² and Z³, together with the carbon to which they are attached, form the group

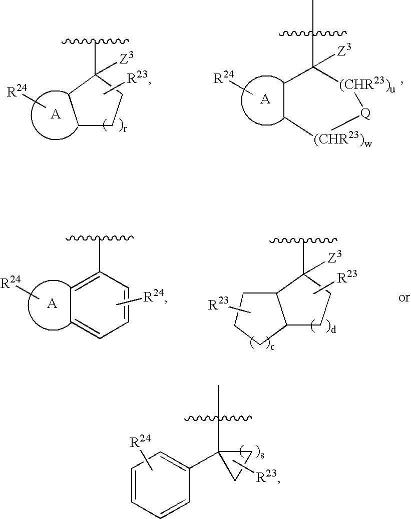

wherein r is 0 to 3; w and u are each 0-3, provided that the sum of w and u is 1-3; c and d are independently 1 or 2; s is 1 to 5; and ring A is a fused R⁷-phenyl or R⁸-heteroaryl ring;

R²³ is 1 to 3 substituents independently selected from the group consisting of H, (C₁-C₆)alkyl, —OR¹⁹, —(C₁-C₆)alkyl-OR¹⁹, —NR¹⁹R²⁰ and —(C₁-C₆)alkyl-NR¹⁹R²⁰;

R²⁴ is 1 to 3 substituents independently selected from the group consisting of R²³, —CF₃, —OCF₃, NO₂ or halo, or R²⁴ substituents on adjacent ring carbon atoms may together form a methylenedioxy or ethylenedioxy ring;

R²⁵ is 1-3 substituents independently selected from the group consisting of H, (C₁-C₆)alkyl, (C₁-C₆)alkoxy and halo;

R²⁶ is independently selected from the group consisting of H, (C₁-C₆)alkyl and R²⁵—C₆H₄—CH₂—;

R²⁷ is H, (C₁-C₆)alkyl, R⁷-aryl(C₁-C₆)alkyl, or (C₃-C₁₂)cycloalkyl; and

R²⁸ is (C₁-C₆)alkyl, —(C₁-C₆)alkyl(C₃-C₁₂)cycloalkyl, R⁷-aryl, R⁷-aryl-(C₁-C₆)alkyl, R⁸-heteroaryl, —(C₁-C₆)alkyl-NR¹⁹R²⁰, —(C₁-C₆)alkyl-OR¹⁹ or —(C₁-C₆)alkyl-SR¹⁹.

Preferred compounds of formula IA are the same as those for compounds of formula I.

b) a compound disclosed in EP 856,514 represented by the structural formula II:

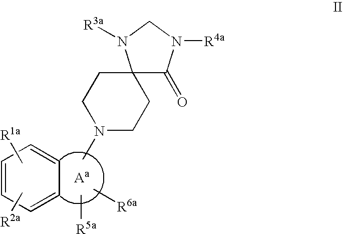

wherein

R^{1a} and R^{2a} are, independently from each other, hydrogen, lower alkyl, lower alkoxy or halogen;

R^{3a} is phenyl, optionally substituted by lower alkyl, CF₃, lower alkoxy or halogen; and

R^{4a} is hydrogen, lower alkyl, lower alkenyl, —C(O)-lower alkyl, —C(O)-phenyl, lower alkyl-C(O)-phenyl, lower alkylen-C(O)-lower alkyl, lower alkantriyl-di-C(O)O-lower alkyl, hydroxy-lower alkyl, lower alkyl-O-lower alkyl, lower alkyl-CH(OH)CF₃, phenyl or benzyl;

R^{5a} and R^{6a} are, independently from each other, hydrogen, phenyl, lower alkyl or di-lower alkyl or may form together a phenyl ring, and

R^{5a} and one of R^{1a} or R^{2a} may form together a saturated or unsaturated 6 membered ring,

A^{a} is a 4-7 membered saturated ring which may contain a heteroatom such as O or S,

or a pharmaceutically acceptable acid addition salt thereof.

Preferred compounds of formula II include:

(−)-8-(5,8-dichloro-1,2,3,4-tetrahydro-naphthyl-2)-1-phenyl-1,3,8-triaza-spiro[4.5]decan-4-one;

8-(8-chloro-1,2,3,4-tetrahydro-naphthyl-2)-1-phenyl-1,3,8-triaza-spiro[4.5]decan-4-one;

1-phenyl-8-(1,2,3,4-tetrahydro-naphthyl-1)-1,3,8-triaza-spiro[4.5]decan-4-one;

8-indan-2-yl-1-phenyl-1,3,8-triaza-spiro[4.5]decan-4-one;

(RS)-8-(acenaphthen-1-yl)-1-phenyl-1,3,8-triaza-spiro[4.5]decan-4-one;

(RS)-8-(acenaphthen-1-yl)-3-methyl-1-phenyl-1,3,8-triaza-spiro[4.5]decan-4-one;

(RS)-8-(2,3-dihydro-1H-phenalen-1-yl)-1-phenyl-1,3,8-triaza-spiro[4.5]decan-4-one;

(R)-8-(acenaphthen-1-yl)-1-phenyl-1,3,8-triaza-spiro[4.5]decan-4-one;

8-(2,3,3a,4,5,6-hexahydro-1H-phenalen-1-yl)-1-phenyl-1,3,8-triaza-spiro[4.5]decan-4-one; and

(RS)-8-(5-methyl-1,2,3,4-tetrahydro-naphthalen-1-yl)-1,3,8-triaza-spiro[4.5]decan-4-one.

c) a compound disclosed in EP 921,125 represented by the structural formula III

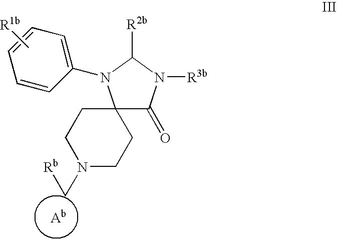

wherein

R^{1b} is hydrogen, lower alkyl, halogen, lower alkoxy, CF₃, lower alkyl-phenyl or (C₅₋₇)-cycloalkyl;

R^{2b} is hydrogen, lower alkyl, phenyl or lower alkyl-phenyl;

R^{3b} is hydrogen, lower alkyl, benzyl, lower alkyl-phenyl, lower alkyl-diphenyl, triazinyl, cyanomethyl, lower alkyl-piperidinyl, lower alkyl-naphthyl, (C₅₋₇)-cycloalkyl, lower alkyl-(C₅₋₇)-cycloalkyl, lower alkyl-pyridyl, lower alkyl-morpholinyl, lower alkyl dioxolanyl, lower alkyl, oxazolyl, or lower alkyl-2-oxo-oxazolidinyl and wherein the ring systems may be substituted by additional lower alkyl, lower alkoxy, CF₃ or phenyl, or —(CH₂)ₙC(O)O-lower alkyl, —(CH₂)ₙC(O)NH₂, —(CH₂)ₙC(O)N(lower alkyl)₂, —(CH₂)ₙOH or —(CH₂)ₙC(O)NHCH₂C₆H₆;

R^{4b} is hydrogen, lower alkyl or nitrilo;

A^{b} is a ring system, consisting of

(a) (C₅⁻₁₅)-cycloalkyl, which may be in addition to R^{4b} optionally substituted by lower alkyl, CF₃, phenyl, (C₅⁻₇)-cycloalkyl, spiro-undecan-alkyl or by 2-norbornyl, or is one of the following groups

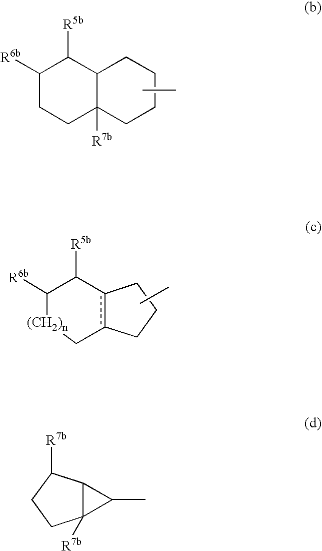

dodecahydro-acenaphthylen-1yl (e), bicyclo[6.2.0]dec-9-yl (f) and bicyclononan-9-yl (g); and wherein

R^{5b} and R^{6b} are hydrogen, lower alkyl, or taken together and with the carbon atoms to which they are attached form a phenyl ring;

R^{7b} is hydrogen or lower alkyl;

the dotted line represents an optional double bond and n is 1 to 4;

or a pharmaceutically acceptable acid addition salt thereof.

Preferred compounds of formula III include:

8-(decahydro-naphthalen-2-yl)-1-phenyl-1,3,8-triaza-spiro[4.5]decan-4-one;

8-(decahydro-naphthalen-2-yl)-3-methyl-1-phenyl-1,3,8-triaza-spiro[4.5]decan-4-one;

a mixture of (2RS,4aRS,8aSR)- and (2RS,4aRS,8aSR)-8-(decahydro-naphthalen-2-yl)-1-phenyl-1,3,8-triaza-spiro[4.5]decan-4-one;

(2RS,4aSR,8aRS)-8-(decahydro-naphthalen-2-yl)-1-phenyl-1,3,8-triaza-spiro[4.5]decan-4-one;

cis-8-(4-methyl-cyclohexyl)-1-phenyl-1,3,8-triaza-spiro[4.5]decan-4-one;

8-cyclodecyl-1-phenyl-1,3,8-triaza-spiro[4.5]decan-4-one;

8-cyclononyl-1-phenyl-1,3,8-triaza-spiro[4.5]decan-4-one;

cis-8-(4-isopropyl-cyclohexyl)-1-phenyl-1,3,8-triaza-spiro[4.5]decan-4-one;

(R,S)-8-cyclodecyl-1-(3-methyl-phenyl)-2-phenyl-1,3,8-triaza-spiro[4.5]decan-4-one;

8-cyclodecyl-4-oxo-1-phenyl-1,3,8-triaza-spiro[4.5]dec-3-yl)-acetonitrile;

8-(cis-octahydro-inden-2-yl)l-1-phenyl-1,3,8-triaza-spiro[4.5]decan-4-one; and

8-(cis-bicyclo[6.2.0]dec-9-yl)l-1-phenyl-1,3,8-triaza-spiro[4.5]decan-4-one.

d) a compound disclosed in WO 99/36421 represented by the structural formula IV:

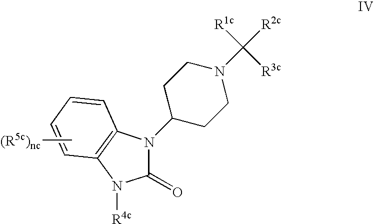

or a pharmaceutically acceptable salt thereof, wherein

R^{1c} and R^{2c} are independently C₁-C₄ alkyl; or

R^{1c} and R^{2c}, taken together with the carbon to which they are attached, form a mono-, bi-, tri- or spiro-cyclic group having 6 to 13 carbon atoms, wherein the cyclic group is optionally substituted by 1 to 5 substituents independently selected from C₁-C₄ alkyl, C₂-C₄ alkylene, C₁-C₄ alkoxy, hydroxy, oxo, ═CH₂ and ═CH—C₁-C₄ alkyl;

R^{3c} is C₁-C₇ alkyl, C₂-C₅ alkenyl, C₂-C₅ alkynyl, phenyl-C₁-C₅ alkyl, phenyl optionally substituted by 1 to 3 substituents independently selected from fluorine, C₁-C₃ alkyl and C₁-C₃ alkoxy, or a heteroaryl group selected from furyl, theinyl, pyrrolyl and pyridyl, wherein said heteroaryl group is optionally substituted by 1 to 3 substituents independently selected from halo, C₁-C₃ alkyl and C₁-C₃ alkoxy, with the proviso that when both R^{1c} and R^{2c} are C₁-C₄ alkyl, then R^{3c} is other than C₁-C₇ alkyl, C₂-C₅ alkenyl and C₂-C₅ alkynyl;

R^{4c} is selected from

1) hydrogen;

2) optionally substituted mono- or di-substituted C₁-C₈ alkyl, C₃-C₇ cycloalkyl, C₂-C₈ alkenyl, C₂-C₈ alkynyl, C₁C₆ alkyl-Z^{c}-, C₁C₆ alkyl-Z^{c}-(C₁C₆)alkyl, C₃-C₇ cycloalkyl-Z^{c}-(C₁C₆)alkyl, C₂-C₆ alkenyl-Z^{c}-(C₁C₆)alkyl or C₂-C₆ alkynyl-Z^{c}-(C₁C₆)alkyl, wherein Z^{c} is selected from O, S, SO, SO₂, CO, CO₂, OCO, NRC, CONRC and NR^{c}CO, wherein R^{c} is hydrogen or C₁-C₆ alkyl, and the substituents to be attached to the alkyl, alkenyl, alkynyl or cycloalkyl moiety are independently selected from halo, hydroxy, carboxy, amino, mono- or di-(C₁-C₄ alkyl)amino, hydrazino, azido, ureido, amidino and guanidino; or

3) optionally mono- or di-substituted aryl, heterocyclic, aryl(C₁-C₅)alkyl, heterocyclic(C₁-C₅)alkyl, heterocyclic-heterocyclic(C₁-C₅)alkyl, aryl-heterocyclic(C₁-C₅)alkyl, heterocyclic-Z^{c}-(C₁-C₅)alkyl, aryl-Z^{c}-(C₁-C₅)alkyl, aryl(C₁-C₅)alkyl-Z^{c}-(C₁-C₅)alkyl, or heterocyclic(C₁-C₅)alkyl-Z^{c}-(C₁-C₅)alkyl, wherein Z^{c} is selected from O, S, SO, SO₂, CO, CO₂, OCO, NRC, CONRC and NRCCO, wherein R^{c} is hydrogen or C₁-C₆ alkyl, and the substituents to be attached to the aryl or heterocyclic moiety are independently selected from halo, hydroxy, carboxy, C₁-C₄ alkyl, C₁-C₄ alkoxy, C₁-C₄ alkyl-CO—, amino(C₁-C₄)alkyl-CO—, phenyl, benzyl, amino, mono- or di-(C₁-C₄ alkyl)amino, hydrazino, azido, ureido, amidino and guanidino;

R^{5c} is independently selected from halo, C₁-C₃ alkyl, C₁-C₃ alkoxy, C₁-C₃ alkylsulfonyl, CF₃, carboxy, hydroxy, amino, alkylamino, acylamino, arylcarbonyl, alkylcarbonyl and hydroxyalkyl; and

n is 0, 1, 2, 3 or 4.

Preferred compounds of formula IV include:

1-{1-[1-methyl-1-(2-thienyl)ethyl]-4-piperidinyl}1,3-dihydro-2H-1,3-benzimidazol-2-one;

1-[1-(1-phenylcycloheptyl)-4-piperidinyl]-1,3-dihydro-2H-1,3-benzimidazol-2-one;

1-[4-piperidinyl-1-(1-propylcyclononyl)]-1,3-dihydro-2H-1,3-benzimidazol-2-one;

1-[1-(1-phenylcyclooctyl)-4-piperidinyl]-1,3-dihydro-2H-1 ,3-benzimidazol-2-one;

1-[1-(1-phenylcyclononyl)-4-piperidinyl]-1,3-dihydro-2H-1,3-benzimidazol-2-one;

1-{1-[1-(4-fluorophenyl)cyclohepyl]-4-piperidinyl}-1,3-d ihydro-2H-1,3-benzimidazol-2-one;

1-[1-(1-methylcyclononyl)-4-piperidinyl]-1,3-dihydro-2H-1,3-benzimidazol-2-one;

1-[1-(1-ethylcyclononyl)-4-piperidinyl]-1,3-dihydro-2H-1,3-benzimidazol-2-one;

1-[1-(1-methylcyclooctyl)-4-piperid inyl]-1,3-dihydro-2H- 1,3-benzimidazol-2-one;

1-[1-(1-phenylcyclohept-4-enyl)-4-piperidinyl]-1,3-dihydro-2H-1,3-benzimidazol-2-one;

1-(6-aminohexyl)-3-[1-(1-phenylcycloheptyl)-4-piperidinyl]-1,3-dihydro-2H-1,3-benzimidazol-2-one;

1-(2-aminoethyl)-3-[1-(1-phenylcyclohept-4-enyl)-4-piperidinyl]-1,3-dihydro-2H-1,3-benzimidazol-2-one; and

1-[1-(1-phenylcycloheptyl)-4-piperidinyl]-3-(2-piperidinoethyl)-1,3-dihydro-2H-1,3-benzimidazol-2-one.

e) a compound disclosed in WO 98/54168 represented by the structural formula V

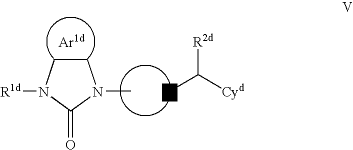

or a salt or ester thereof, wherein

Ar^{1d} is an optionally substituted aromatic carbon ring or heterocycle, wherein the optional substituents are independently selected from halo, alkyl, amino, alkylamino, dialkylamino, hydroxy, alkoxy and carboxyl;

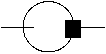

is an optionally substituted mono- or di-cyclic C₃₋₁₄ aliphatic nitrogenous heterocycle;

Cy^{d} is an optionally substituted mono-, di- or tri-cyclic C₃₋₂₀ aliphatic carbon ring;

R^{1d} is hydrogen, lower alkenyl, lower alkynyl, lower cycloalkyl, amino, lower alkylamino, di(lower alkyl)amino, hydroxy, lower alkoxy, carboxy, lower alkoxycarbonyl, carbamoyl, lower alkylcarbamoyl, di(lower alkyl)carbamoyl or optionally substituted lower alkyl; and

R^{2d} is hydrogen or lower alkyl.

f) a compound disclosed in WO 99/48492 represented by the structural formula VI

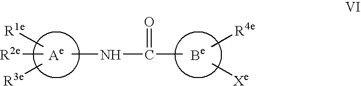

or a pharmaceutically acceptable salt thereof, wherein

A^{e} is an aryl or heterocyclyl ring;

B^{e} is phenyl, thienyl, furyl, pyrrolyl, pyrrolidinyl, oxazolyl or cyclohexenyl;

R^{1e} and R^{2e} are independently hydrogen, alkyl, hydroxyalkyl, amino, alkylamino or di-alkylamino;

R^{3e} and R^{4e} are independently hydrogen, halo or alkyl;

X^{e} is hydrogen, halo, alkyl, alkoxyalkyl, alkenyl, amino, CN, or —(CH₂)ₘₑ-E^{e}-(CH₂)ₙₑ-G^{e};

E^{e} is a bond, —CH═CR^{6e}, O, S, NR^{7e}, CO, SO₂ or NHCO;

G^{e} is aryl, heterocyclyl, cycloalkyl or fused aryl, all optionally substituted by 1-5 R^{5e} groups;

R^{5e} is independently selected from halo, OH, alkyl, alkyl optionally substituted by alkoxy, alkoxyalkoxy, halo, OH or alkanoyloxy, alkoxy, alkoxyalkoxy, amino, alkylamino, di-alkylamino, NO₂, CN, alkanoyl, alkanoyloxy, carboxy, alkoxycarbonyl, alkylsulfonyl and phenyl;

R^{6e} is hydrogen or aryl;

R^{7e} is hydrogen, alkyl or alkoxycarbonyl;

m^{e} is 0-8; and

n^{e} is 1-4.

A preferred compound of formula VI is:

N-(4-amino-2-methyl-6-quinolyl)-2-[(4-ethylphenoxy)methyl]benzamide.

The disclosures of EP 856,514, EP921,125, WO 99/36421, WO 98/54168 and WO 99/48492 are incorporated herein by reference.

In another aspect, the invention relates to a method of treating cough, comprising administering to a mammal in need of such treatment: (a) an effective amount of a nociceptin receptor ORL-1 agonist; and (b) an effective amount of one or more agents for treating cough, allergy or asthma symptoms selected from the group consisting of: antihistamines, 5-lipoxygenase inhibitors, leukotriene inhibitors, H₃ inhibitors, β-adrenergic receptor agonists, xanthine derivatives, α-adrenergic receptor agonists, mast cell stabilizers, anti-tussives, expectorants, decongestants, NK₁, NK₂ and NK₃ tachykinin receptor antagonists, and GABA_{B} agonists.

In still another aspect, the invention relates to a pharmaceutical composition comprising a nociceptin receptor ORL-1 agonist and one or more agents selected from the group consisting of: antihistamines, 5-lipoxygenase inhibitors, leukotriene inhibitors, H₃ inhibitors, β-adrenergic receptor agonists, xanthine derivatives, α-adrenergic receptor agonists, mast cell stabilizers, anti-tussives, expectorants, decongestants, NK₁, NK₂ and NK₃ tachykinin receptor antagonists, and GABA_{B} agonists.

Preferred ORL-1 agonists for use in the combination and in the combined pharmaceutical composition are those represented in formulas IA to VI. In yet another aspect, the present invention relates to a novel compound not included in the structure of formula I, said compound being:

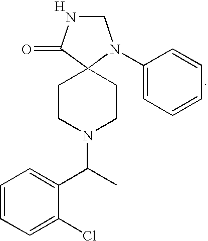

### BRIEF DESCRIPTION OF THE DRAWINGS

FIG. 1 illustrates the effect in guinea pigs of Compounds A and B (see Example 12) compared to baclofen on capsaicin-induced cough.

FIGS. 2A and 2B show changes in Tidal Volume after administration of Compound A or baclofen, and FIG. 2C shows changes in frequency of breaths after administration of Compound A or baclofen.

### DETAILED DESCRIPTION OF THE INVENTION

As used herein in formula I and IA, the following terms are used as defined below unless otherwise indicated:

M⁺ represents the molecular ion of the molecule in the mass spectrum and MH⁺ represents the molecular ion plus hydrogen of the molecule in the mass spectrum;

Bu is butyl; Et is ethyl; Me is methyl; and Ph is phenyl;

alkyl (including the alkyl portions of alkoxy, alkylamino and dialkylamino) represents straight and branched carbon chains containing from 1 to 12 carbon atoms or 1 to 6 carbon atoms; for example methyl, ethyl, propyl, iso-propyl, n-butyl, t-butyl, n-pentyl, isopentyl, hexyl and the like;

alkenyl represents an alkyl chain of 2 to 6 carbon atoms comprising one or two double bonds in the chain, e.g., vinyl, propenyl or butenyl;

alkynyl represents an alkyl chain of 2 to 6 carbon atoms comprising one triple bond in the chain, e.g., ethynyl or propynyl;

alkoxy represents an alkyl moiety covalently bonded to an adjacent structural element through an oxygen atom, for example, methoxy, ethoxy, propoxy, butoxy, pentoxy, hexoxy and the like;

aryl (including the aryl portion of arylalkyl) represents a carbocyclic group containing from 6 to 15 carbon atoms and having at least one aromatic ring (e.g., aryl is phenyl), wherein said aryl group optionally can be fused with aryl, (C₃-C₇)cycloalkyl, heteroaryl or hetero(C₃-C₇)cycloalkyl rings; and wherein R⁷-aryl means that any of the available substitutable carbon and nitrogen atoms in said aryl group and/or said fused ring(s) is optionally and independently substituted, and wherein the aryl ring is substituted with 1-3 R⁷ groups. Examples of aryl groups are phenyl, naphthyl and anthryl;

arylalkyl represents an alkyl group, as defined above, wherein one or more hydrogen atoms of the alkyl moiety have been substituted with one to three aryl groups; wherein aryl is as defined above;

aryloxy represents an aryl group, as defined above, wherein said aryl group is covalently bonded to an adjacent structural element through an oxygen atom, for example, phenoxy;

cycloalkyl represents saturated carbocyclic rings of from 3 to 12 carbon atoms, preferably 3 to 7 carbon atoms; wherein R⁶-cycloalkyl means that any of the available substitutable carbon atoms in said cycloalkyl group is optionally and independently substituted, and wherein the cycloalkyl ring is substituted with 1-3 R⁶ groups;

cycloalkylalkyl represents an alkyl group, as defined above, wherein one or more hydrogen atoms of the alkyl moiety have been substituted with one to three cycloalkyl groups, wherein cycloalkyl is as defined above;

halo represents fluoro, chloro, bromo and iodo;

heteroaryl represents cyclic groups having one to three heteroatoms selected from O, S and N, said heteroatom(s) interrupting a carbocyclic ring structure and having a sufficient number of delocalized pi electrons to provide aromatic character, with the aromatic heterocyclic groups containing from 5 to 14 carbon atoms, wherein said heteroaryl group optionally can be fused with one or more aryl, cycloalkyl, heteroaryl or heterocycloalkyl rings; and wherein any of the available substitutable carbon or nitrogen atoms in said heteroaryl group and/or said fused ring(s) may be optionally and independently substituted, and wherein the heteroaryl ring can be substituted with 1-3 R⁸ groups; representative heteroaryl groups can include, for example, furanyl, thienyl, imidazoyl, pyrimidinyl, triazolyl, 2-, 3- or 4-pyridyl or 2-, 3- or 4-pyridyl N-oxide wherein pyridyl N-oxide can be represented as:

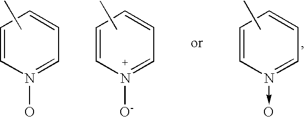

heteroarylalkyl represents an alkyl group, as defined above, wherein one or more hydrogen atoms have been replaced by one or more heteroaryl groups, as defined above;

heterocycloalkyl represents a saturated ring containing from 3 to 7 carbon atoms, preferably from 4 to 6 carbon atoms, interrupted by 1 to 3 heteroatoms selected from —O—, —S— and —NR²¹—, wherein R²¹ is as defined above, and wherein optionally, said ring may contain one or two unsaturated bonds which do not impart aromatic character to the ring; and wherein any of the available substitutable carbon atoms in the ring may substituted, and wherein the heterocycloalkyl ring can be substituted with 1-3 R¹⁰ groups; representative heterocycloalkyl groups include 2- or 3-tetrahydrofuranyl, 2- or 3-tetrahydrothienyl, 1-, 2-, 3- or 4-piperidinyl, 2- or 3-pyrrolidinyl, 1-, 2- or 3-piperazinyl, 2- or 4-dioxanyl, morpholinyl,

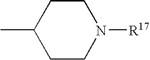

or

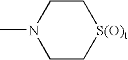

wherein R¹⁷ is as defined above and t is 0, 1 or 2.

When the optional double bond in the piperidinyl ring of formula I or IA is present, one of X¹ and X² forms the bond with the 3-position carbon and the remaining X¹ or X² is not hydrogen.

When X¹ and X² form a spiro group as defined above, the wavy lines in the structures shown in the definition indicate the points of attachment to the 4-position carbon of the piperidinyl ring, e.g., compounds of the following formulas are formed:

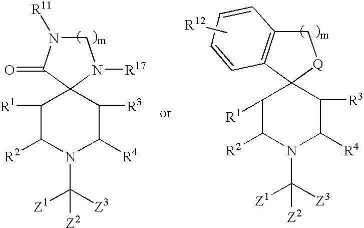

Certain compounds of the invention may exist in different stereoisomeric forms (e.g., enantiomers, diastereoisomers and atropisomers). The invention contemplates all such stereoisomers both in pure form and in mixture, including racemic mixtures.

Certain compounds will be acidic in nature, e.g. those compounds which possess a carboxyl or phenolic hydroxyl group. These compounds may form pharmaceutically acceptable salts. Examples of such salts may include sodium, potassium, calcium, aluminum, gold and silver salts. Also contemplated are salts formed with pharmaceutically acceptable amines such as ammonia, alkyl amines, hydroxyalkylamines, N-methylglucamine and the like.

Certain basic compounds also form pharmaceutically acceptable salts, e.g., acid addition salts. For example, pyrido-nitrogen atoms may form salts with strong acid, while compounds having basic substituents such as amino groups also form salts with weaker acids. Examples of suitable acids for salt formation are hydrochloric, sulfuric, phosphoric, acetic, citric, oxalic, malonic, salicylic, malic, fumaric, succinic, ascorbic, maleic, methanesulfonic and other mineral and carboxylic acids well known to those skilled in the art. The salts are prepared by contacting the free base form with a sufficient amount of the desired acid to produce a salt in the conventional manner. The free base forms may be regenerated by treating the salt with a suitable dilute aqueous base solution such as dilute aqueous NaOH, potassium carbonate, ammonia and sodium bicarbonate. The free base forms differ from their respective salt forms somewhat in certain physical properties, such as solubility in polar solvents, but the acid and base salts are otherwise equivalent to their respective free base forms for purposes of the invention.

All such acid and base salts are intended to be pharmaceutically acceptable salts within the scope of the invention and all acid and base salts are considered equivalent to the free forms of the corresponding compounds for purposes of the invention.

Compounds of formulas I and IA can be prepared by known methods from starting materials either known in the art or prepared by methods known in the art. Examples of general procedures and specific preparative examples are given below.

Typically, X¹, X²-substituted piperidines are alkylated with Z¹, Z², Z³-substituted halomethanes in the presence of excess bases such as K₂CO₃ and Et₃N, in solvents such as DMF, THF or CH₃CN, at room temperature or at elevated temperatures.

X¹, X²-substituted piperidines are either commercially available or made by known procedures. For example, 4-hydroxy-4-phenyl-piperidine can be converted to a 4-tBoc-amino-4-phenylpiperidine according to the following reaction scheme, wherein Bn is benzyl, Ph is phenyl and tBoc is t-butoxycarbonyl:

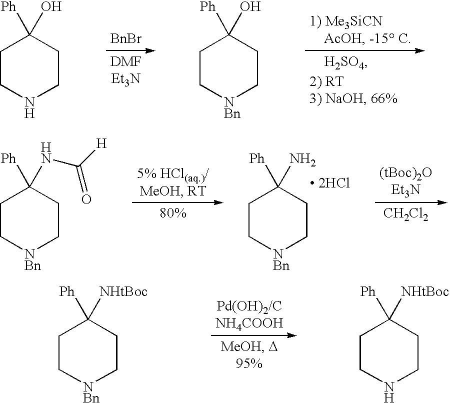

Commercially available 4-phenyl-4-piperidinol is protected with a benzyl group and the resulting intermediate is then treated with Me₃SiCN. The resultant amide is hydrolyzed with aqueous HCl in CH₃OH to produce the 4-amino compound. The amino group is protected with tBoc and the N-benzyl group is removed by hydrogenolysis to produce the desired 4-amino-piperidine derivative.

The 4-(protected)amino-piperidine then can be reacted with a Z¹, Z², Z³-halomethane and the protecting group removed. The amine (i.e., X² is —NH₂) can undergo various standard conversions to obtain amine derivatives. For example, the amine of formula I or IA can be reacted with a R²²-carboxaldehyde in the presence of a mild reducing agent such as Na(OAc)₃BH or with a compound of the formula R²²-L, wherein L is a leaving group such as Cl or Br, in the presence of a base such as Et₃N.

An alternative method for preparing compounds of formula I or IA wherein X¹ is R⁷-aryl and X² is OH involves alkylating a 4-piperidone hydrochloride with a Z¹, Z², Z³-halomethane, then reacting the ketone with an appropriately substituted R⁷-phenylmagnesium bromide or with a compound of the formula X¹-L¹, wherein L¹ is Br or I, and n-butyl-lithium.

X¹, X²-substituted compounds of formula I or IA can be converted into other compounds of formula I or IA by performing reactions well known in the art on the X¹ and/or X² substituents. For example, a carboxaldehyde-substituted piperidine (i.e., X² is —CHO) can be converted to a substituted piperidine wherein X² is R¹³—O—CH₂—, as shown in the following procedure for a compound of formula I wherein X¹ is phenyl, Z¹ and Z² are each phenyl, and R¹, R², R³ and R⁴, and Z³ are H:

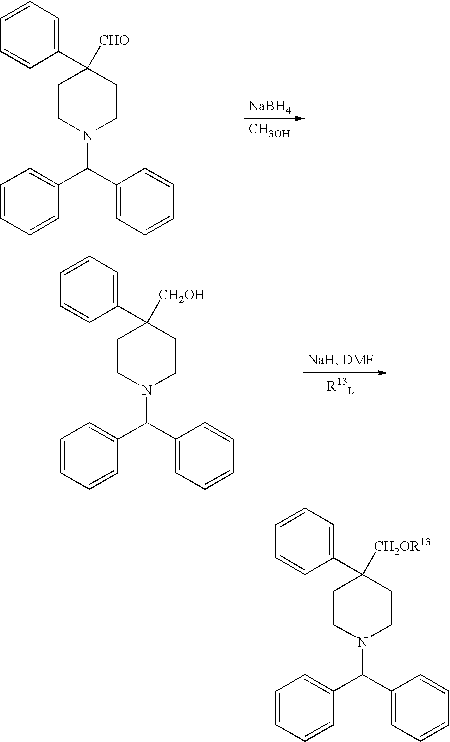

A cyano-substituted piperidine (i.e., X² is —CN) can be converted to a substituted piperidine wherein X² is R²¹R²²N—CH₂— or X² is R²⁸C(O)NH—CH₂—, as shown in the following procedure for a compound of formula I or IA wherein X¹ is phenyl, R²¹, R¹, R², R³ and R⁴, and Z³ are H, and L is a leaving group such as Cl or Br:

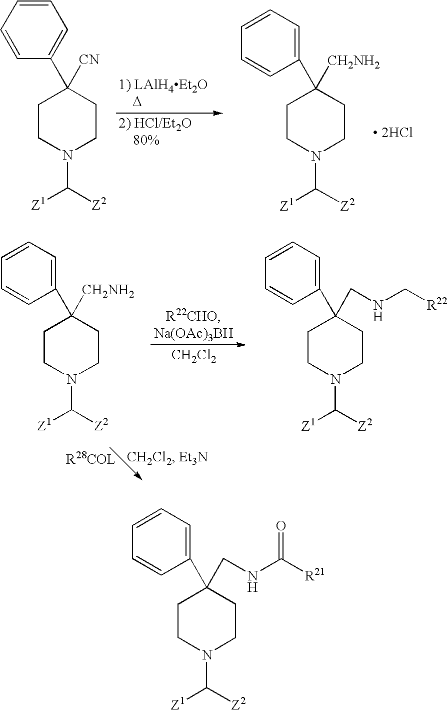

Compounds of formula I or IA wherein X¹ is a benzofused nitrogen-containing heterocycle having an R¹¹ substituent other than hydrogen are prepared by reacting the corresponding compounds wherein R¹¹ is hydrogen with a compound of the formula R¹¹L (R¹¹ is not H, and L is as defined above).

Alternatively, X¹, X²-substituted piperidine starting materials can be converted into other X¹, X²-substituted piperidines by similar procedures before reacting with the Z¹, Z², Z³-substituted halomethane.

For compounds of formula I or IA wherein R¹, R², R³ and R⁴ variously form alkylene bridges, commercially available N-protected 4-piperidones are treated with phenyl lithium and resulting intermediate is deprotected to produce the desired compounds, for example:

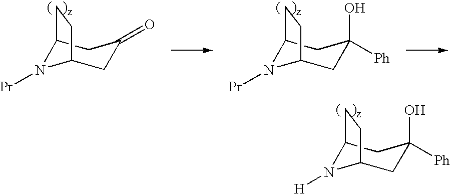

wherein Pr is a N-protecting group, Ph is phenyl and z is 1-2.

The Z¹, Z², Z³-halomethyl derivatives wherein Z¹ and Z² are R⁷-phenyl are either commercially available or can be prepared using the procedure shown in the following reaction scheme:

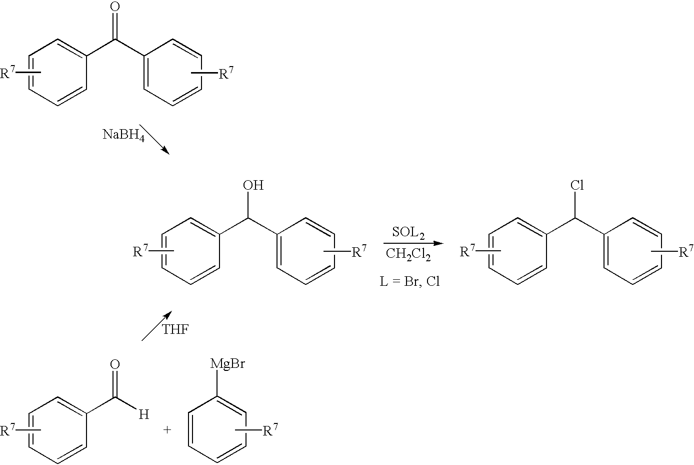

Similar procedures, or others known in the art, can be used to prepare compounds wherein the Z substituents are other than phenyl.

Compounds of the present invention and preparative starting materials thereof, are exemplified by the following examples, which should not be construed as limiting the scope of the disclosure.

The following solvents and reagents are referred to herein by the abbreviations indicated: tetrahydrofuran (THF); ethanol (EtOH); methanol (MeOH); acetic acid (HOAc or AcOH); ethyl acetate (EtOAc); N,N-dimethylformamide (DMF); and diethyl ether (Et₂O). Room temperature is abbreviated as rt.

### EXAMPLE 1

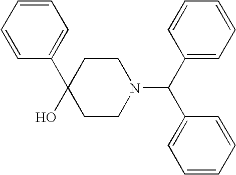

A mixture of 4-hydroxy-4-phenyl piperidine (1.5 g, 8.47 mmol) and K₂CO₃ (3.0 g, 21.73 mmol) in CH₃CN was stirred at rt. To this was added α-bromo-diphenylmethane (2.5 g, 10.12 mmol) and the reaction was stirred overnight. The reaction mixture was concentrated, redissolved in CH₂Cl₂, washed with water, dried (MgSO₄) and concentrated. Chromatography (SiO₂, 9:1 hexane/EtOAc) gave the title compound (2.6 g, 90%). ¹H NMR (CDCl₃): δ1.80 (m, 2H), 2.25 (m, 2H), 2.42 (m, 2H), 2.90 (m, 2H), 4.40 (s, 1H), 7.2-7.6 (m, 15H).

### EXAMPLE 2

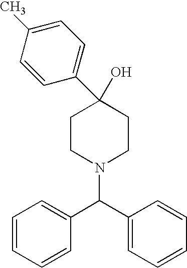

Step 1: A solution of 4-piperidone monohydrate hydrochloride (5 g, 32.6 mmol) in CH₃CN was alkylated using the procedure described in Example 1. Chromatography of the residue on silica (95:5 hexane/EtOAc) gave the desired compound.

Step 2: 4-Methylphenylmagnesium bromide (0.5 M in THF, 1.75 ml, 0.87 mmol) was added to a solution of product of Step 1 (191 mg, 0.72 mmol) in THF dropwise at 0 C. The solution was stirred at 0 for 2 h, quenched with ice-H₂O, extracted with EtOAc, washed with H₂O and brine, dried, and concentrated. Chromatography of the residue on silica (95:5 hexane/EtOAc, 93:7 hexane/EtOAc) gave the title compound (0.091 g, 30%). ¹H NMR (CDCl₃) δ7.5 (m, 6H, ArH), 7.3 (t, 4H, ArH), 7.2 (t, 4H, ArH), 4.35 (s, 1H), 2.8 (d, 2H), 2.4 (m, 5H), 2.2 (td, 2H), 1.75 (d, 2H);

MS (Cl) 358 (M+1); Elemental analysis for C₂₅H₂₇NO.1.2 H₂O: calcd: C 79.2, H 7.82, N 3.69; observed: C 78.90, H 8.02, N 3.85.

### EXAMPLE 3

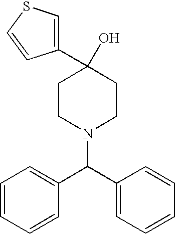

Add n-BuLi (2.5 M, 0.38 ml. 0.95 mmol) to a solution of 3-bromo-thiophene (0.15 g, 0.95 mmol) in Et₂O dropwise at −70 C. and stir for 2 h. Add a solution of the product of Step 1 of Example 2 (230 mg, 0.87 mmol) in Et₂O (4 ml) to the reaction mixture, slowly warm to rt over a period of 3 h, quench with ice-cooled NH₄Cl (aq), extract with Et₂O, wash with H₂O and brine, dry, and concentrate. Chromatograph the residue (95:5 hexane/EtOAc) to give the title compound (90 mg). ¹H NMR (CDCl₃) δ7.5 (d, 2H), 7.35 (bt, 4H), 7.25 (m, 3H), 7.2 (m, 2H), 4.4 (s, 1H), 2.8 (d, 2H), 2.5 (t, 2H), 2.3 (dt, 2H), 2.0 (d, 2H); MS (Cl) 350 (M+1); Elemental analysis for C₂₂H₂₂NOS.1.1 HCl.0.9 H₂O: calcd: C 65.11, H 6.43, N 3.54, S 7.8, Cl 9.61; observed: C 65.27, H 6.54, N 3.45, S 7.30, Cl 9.43.

### EXAMPLE 4

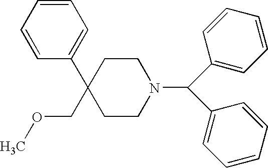

Step 1: 4-Phenyl-4-piperidinecarboxaldehyde (1.0 g, 5.29 mM) was alkylated using the procedure of Example 1, Step 1, to obtain the desired product (1.69 g, 90%). ¹H NMR (CDCl₃): δ2.40 (m, 4H), 2.50 (m, 2H), 2.85 (m, 2H), 4.25 (s, 1H), 7.20-7.50 (m, 15H), 9.42 (s,1 H).

Step 2: A solution of the product from Step 1 (3.0 g, 8.45 mmol) was cooled to 0° C. and treated with NaBH₄ (1.0 g, 26.32 mmol). After 0.5 h, reaction mixture was treated with 1N HCl and concentrated. The residue was extracted with CH₂Cl₂, dried (MgSO₄) and evaporated. Column chromatography on the residue (4:1 hexane:EtOAc) produced desired primary alcohol. ¹H NMR (CDCl₃): δ2.00 (m, 2H), 2.25 (m, 4H), 2.65 (m, 2H), 3.65 (d, 2H), 4.20 (s, 1H), 4.25 (d, 1H), 7.2-7.6 (m, 15H).

Step 3: The product of Step 2 was treated with NaH in DMF at 0° C. for 0.5 h. CH₃I was added and reaction was warmed up to rt. After stirring overnight, the reaction mixture was poured on ice, extracted with Et₂O, dried (MgSO₄) and evaporated. Column chromatography on the residue produced the title compound. ¹H NMR (CDCl₃): δ2.10 (m, 4H), 2.40 (m, 2H), 2.78 (m, 2H), 2.90 (m, 2H), 3.00(s, 3H), 4.38 (s, 1H), 7.21-7.52 (m, 15H).

### EXAMPLE 5

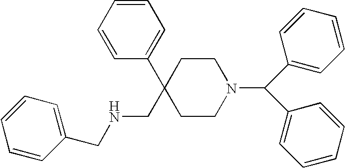

Step 1: A solution of 4-cyano-4-phenylpiperidine hydrochloride (5.0 g, 22.4 mM) in DMF (30 ml) was treated with Et₃N (7.20 ml, 47 mM) and bromodiphenylmethane (6.38 g, 25.80 mM) and stirred at rt under N₂ for 20 h. The reaction mixture was concentrated in vacuo and partitioned between EtOAc and H₂O. The organic layer was washed with twice with water, then brine, and dried (MgSO₄), filtered and concentrated. Chromatography (SiO₂, 19:1 hexane/EtOAc) gave 6.0 g (76%) of the desired product. ¹H NMR (CDCl₃): δ2.21 (m, 4H), 2.49 (t, J=12.3 Hz, 2H), 3.11 (d, J=12.5 Hz, 2H), 4.46 (s, 1H), 7.45 (m, 15H).

Step 2: A solution of the product (6.0 g, 17 mM) of Step 1 in Et₂O (40 ml) was cooled to 0° C. and treated with a 1M solution of of LAH (34.10 ml, 34 mM), dropwise, under N₂, over 0.5 h. The reaction mixture was allowed to warm to rt and then refluxed for 4 h. The reaction mixture was cooled to 0° C. and treated with water (8 eq.). The reaction mixture was allowed to warm to rt and was stirred for 1 h. The resultant solid was filtered off and rinsed with Et₂O, and the filtrate was concentrated to yield 5.45 g (90%) of desired product. ¹H NMR (CD₃OD): δ1.84 (m, 2H), 2.16 (m, 4H), 2.56 (m, 2H), 2.68 (m, 2H), 4.07 (s,1H), 7.25 (m, 15H).

Step 3: A solution of the product (0.2 g, 0.56 mM) of Step 2 in CH₂Cl₂ (3 ml) was treated with benzoyl chloride (0.078 ml, 0.673 mM) and pyridine (0.045 g, 0.568 mM) at rt for 18 h under N₂. The reaction mixture was concentrated, then partitioned between H₂O and CH₂Cl₂. The organic layer was washed with water (2×) and brine, then dried (MgSO4), filtered and concentrated. Chromatography (SiO₂, 3:1 hexane/EtOAc) gave 0.2 g (77%) of the desired product. ¹H NMR (CD₃OD): δ2.13 (m, 6H), 2.66 (m, 4H), 3.50 (s, 2H), 4.07 (s, 1H), 7.11-7.65 (m, 20H).

Step 4: A solution of the product (0.075 g, 0.16 mM) of Step 3 in THF (3 ml) was cooled to 0° C. with stirring. LAH (solid, 0.025 g, 0.65 mM) was added under N₂ and stirring was continued for 0.25 h. The reaction mixture was then refluxed for 5 h, then stirred at rt for 18 h. The reaction mixture was cooled to 0° C. and quenched with water (8 eq). The reaction mixture was allowed to warm to rt and was stirred for 1 h. The resultant solid was filtered off and rinsed with Et₂O, the filtrate was dried (MgSO₄) and concentrated. Chromatography (neutral Al₂O₃, CH₂Cl₂, then 3:1 CH₂Cl₂:EtOAc) gave 0.014 g (20%) of the title compound. ¹H NMR (CD₃OD): δ1.90 (m, 2H), 2.15 (m, 4H), 2.48 (m, 2H), 2.68 (s, 2H), 3.53 (s, 2H), 4.05 (s,1H), 7.01-7.38 (m, 20H).

### EXAMPLE 6

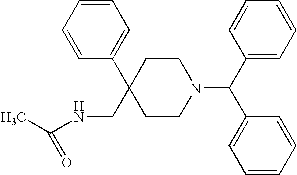

The product of Example 5, Step 2 (0.2 g, 0.561 mM), acetic anhydride (3 ml) and Et₃N (0.096 ml, 0.67 mM) were combined and stirred at rt for 18 h. The reaction mixture was concentrated and partitioned between H₂O and CH₂Cl₂. The organic layer was washed with water (2×), brine, then dried (MgSO₄), filtered and concentrated to give 0.214 g (95%) of the title compound. ¹H NMR (CD₃OD): δ1.87 (m, 5H), 2.16 (m, 4H), 2.61 (m, 2H), 3.31 (s, 2H), 4.07 (s, 1H), 7.12-7.40 (m, 20H).

### EXAMPLE 7

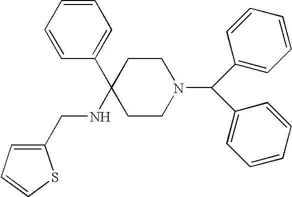

Step 1: A solution of 4-phenyl-4-hydroxy piperidine (10.0 g, 56.4 mM) in DMF (60 ml) was treated with Et₃N (8.28 ml, 59.2 mM) and benzyl bromide (7.37 ml, 62.10 mM) and stirred at rt under N₂ for 20 h. The reaction mixture was concentrated in vacuo, basified to pH 8 with saturated NaHCO₃ and partitioned between EtOAc and H₂O. The organic layer was washed twice with water, then brine, and dried (MgSO₄), filtered and concentrated. Chromatography (neutral Al₂O₃, hexane, then 1:1 hexane:EtOAc) gave 11.95 g (80%) of the desired product.

Step 2: To a mixture of the product (30.0 g, 0.112 mol) of Step 1 and (CH₃)₃SiCN (59.94 ml, 0.448 mol), cooled to −15° C. in an ethylene glycol/CO₂ bath, under N₂, is added glacial AcOH (47 ml) dropwise, while maintaining an internal temperature of −15° C. Concentrated H₂SO₄ (47 ml, 0.34 M) is added dropwise, with vigorous stirring, while maintaining an internal temperature of −15° C. The cooling bath was then removed and reaction mixture was stirred at rt for 18 h. The reaction mixture was poured on ice and adjusted to pH 7 with a 50% NaOH solution while maintaining a temperature of 25° C. The reaction mixture was then extracted with CH₂Cl₂, and the organic layer was washed with water (2×), then brine, and dried (MgSO₄), filtered and concentrated. Recrystalization with EtOAc/hexane (1:10) gave 22.35 g (68%) of desired compound. ¹H NMR (CD₃OD): δ2.10 (m, 2H), 2.40 (m, 4H), 2.82 (d, J=11.50 Hz, 2H), 3.57 (s, 2H), 7.20-7.43 (m, 10H), 8.05 (s, 1H).

Step 3: The product of Step 2 (20 g, 67.9 mM) and 5% (w/w) concentrated HCl (aq)/CH₃OH (350 ml) were stirred under N₂ for 48 h. The mixture was concentrated to yield a foam which was suspended in Et₂O and concentrated to remove excess HCl. The resultant solid was resuspended in Et₂O, collected by vacuum filtration, washed with Et₂O and dried under vacuum to give (23 g, 100%) of desired product. ¹H NMR (CD₃OD) of di-HCl salt: δ2.59 (t, J=13.3 Hz, 2H), 2.93 (t, J=13.3 Hz, 2H), 3.07 (d, J=13.50 Hz, 2H), 3.58 (d, J=13 Hz, 2H), 4.26 (s, 2H), 7.56 (m, 10H).

Step 4: The product of Step 3 (24.10 g, 71 mM), CH₂Cl₂ (300 ml), (tBoc)₂O (17.0 g, 78.1 mM) and Et₃N (14.37 g, 0.142 M) were combined and stirred under N₂, at rt, for 18 hrs. The reaction mixture was partitioned between CH₂Cl₂ and H₂O, and the aqueous layer was extracted with CH₂Cl₂. The combined organic layers were washed with water (2×), then brine, and dried (MgSO₄), filtered and concentrated. The resulting solid was suspended in Et₂O and sonicated, filtered and dried to produce the desired compound (21.98 g, 90%). ¹H NMR (CD₃OD): δ1.09 (bs, 2H), 1.39 (s, 1H), 2.05 (m, 2H), 2.34 (m, 4H), 2.65 (d, J=11.8 Hz, 2H), 3.56 (s, 2H), 7.18-7.40 (m, 10H).

Step 5: The product of Step 4 (5.22 g, 14.2 mM), CH₃OH (430 ml). Pd(OH)₂/C (3.0 g) and NH₄COOH (18.86 g, 0.298 M) were combined and refluxed under N₂ for 8 h. The reaction mixture was filtered using celite, washing with CH₃OH. The combined filtrates were concentrated to produce (3.90 g, 97%) of the desired product. 1H NMR (CD₃OD): δ1.10 (bs, 2H), 1.39 (s, 7H), 1.90 (m, 2H), 2.26 (m, 4H), 2.92 (m, 4H), 7.17-7.41 (m, 5H).

Step 6: The product of Step 5 (2.74 g, 9.91 mM), CH₃CN (85 ml), Et₃N (1.75 ml, 12.40 mM) and bromodiphenylmethane (2.70 g, 10.9 mM) were combined and stirred at rt under N₂ for 18 hrs. The mixture was concentrated and the resultant residue was partitioned between H₂O and EtOAc. The EtOAc layer was washed with water (2×), brine, then dried (MgSO₄), filtered and concentrated. Chromatography (neutral Al₂O₃, hexane, then 4:1 hexane:EtOAc) gave 2.85 g (65%) of the desired product. ¹H NMR (CD₃OD): δ1.07 (bs, 2H), 1.37 (s, 7H), 2.23 (m, 2H), 2.24 (m, 4H), 2.74 (d, J=12.1 Hz, 2H), 4.27 (s, 1H), 7.10-7.47 (m, 15H).

Step 7: The product of Step 6 (4.6 g, 10 mM), 1,4-dioxane (38 ml) and 4 M HCl in 1,4-dioxane (25 ml, 101 mM) were combined and stirred at rt under N₂ for 4 h. The mixture was concentrated and the residue was suspended in Et₂O and re-concentrated. The resultant solid was resuspended in Et₂O, sonicated and the product was collected by vacuum filtration and dried to give 3.27 g (80% of the desired product. ¹H NMR (CD₃OD) of di-HCl salt: δ2.91(m, 8H), 5.34 (s, 1H), 7.37-7.77 (m, 15H).

Step 8: To a suspension of the product of Step 7 (0.3 g, 0.722 mM) in CH₂Cl₂ (3 ml), under N₂ at rt, was added 2-thiophenecarboxaldehyde (0.133 ml, 1.44 mM). The pH of the reaction was adjusted to 6 with Et₃N and the mixture was stirred for 0.5 h. Na(OAc)₃BH (0.230 g, 1.08 mM) was then added and the reaction mixture was stirred at rt under N₂ for 3 h. The reaction was quenched with saturated NaHCO₃(aq) and partitioned between Et₂O and H₂O. The organic layer was washed with H₂O (2×), brine, dried (MgSO₄), filtered and concentrated. Chromatography (SiO₂, toluene, then 1:19 EtOAc:toluene) gave 0.158 g (50%) of the desired product. ¹H NMR (CD₃OD): δ1.96 (m, 2H), 2.17 (m, 2H), 2.52 (m, 4H), 3.45 (s, 2H), 4.24 (s, 1H), 6.76 (d. J=3.5 Hz, 1H), 6.85 (dd, J=3.6 Hz, 1H), 7.13-7.50 (m, 16H).

### EXAMPLE 8

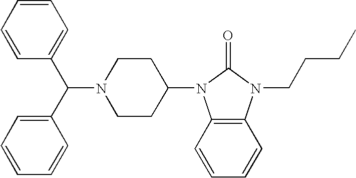

Step 1: Alkylate a solution of 4-(2-oxo-1-benzimidazolyl)-piperidine in CH₃CN using the procedure described in Step 1 of Example 1 to produce the desired compound.

Step 2: Add NaH to a solution of 3-[1-(diphenylmethyl)-4-piperidinyl]-1,3-dihydro-2H-benzimidazo-1-one (2.5 g, 6.6 mmol) in DMF (25 ml) and stir at rt for 1 h. Add n-butyl iodide to the mixture at rt and stir overnight. Quench with ice-H2O, extract with EtOAc, wash with H₂O and brine, dry (MgSO₄) and concentrate. Chromatograph the residue on silica (1:9 EtOAc/hexane) to give the title compound (2.35 g). Dissolve the title compound in Et₂O, add HCl in Et₂O (8 ml, 1 M), stir for 1 h and filter to give the HCl salt. ¹H NMR (CDCl₃) δ7.55 (m, 4H, ArH), 7.35 (m, 5H, ArH), 7.25 (m, 2H, ArH), 7.15 (m, 2H, ArH), 7.1 (m, 1H, ArH), 4.4 (m, 2H), 3.95 (t, 2H), 3.15 (d, 2H), 2.6 (dq, 2H), 2.1 (t, 2H, 1.8, m, 4H), 1.5 (m, 2H), 1.0 (t, 3H); ESI-MS 440 (M+1); Elemental analysis for C₂₉H₃₃N₃O.HCl.H₂O: calcd: C 70.5, H 7.3, N 8.5, Cl 7.18; observed: C 70.48, H 7.28, N 8.49, Cl 7.49).

### EXAMPLE 9

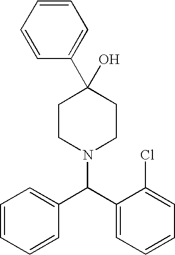

Add SOCl₂ (247 mg, 2.07 mmol) to a solution of 2-(chloro-phenyl)phenylmethanol (300 mg, 1.38 mmol) in CH₂Cl₂ at rt, stir at rt for 5 h and concentrate. Dissolve the residue in CH₃CN, add K₂CO₃, 4-hydroxy-4-phenylpiperidine and Nal. Stir the solution at reflux overnight, filter and concentrate. Chromatograph the residue on silica (9:1 hexane/EtOAc) to give the title compound. ¹H NMR (CDCl₃) δ7.91 (d, 1H), 7.58 (d, 2H), 7.54 (d, 2H), 7.42 (t, 2H), 7.32 (m, 5H), 7.26 (t, 3H), 7.16 (t, 3H), 5.0 (s, 1H), 2.8 (dd, 2H), 2.5 (dq, 2H), 2.2 (dt, 2H), 1.75 (d, 2H). Dissolve the title compound in ether, add HCl/Et₂O (1 M) to give the HCl salt. MS Cl (378 (M+1); Elemental analysis for C₂₄H₂₄NOCl.HCl.0.2H₂O:l calcd: C 68.97, H 6.13, N 3.35, Cl 16.96; observed: C 68.87, H 6.04, N 3.35, Cl 17.00.

### EXAMPLE 10

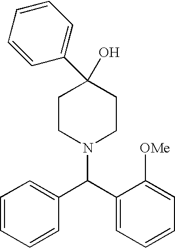

Step 1: Alkylate a solution of 4-piperidone monohydrate hydrochloride (880 mg, 5 mmol) in CH₃CN with mandelonitrile (1 g, 7.51 mmol) using the procedure described in Example 9. Chromatography of the residue on silica followed by recrystallization (EtOAc) gives the desired compound (630 mg).

Step 2: Add a solution of 2-methoxyphenylmagnesium bromide in THF (24 ml, 0.5 M, 11.85 mmol) to a solution of the product of Step 1 (330 mg, 1.185 mmol) in THF at 0 C. Remove the ice-bath and stir the reaction mixture at reflux for 6 h. Quench the reaction with NH₄Cl (aq), extract with EtOAc, wash with brine, dry and concentrate. Chromatograph the residue (95:5, 9:1 hexane/EtOAc) to give the title compound (330 mg). ¹H NMR (CDCl₃) δ7.76 (d, 1H), 7.62 (d, 1H), 7.55 (d, 1H), 7.45 (t, 1H), 7.34 (m, 3H), 7.24 (m, 2H), 7.03 (t, 1H), 6.90 (d, 2H), 4.88 (s, 1H), 3.89 (s, 3H), 2.94 (d, 1H), 2.82 (d, 1H), 2.45 (td, 2H), 2.26 (t, 2H), 1.78 (d, 2H). Dissolve the title compound in Et₂O, add HCl in Et₂O, stir for 1 h and filter to give the HCl salt. MS FAB 374.1 (M+1); elemental analysis for C₂₅H₂₇NO₂.HCl.0.15H₂O: calcd: C 72.77, H 6.91, N 3.39, Cl 8.59; obserbed: C 72.76, H 7.02, N 3.59, Cl 8.83.

### EXAMPLE 11

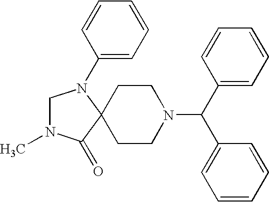

Step 1 Alkylate a solution of 1-phenyl-1,3,8-triazaspiro[4,5]decan-4-one (0.5 g) in CH₃CN using the procedure described in Step 1 of Example 1 to produce desired compound.

Step 2 Alkylate the product from Step 1, 1-phenyl-8-(diphenylmethyl)-1,3,8-triazaspiro[4,5]decan-4-one (0.4 g) with CH₃I using the procedure described in Step 2 of Example 1 to produce the title compound (0.25 g). ¹H NMR (CDCl₃) δ1.70 (d, 2H), 2.85 (m, 6H), 3.05(s, 3H), 4.50 (s, 1H), 4.72 (s, 2H), 6.95 (t, 1H), 7.05(d 2H), 7.20-7.60 (m, 12H).

Using the procedures of Examples 1 to 11, employing the appropriate starting material, compounds shown in the following tables are prepared.

TABLE 1

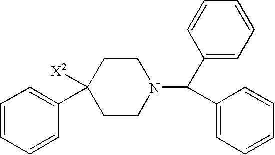

wherein X² is as defined below:

| X² | Physical Data |
|---|---|
| H | C₂₄H₂₅N |
| | FAB 283.3 (100), 167.2 52) |
| OMe | C₂₅H₂₇NO |
| | FAB 358 (80), 167 (70) |
| OEt | C₂₆N₂₉NO:HCl |
| | FAB 342 (67) 167 (100) |
| 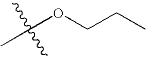 | C₂₇H₃₁NO ESI 386.1 (79), 167 (100) |
| 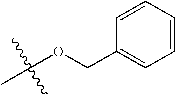 | C₃₁H₃₁NO:HCl ESI 434.2 (62), 167 (100) |
| CN | C₂₅H₂₄N₂ |
| | FAB 353.2 (53), 275.10 (24). |
| CHO | C₂₅H₂₅NO |
| | Cl 356 (28), 167 (100) |
| CH₂OH | C₂₅H₂₇NO |
| | Cl 358.1 (37), 167 (100) |
| 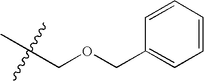 | C₃₂H₃₃NO:HCl FAB 448.1 (46), 167.2 (100) |
| CH₂OMe | C₂₅H₂₇NO |
| | FAB 357.10 (10), 167 (100) |
| CH₂OEt | C₂₆H₂₉NO |
| | Cl 373.3 (12), 372(42), 167 (100) |
| 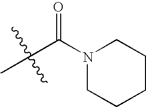 | C₃₀H₃₄NO Cl 440.25 (33), 439.2 (100), 167.2 (89) |
| CH₂NH₂ | C₂₅H₂₈N₂:2HCl |
| | ESI 357.10 (37), 167 (100) |
| CH₂NHCOCH₃ | C₂₇H₃₀N₂O |
| | ESI 399.1 (53), 167.0 (100) |
| 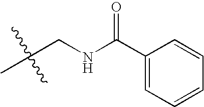 | C₃₂H₃₂N₂O FAB 462.1(15), 461.1(41), 393 (8) |
| 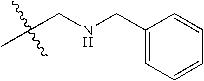 | C₃₂H₃₄N₂:HCl ESI 447.1 (100), 281.1 (29) |
| 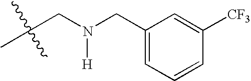 | C₃₃H₃₂N₂F₃:HCl ESI 515(100), 349.10 (33), 167 (49) |
| CH₂NHCH₂CH₃ | C₂₇H₃₂N₂:HCl |
| | ESI 385.1(100), 219.10 (26), 167 (76) |
| 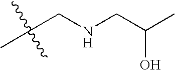 | C₂₉H₃₆N₂O:HCl Cl 429 (53), 351 (100) 327 (13), 167 (34) |
| 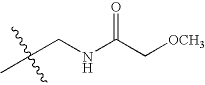 | C₂₈H₃₂N₂O₂Cl 429 (100), 351 (9), 261 (11), 167 (81) |
| 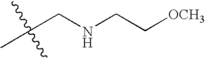 | C₂₈H₃₄N₂O:HCl Cl 415(100), 327 (33), 167 (65) |
| 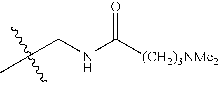 | C₃₁H₃₉N₃O:HCl ESI 470 (100), 304 (51), 259 (16), 167 (46) |
| 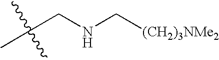 | C₃₁H₄₁N₃:HCl ESI 456 (100), 290 (11), 167 (11) |
| 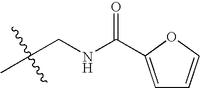 | C₃₀H₃₀N₂O₂ESI 451(100), 283 (8), 167 (94) |
| 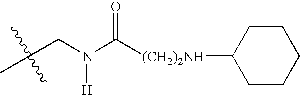 | C₃₄H₄₃N₃O:HCl ESI 510 (88), 344 (73), 167 (100) |
| 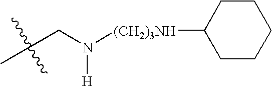 | C₃₂H₄₁N₃:HCl ESI 468 (98), 302 (22), 167 (100) |
| 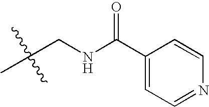 | C₃₁H₃₁N₃O:HCl Cl 462(100), 384 (4), 167 (45) |
| 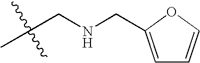 | C₃₀H₃₂N₂O:Cl ESI 437 (100), 271 (11), 167 (41) |
| 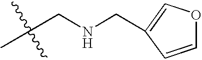 | C₃₀H₃₂N₂O:HCl ESI 437 (87), 271 (7), 167 (100) |
| 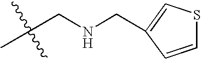 | C₃₀H₃₂N₂S:HCl ESI 453 (92), 167 (100) |
| 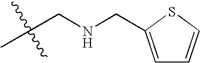 | C₃₀H₃₂N₂S:HCl ESI 453 (100), 287 (6), 167 (78) |
| 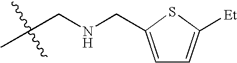 | C₃₂H₃₆N₂S:HCl ESI 481 (69), 340 (5), 167 (100) |
| 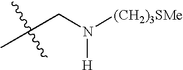 | C₂₉H₃₆N₂S:HCl ESI 445 (100), 399 (3), 279 (11), 167 (84) |
| 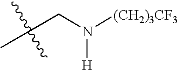 | C₂₉H₃₃N₂F₃:HCl ESI 467 (69), 167 (100) |
| CH₂NMe₂ | C₂₇H₃₂N₂:HCl |
| | FAB 385.3 (100), 219.2 (6), 162.2 (77) |
| NH₂ | C₂₄H₂₆N₂:HCl |
| | ESI 343 (48), 326 (70), 167 (100) |
| NH(CH₂)₃NEt₂ | C₃₁H₄₁N₃:HCl |
| | ESI 456 (72), 326 (74), 167 (100) |
| 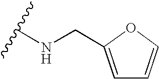 | C₂₉H₃₀N₂O:HCl Cl 423 (60), 326 (100), 167 (74) |
| 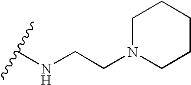 | C₃₁H₃₉N₃:HCl ESI 454(76), 326 (60), 167 (100) |
| 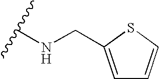 | C₂₉H₃₀N₂S:HCl FAB 439 (90), 326 (25), 167 (100) |
| NHMe | C₂₅H₂₈N₂:HCl |
| | ESI 357 (20), 326 (87), 167 (100) |
| NMe₂ | C₂₆H₃₀N₂:HCl |
| | ESI 371 (11), 326 (81), 167 (100) |

TABLE 2

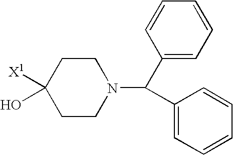

wherein X¹ is as defined below

| X¹ | Physical Data |
|---|---|
| 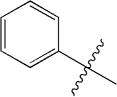 | C₂₄H₂₅NO FAB 343.1 (13), 342.1 (26) |
| 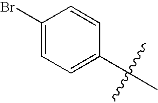 | C₂₄H₂₄BrNO ESI 424 (20) 422 (18) 167-2 (92) |
| 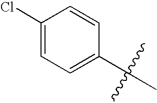 | C₂₄H₂₄NOCl Cl 363 (43), 362 (22), 167.20 (100) |
| 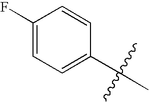 | C₂₄H₂₄FNO 361 (22), 167.2 (75) |
| Benzyl | C₂₅H₂₇NO |
| | Cl 358.1 (62), 167 (78) |
| n-Propyl- | C₂₇H₃₁NO:HCl |
| phenyl | FAB 386.1 (46), 167 (100) |
| 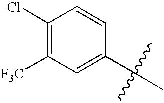 | C₂₅H₂₃NOF₃Cl El 369 (3), 368 (14), 167 (100) |
| 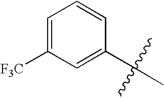 | C₂₅H₂₄F₃NO FAB 413(31), 412 (57), 167 (100) |
| 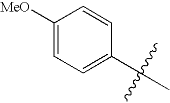 | C₂₅H₂₇NO₂Cl 374.45(M + 1), 266.30 (39%), 167.25 (100%) |
| 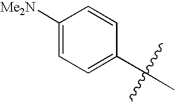 | C₂₆H₃₀N₂O FAB 387 (86%), 369 (22%) |
| 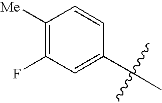 | C₂₅H₂₆NOF FAB 376.2 (68%), 375.2 (32%). 358.20 (6) |
| 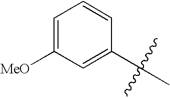 | C₂₅H₂₇NO₂Cl 374.45 (58%), 375.45 (27), 356.35 (29) |
| 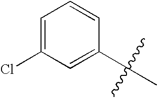 | C₂₄H₂₄ClNO Cl 378.35 (31%), 377.35 (18%), 360.30 (22) |
| 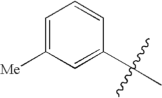 | C₂₅H₂₇NO Cl 358.35 (68), 357.35 (38), 340.35 (47), 167.25 (100) |
| 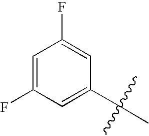 | C₂₄H₂₃F₂NO Cl 380.35(28%), 379.35 (22), 362.35 (23), 167.25 (100) |
| 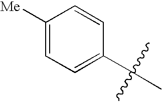 | C₂₅H₂₇NO Cl 358.35 (63), 357.35 (43), 340.35 (53), 167.25 (100) |
| 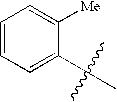 | C₂₅H₂₇NO Cl 358.35 (49), 357.35 (41), 340.35 (35), 167.25 (100) |
| 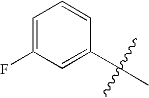 | C₂₄H₂₄FNO Cl 362.35 (41), 361.35 (218), 344.35 (39), 167.25 (100) |
| 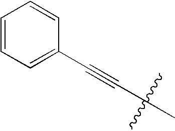 | C₂₆H₂₅NO FAB 368(37), 367 (38), 366(100), 290 (41) |
| 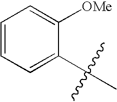 | C₂₅H₂₇NSO FAB 375 (10), 374.20 (40), 306.7 (13) |
| 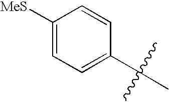 | C₂₅H₂₇NSO FAB 390 (22), 389(27), 388 (100), 312 (48) |
| 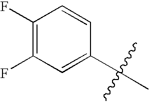 | C₂₄H₂₃NOF₂380.2 (11), 379.2 (16), 378.2 (31) |
| 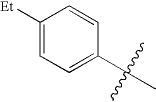 | C₂₆H₂₉NO Cl 373.45 (22), 372.40 (82), 354.35 (60), 167.25 (100) |
| 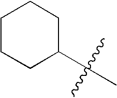 | C₂₄H₃₁NO FAB 350.3 (4), 349.3 (7), 348 917) |
| n Hexyl | C₂₄H₃₃NO |
| | FAB 352 (85), 274 (189) |
| n propyl | C₂₇H₃₁NO |
| | ESI 386 (70), 167 (100) |
| n butyl | C₂₈H₃₃NO |
| | ESI 400.1 (68), 167 (100) |
| 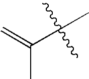 | C₂₁H₂₅NO:HCl ESI 308.1 (32), 167.0 (100) |
| 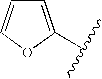 | C₂₂H₂₃NO₂:HCl Cl 334.25 (34), 333.25 (26), 316.25 (41), 167.25 (100) |
| 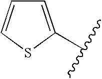 | C₂₂H₂₃NOS:HCl Cl 350.25 (32), 349.35 (24), 332.25 (41), 167.25 (100) |
| 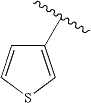 | C₂₂H₂₃NOS:HCl Cl 350.25 (27), 349.35 (18), 332.25 (20), 167.25 (100) |
| 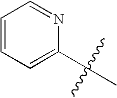 | C₂₃H₂₄N₂O:HCl ESI 345.1(68), 167 (100) |
| 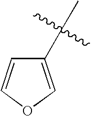 | C₂₂H₂₃NO₂Cl 334.25(37), 333.25 (24), 316.25 (31), 167.25 (100) |
| 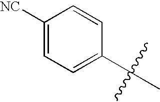 | C₂₅H₂₄N₂O:HCl FAB 369.3 (3), 368.3 (6), 367.3 (13) |
| 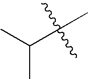 | C₂₁H₂₇NO:HCl Cl 310.40 (38), 309.40 (25), 292.40 (33), 167.25 (100) |
| 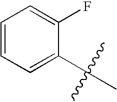 | C₂₄H₂₄NOF:HCl FAB 362.1 (100), 232.1 (11) |
| 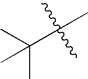 | C₂₂H₂₉NO:HCl FAB 324.30(100) |
| 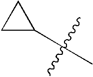 | C₂₁H₂₅NO:HCl Cl 308.2 (64), 307.2 (30), 290.2 (57), 167.25 (100) |
| 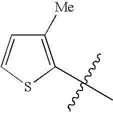 | C₂₃H₂₅NOS:HCl Cl 364.15 (69), 346.15 (71), 167.25 (100) |
| 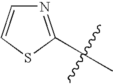 | C₂₁H₂₂N₂SO:HCl Cl 351.1 (52), 350.1 (8), 266.15 (12), 167.2 (100) |
| 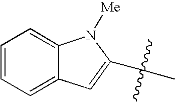 | C₂₇H₂₈N₂O:HCl FAB 397.2 (80), 167.2 (100) |
| 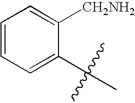 | C₂₅H₂₈N₂O:HCl ESI 373.1 (28), 167 (100) |
| 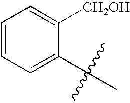 | C₂₅H₂₇NO₂:HCl ESI 374.1 (43), 167 (100) |

TABLE 3

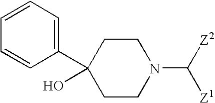

wherein Z¹ and Z² are as defined below:

| Z¹ | Z² | Physical Data |
|---|---|---|
| 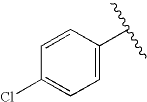 | 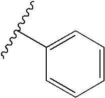 | C₂₄H₂₄NOCl Cl 380 (30), 378.1 (100), 201 (100) |
| 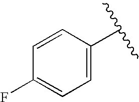 | 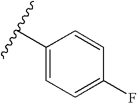 | C₂₄H₂₃NOF₂Cl 380.15 (79), 379.15 (47), 362.05 (100) |
| 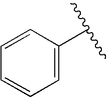 | 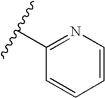 | C₂₃H₂₄N₂O:HCl ESI 345.1(69), 327.1 (49), 168 (100) |
| 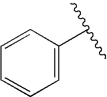 | 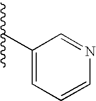 | C₂₃H₂₄N₂O:HCl ESI 345.1 (58), 168 (100) |
| 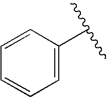 | 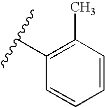 | C₂₅H₂₇NO:HCl Cl 358.20 (60), 340.20 (51), 181.25 (100) |
| 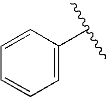 | 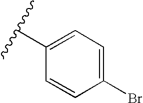 | C₂₄H₂₄NOBr:HCl ESI 424.1 (17), 422 (17), 247.1 (100), 245.1 (99) |
| 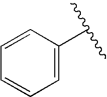 | 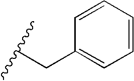 | C₂₅H₂₇NO:HCl ESI 358.1(32.70), 181 (100) |
| 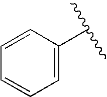 | 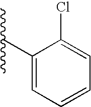 | C₂₄H₂₄NOCl:HCl Cl 380.10 (30), 378.15 (100) |
| 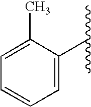 | 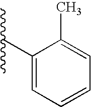 | C₂₆H₂₉NO:HCl ESI 372.1 (24), 195.1 (100) |
| 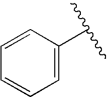 | 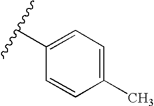 | C₂₅H₂₇NO:HCl ESI 358.1 (48%), 181.1 (100) |
| 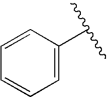 | 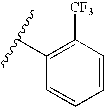 | C₂₅H₂₄ONF₃:HCl ESI 412.1 (56), 235 (100) |
| 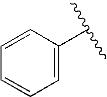 | 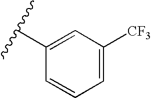 | C₂₅H₂₄ONF₃:HCl ESI 412.1 (73), 235.1 (100) |
| 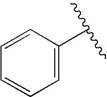 | 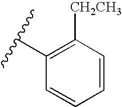 | C₂₆H₂₉NO:HCl ESI 372.1 (39), 195.1 (100) |
| 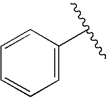 | 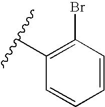 | C₂₄H₂₄NOBr:HCl ESI 424.10 (48), 422.1(47), 245.1 (100) |
| 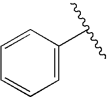 | 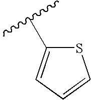 | C₂₂H₂₃NOS:HCl ESI 350.1 (31), 173 (100) |
| 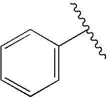 | 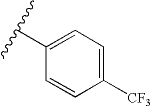 | C₂₅H₂₄ONF₃:HCl ESI 412.1 (54), 235.10 (100) |
| 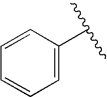 | 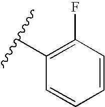 | C₂₄H₂₄NOF:HCl ESI 362.1 (23), 185.1 (100) |
| 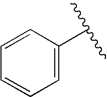 | 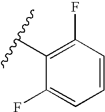 | C₂₄H₂₃NOF₂:HCl Cl 380.15 (100), 362.15 (89), 203.25 (99) |
| 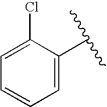 | 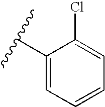 | C₂₄H₂₃NOCl₂:HCl ESI 416.1 (7), 414 (32), 412 (45), 235.1 (100) |
| 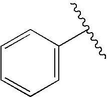 | 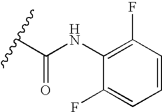 | C₂₅H₂₄N₂O₂F₂:HCl FAB 423.2 (100), 218.0 (18) |
| 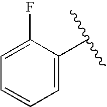 | 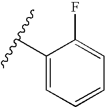 | C₂₄H₂₃NOF₂:HCl Cl 380.15 (79), 379.15 (45), 362.05 (100) |
| 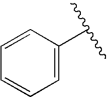 | 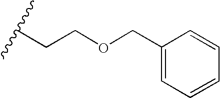 | C₂₆H₂₉NO₂:HCl FAB 388.3 (100), 266.1 (15) |
| 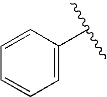 | 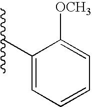 | C₂₅H₂₇NO₂:HCl FAB 374.1 (100), 197 (73) |
| 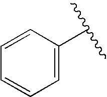 | 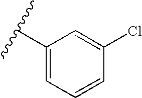 | C₂₄H₂₄NOCl:HCl FAB 380.1(27), 378.2 (80), 201.0 (100) |
| 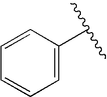 | 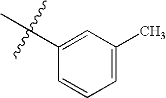 | C₂₅H₂₇NO:HCl ESI 358.1 (15), 181.1 (100) |
| Methyl | 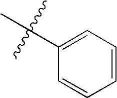 | C₁₉H₂₃NO:HCl ESI 282.1 (100), 160.0 (84.5) |
| Ethyl | 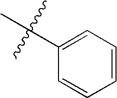 | C₂₀H₂₅NO:HCl ESI 296.1 (100), 160.0 (84) |
| 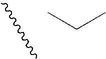 | 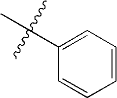 | C₂₁H₂₇NO:HCl ESI 310.1 (100), 160.1 (52) |
| 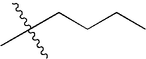 | 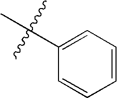 | C₂₂H₂₉NO:HCl ESI 324.1(100), 160.1 (52) |
| 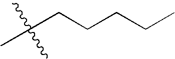 | 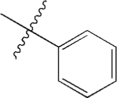 | C₂₃H₃₁NO:HCl Cl 338.3 (100), 266.20 (77), 160.35 (17) |
| 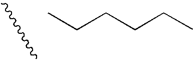 | 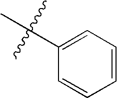 | C₂₄H₃₃NO:HCl ESI 352.1 (100), 160.0 (41.83) |
| 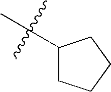 | 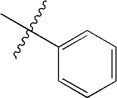 | C₂₃H₂₉NO:HCl ESI 336.1 (66.39), 160.0 (63), 159 (100) |
| 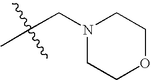 | 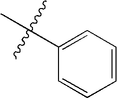 | C₂₃H₃₀N₂O₂:HCl ESI 367.1 (35), 190 (100) |
| 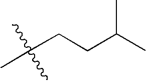 | 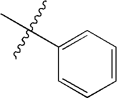 | C₂₃H₃₁NO:HCl ESI 338.1 (100), 161.0 (36), 160 (70) |

TABLE 4

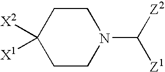

wherein X¹, X², Z¹ and Z² are as defined below

| X¹ | X² | Z¹ | Z² | Physical Data |
|---|---|---|---|---|
| 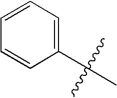 | NH₂ | 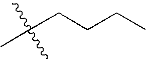 | 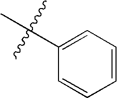 | C₂₂H₃₀N₂:HCl ESI 323(71), 306(100), 160(31) |
| 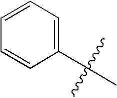 | 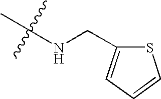 | 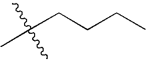 | 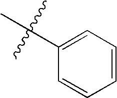 | C₂₇H₃₄N₂S:HCl ESI 419 (23), 306 (100) |
| 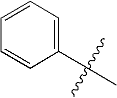 | CH₂NH₂ | 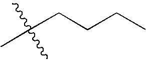 | 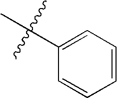 | C₂₃H₃₂N₂:HCl ESI 337 (96), 174 (100), 160 (19) |
| 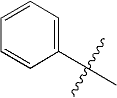 | 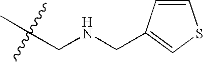 | 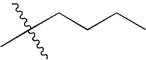 | 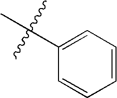 | C₂₈H₃₆N₂S:HCl ESI 433 (100), 320 (65), 174 (58) |
| 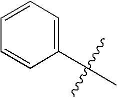 | NH₂ | 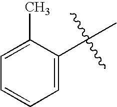 | 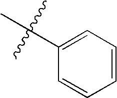 | C₂₅H₂₈N₂:HCl Cl 357 (47), 340 (24), 279 (8), 181(100) |
| 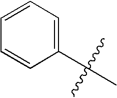 | 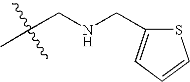 | 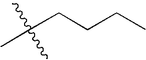 | 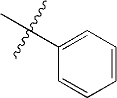 | C₂₈H₃₆N₂S:HCl ESI 433 (100), 320 (42), 174 (77) |
| 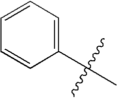 | 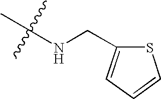 | 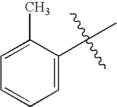 | 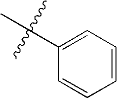 | C₃₀H₃₂N₂S:HCl ESI 453 (24), 340(27), 181 (100) |
| 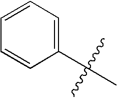 | NH₂ | 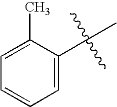 | 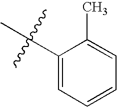 | C₂₆H₃₀N₂:HCl ESI 371 (16) 195 (100) |
| 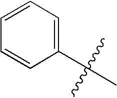 | 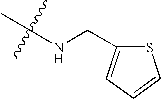 | 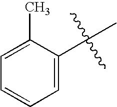 | 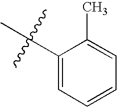 | C₃₁H₃₄N₂S:HCl ESI 467 (25), 354 (30), 195 (100) |
| 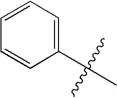 | NH₂ | 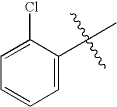 | 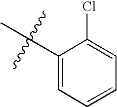 | C₂₄H₂₄N₂Cl₂:HCl ESI 413 (18), 411 (26), 396 (39), 394 (51), 237 (69), 235 (100) |
| 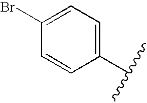 | OH | 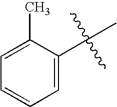 | 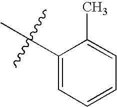 | C₂₆H₂₈BrNO:HCl 450 (12), 195.1 (100) |
| 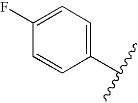 | OH | 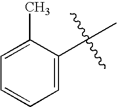 | 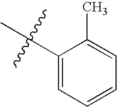 | C₂₆H₂₈FNO:HCl ESI 390.1 (9.6), 195.1 (100) |
| 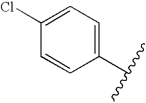 | OH | 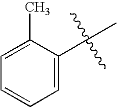 | 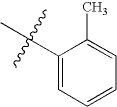 | C₂₆H₂₈ClNO:HCl 407.1 (5), 195.1 (100) 406.1 (16) |
| 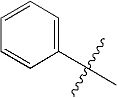 | 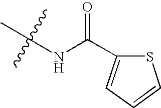 | 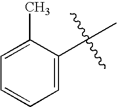 | 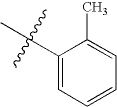 | C₃₁H₃₂N₂OS ESI 481 (25), 195 (100) |
| 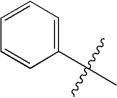 | 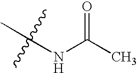 | 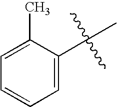 | 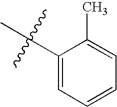 | C₂₈H₃₂N₂O Cl 413(31), 354 (8), 195 (100) |
| 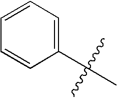 | 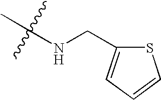 | 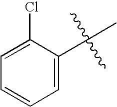 | 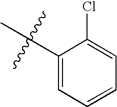 | C₂₉H₂₈Cl₂N₂S:HCl ESI 509 (10), 507 (14), 396 (56), 394 (77), 237 (68), 235 (100) |
| 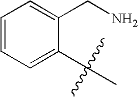 | OH | 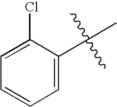 | 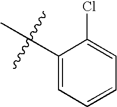 | C₂₅H₂₆N₂OCl₂:HCl ESI 443(42), 441 (56), 425 (31), 235 (100) |
| 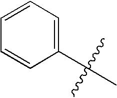 | 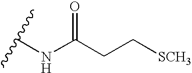 | 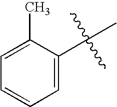 | 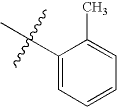 | C₃₀H₃₆N₂OS ESI 473 (39), 195 (100) |
| 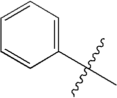 | 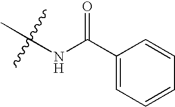 | 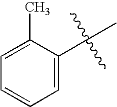 | 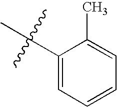 | C₃₃H₃₄N₂O ESI 475 (41), 195 (100) |
| 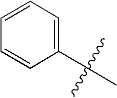 | 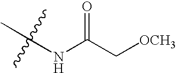 | 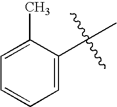 | 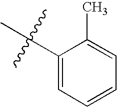 | C₂₉H₃₄N₂O₂ESI 443(31), 195 (100) |
| 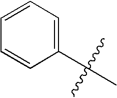 | 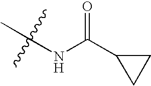 | 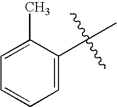 | 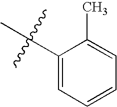 | C₃₀H₃₄N₂O:HCl ESI 439 (17), 195 (100) |
| 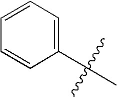 | 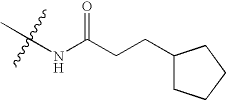 | 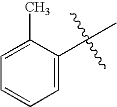 | 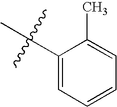 | C₃₄H₄₂N₂O:HCl ESI 495 (30), 195 (100) |
| 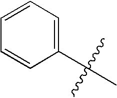 | 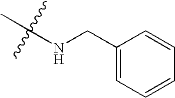 | 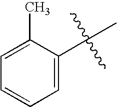 | 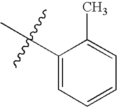 | C₃₃H₃₆N₂:HCl ESI 461 (17), 354 (28), 195 (100) |
| 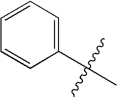 | 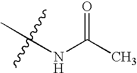 | 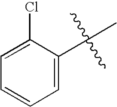 | 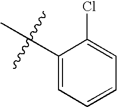 | C₂₆H₂₆N₂OCl₂ESI 455 (57), 453 (75), 396 (7), 394 (10), 237 (73), 235 (100) |
| 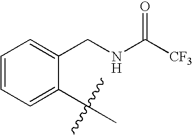 | OH | 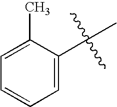 | 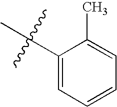 | C₂₉H₃₁N₂O₃F₃:HCl FAB 497.2 (507), 195.1 (100) |
| 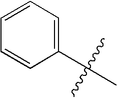 | 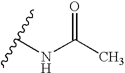 | 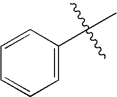 | 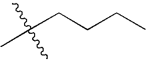 | C₂₄H₃₂N₂O:HCl ESI 365 (100), 219 (31), 160 (23) |
| 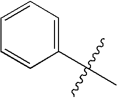 | 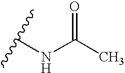 | 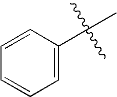 | 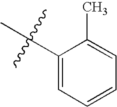 | C₂₇H₃₀N₂O:HCl ESI 399 (60), 181 (100) |
| 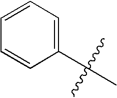 | 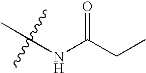 | 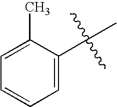 | 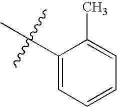 | C₂₉H₃₄N₂O:HCl ESI 427 (41), 195 (100) |
| 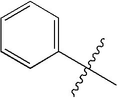 | 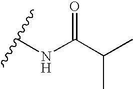 | 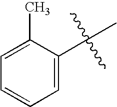 | 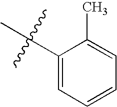 | C₃₀H₃₆N₂O:HCl ESI 441 (47), 195 (100) |
| 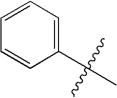 | 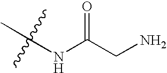 | 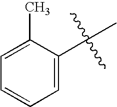 | 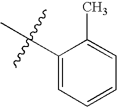 | C₂₈H₃₂N₃O:HCl ESI 428 (41), 195 (100) |
| 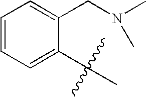 | OH | 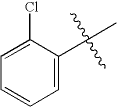 | 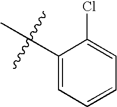 | C₂₇H₃₀Cl₂N₂O FAB 469.2 (30), 235.1 (100) |
| 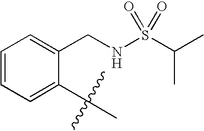 | OH | 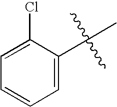 | 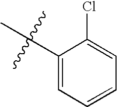 | C₂₈H₃₂Cl₂N₂O₃S Cl 549.15 (69), 548.15 (37), 547.15 (100) |
| 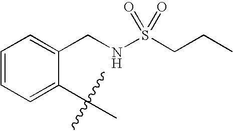 | OH | 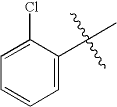 | 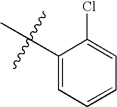 | C₂₈H₃₂Cl₂N₂O₃S FAB 549 (60), 547.1 (87) |
| 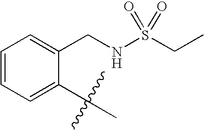 | OH | 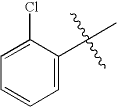 | 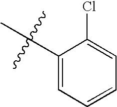 | C₂₇H₃₀Cl₂N₂O₃S FAB FAB 535 (78), 533 (100) |
| 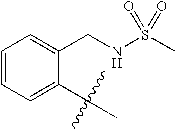 | OH | 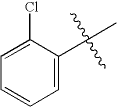 | 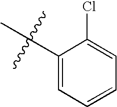 | C₂₆H₂₈Cl₂N₂O₃S FAB 523 (25) |
| 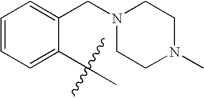 | OH | 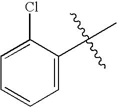 | 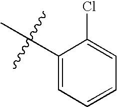 | C₃₀H₃₅Cl₂N₃O FAB 524.40(20), 330.3 (100) |
| 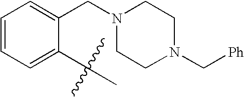 | OH | 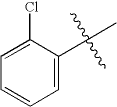 | 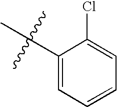 | C₃₆H₃₉Cl₂N₃O FAB 600.5 (50), 330.4 (70) |
| 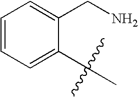 | OH | 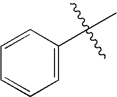 | 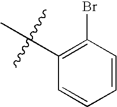 | C₂₅H₂₇BrN₂O FAB 453.2 (100), 245 (100) |
| 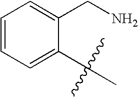 | OH | 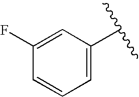 | 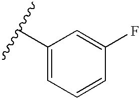 | C₂₅H₂₆N₂F₂O FAB 410.2 (25), 409.2 (100), 203.2 (50) |
| 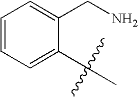 | OH | 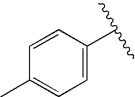 | 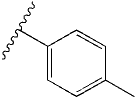 | C₂₇H₃₂N₂O FAB 401.2 (95), 195 (100) |
| 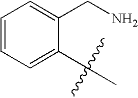 | OH | 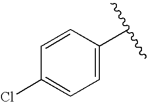 | 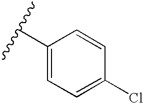 | C₂₅H₂₆Cl₂N₂O 441.1 (40), 235 (42), 157 (100) |
| 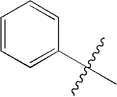 | OH | 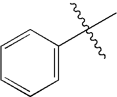 | 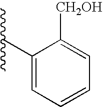 | C₂₅H₂₇NO₂Cl 374.25 (52), 356.2 (100), 178.25 (40), 160.25 (57) |
| 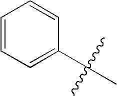 | OH | 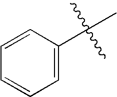 | 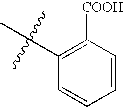 | C₂₅H₂₅NO₃FAB 388.23 (100), 210.8 (21), 168.28 (20) |
| 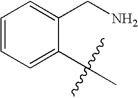 | OH | 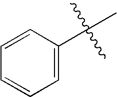 | —(CH₂)₄CH₃ | C₂₄H₃₄N₂O FAB 368.3 (30), 367.3 (100) |
| 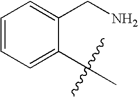 | OH | 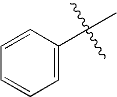 | —(CH₂)₃CH₃ | C₂₃H₃₂N₂O GAB 353.3 (100) |
| 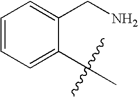 | OH | 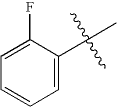 | 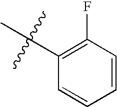 | C₂₅H₂₆N₂F₂O FAB 410.6 (35), 409.4 (98), 203.1 (65) |
| 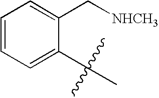 | OH | 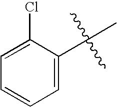 | 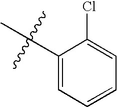 | C₂₆H₂₈Cl₂N₂O FAB 457.3 (70), 455.3 (100), 237 (30), 235.1 (52) |
| 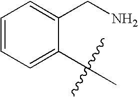 | OH | H | 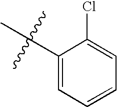 | C₁₉H₂₃N₂OCl FAB 331.2 (100), |
| 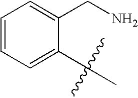 | OH | 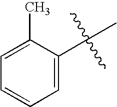 | 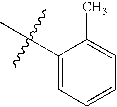 | C₂₇H₃₂N₂O FAB 402.1 (20.46), 401.1 (44.89), 195.1 (100) |
| 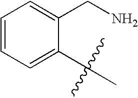 | OH | 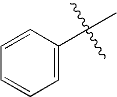 | 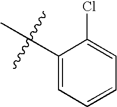 | C₂₅H₂₇ClN₂O ES 409.2 (55), 408.2 (45), 407.2 (95) |
| 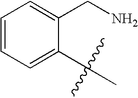 | OH | 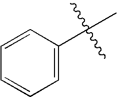 | 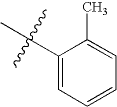 | C₂₆H₃₀N₂O ES 387 (100) |
| 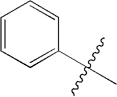 | OH | 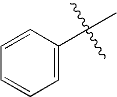 | 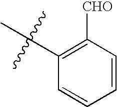 | C₂₅H₂₅NO₂Cl 372.15 (100), 354.15 (38), 195.15 (37) |
| 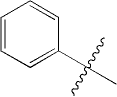 | OH | 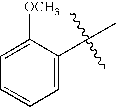 | 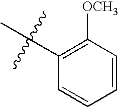 | C₂₆H₂₉NO₃FAB 404.3 (100), 227.1 (70) |
| 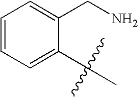 | OH | H | 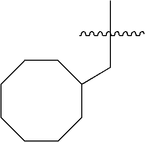 | C₂₁H₃₄N₂O FAB 331.4 (100), 266.2 (20) |
| 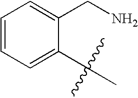 | OH | CH₃(CH₂)₃— | 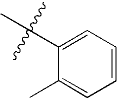 | C₂₄H₃₄N₂O FAB 367.2 (100) |
| 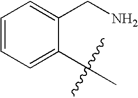 | OH | 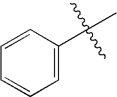 | 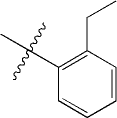 | C₂₇H₃₂N₂O ES 401.1 (46), 195.1 (100) |
| 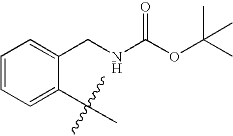 | OH | 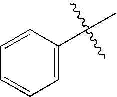 | 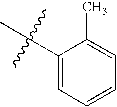 | C₃₁H₃₈N₂O₃ES 487 (100) |
| 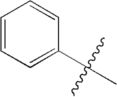 | 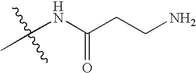 | 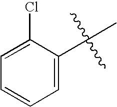 | 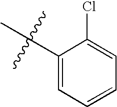 | C₂₇H₂₉Cl₂N₃O ESI 484.2 (72), 482.2 (100), 237 (60), 235.0 (65) |
| 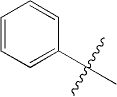 | 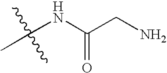 | 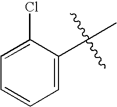 | 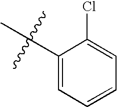 | C₂₆H₂₇Cl₂N₃O ESI 470.1 (80), 468.1 (100), 235 (78) |
| 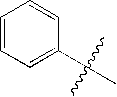 | 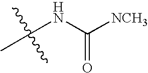 | 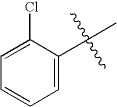 | 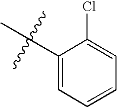 | C₂₆H₂₇Cl₂N₃O ESI 470.2 (78), 468.2 (90), 237.0 (65), 235 (100) |
| 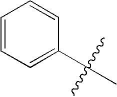 | 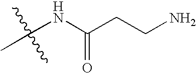 | 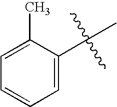 | 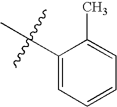 | C₂₉H₃₅N₃O ESI 442.3 (100) |
| 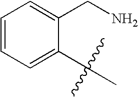 | OH | 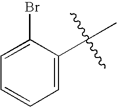 | 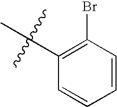 | C₂₅H₂₆N₂OBr₂ESI 533 (55), 531 (100), 324.8 (30) |

TABLE 5

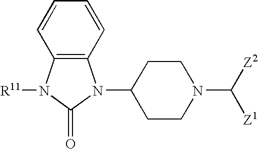

wherein R¹¹, Z¹ and Z² are as defined in the following table,
wherein Ac is acetyl, Me is methyl and Et is ethyl::

| R¹¹ | CH(Z¹)(Z²) | Physical Data |
|---|---|---|
| H | Benzhydryl | |
| 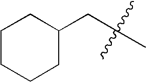 | Benzhydryl | C₃₂H₃₇N₃O:HCl Cl 480 (100), 167.25 (22) |
| 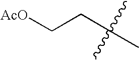 | Benzhydryl | C₂₉H₃₁N₃O₃:HCl Cl 470.15 (100), 167.25 (25) |
| 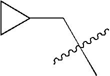 | Benzhydryl | C₂₉H₃₁N₃O:HCl Cl 438.20 (100), 167.25 (29) |
| 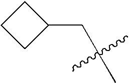 | Benzhydryl | C₃₀H₃₃N₃O:HCl FAB 452.3 (100), 167.0 (92) |
| 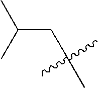 | Benzhydryl | C₂₉H₃₃N₃O:HCl Cl 440.20 (100), 167.25 (22) |
| Me | Benzhydryl | C₂₆H₂₇N₃O:HCl |
| | | Cl 398.15 (100), 167.25 (39) |
| Ethyl | Benzhydryl | C₂₇H₂₉N₃O:HCl |
| | | Cl 412.15 (100), 167.25 (32) |
| n propyl | Benzhydryl | C28H31N3O:HCl |
| | | ESI 426.1(14), 167 (100) |
| n butyl | Benzhydryl | C₂₉H₃₃N₃O:HCl |
| | | ESI 440.10 (100), 167.10 (33) |
| isopropyl | Benzhydryl | C₂₈H₃₁N₃O:HCl |
| | | ESI 446.10 (28), 167. (100) |
| 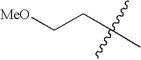 | Benzhydryl | C₂₈H₃₁N₃O₂:HCl ESI 442.10 (15), 167. (100) |
| 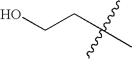 | Benzhydryl | C₂₇H₂₉N₃O₂:HCl FAB 428.3 (65), 232.1 (57) |
| H | 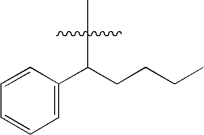 | C₂₃H₂₉N₃O:HCl ESI 364.1 (58), 218.1 (100) |
| 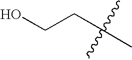 | 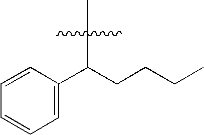 | C₂₅H₃₃N₃O₂:HCl ESI 408.1 (93), 262.1 (100) |
| n pentyl | Benzhydryl | C₃₀H₃₅N₃O:Hcl |
| | | ESI 454.1 (46), 167.1 (100) |
| n-hexyl | Benzhydryl | C₃₁H₃₇N₃O:HCl |
| | | ESI 468.1 (26), 167 (100) |
| 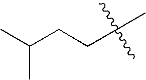 | Benzhydryl | C₂₈H₃₁N₃O₂:HCl ESI 442.10 (15), 167 (100) |
| 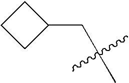 | 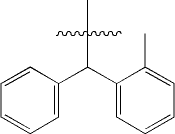 | C₃₁H₃₅N₃O:HCl ESI 466.1 (44), 181.1 (100) |
| 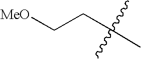 | 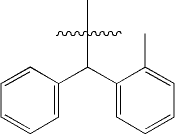 | C₂₉H₃₃N₃O₂:HCl ESI 456.1 (48), 181.10(100) |
| H | 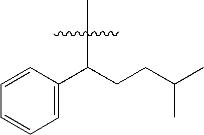 | C₂₄H₃₁N₃O:HCl Cl 378.25 (100), 306.20 (22), 218.20 (24) |
| H | 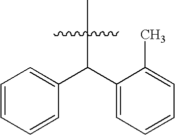 | C₂₆H₂₇N₃O:HCl ESI 398.10 (44), 181.1 (100) |
| 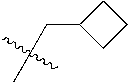 | 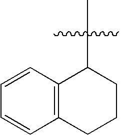 | C₂₇H₃₃N₃O:HCl ESI 416.10(36), 286.1 (39) |
| 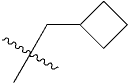 | 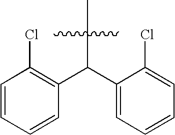 | C₃₀H₃₁N₃OCl₂:HCl ESI 522.1 (79), 521.1 (48), 520 (100) |
| 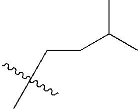 | Benzhydryl | C₃₀H₃₄N₂O:HCl Cl 439.25 (100), 168.30 (20) |
| H | 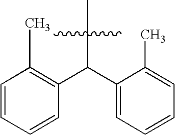 | C₂₇H₂₉N₃O:HCl Cl 412.20(32), 218.20 (42), 195.35 (100) |
| 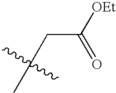 | Benzhydryl | C₂₉H₃₁N₃O₃:HCl ESI 470.1 (100), 167.1 (77.40) |
| H | 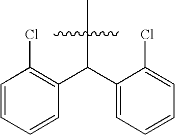 | C₂₅H₂₃N₃Cl₂O:HCl ESI 452.1 (100), 235 (85) |
| 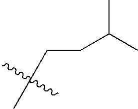 | 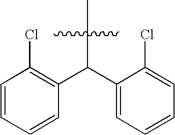 | C₃₀H₃₃N₃O₂Cl₂:HCl ESI 525.1 (39), 524.1 (82), 522 (100) |
| 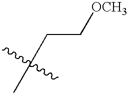 | 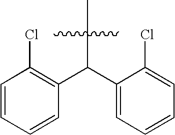 | C₂₈H₂₉N₃OCl₂:HCl ESI 511.1 (46), 510 (100), 514 (20), 513.1 (33.50) |
| 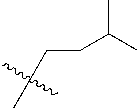 | 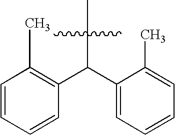 | C₃₂H₃₉N₃O:HCl ESI 482.1 (48), 195.1 (100) |
| 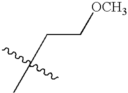 | 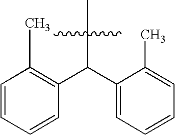 | C₃₀H₃₅N₃O₂:HCl ESI 471.1 (13), 470.1 (30), 195.1 (100) |
| H | 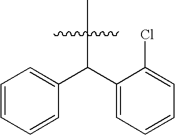 | C₂₅H₂₄N₃OCl:HCl FAB 420.2 (35), 418.2 (100), 201.0 (75) |
| H | 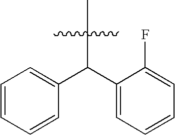 | C₂₅H₂₄N₃OF:HCl Elemental Analysis C: 68.12; H: 5.83; N: 9.48; Cl: 8.21; F;: 4.59 |
| 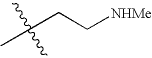 | Benzhydryl | C₂₈H₃₂N₄O:HCl ESI 442.1 (39), 441.1 (92), 167 (100) |
| 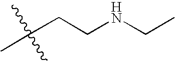 | Benzhydryl | C₂₉H₃₄N₄O:HCl ESI 455.1 (100), 290.1 (14), 289.1 (57.88), 167 (94) |
| 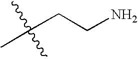 | Benzhydryl | C₂₇H₃₀N₄O:HCl ESI 428.1 (42), 427.1(97), 167 (100) |
| 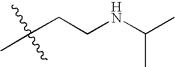 | Benzhydryl | C₃₀H₃₆N₄O.HCl ESI 470.1 (48), 469 (100), 303 (93), 167 (82.75) |
| 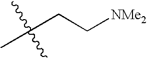 | Benzhydryl | C₂₉H₃₄N₄O:HCl ESI 457.1(13), 456 (57), 455.1 (100), 167 (72) |
| 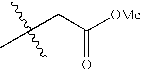 | Benzhydryl | C₂₈H₂₉N₃O₃FAB 456.2 (78), 167.0 (100) |
| 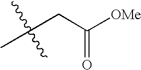 | 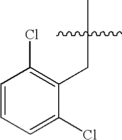 | C₂₂H₂₃Cl₂N₃O₃FAB 450.1 (27), 448.0 (100) |
| H | 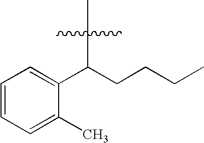 | C₂₄H₃₁N₃O FAB 378.4 (100), 218.2 (30) |
| 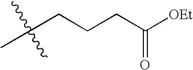 | Benzhydryl | C₃₁H₃₅N₃O₃498.2 (100), 167.1 (90) |
| 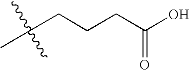 | Benzhydryl | C₂₉H₃₁N₃O₃ESI 470.1 (100), 167.1 (55) |
| 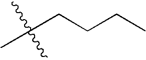 | 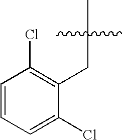 | C₂₃H₂₇Cl₂N₃O ESI 434.1 (80), 432.1 (100) |
| 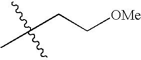 | 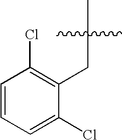 | C₂₂H₂₅Cl₂N₃O₂ESI 436.1 (58), 434.1 (100) |
| 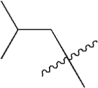 | 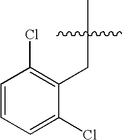 | C₂₃H₂₇Cl₂N₃O ESI 434.1 (35), 432.1 (100) |
| 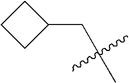 | 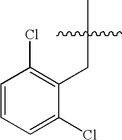 | C₂₄H₂₇Cl₂N₃O ESI 446.1 (77)), 444.1 (100) |
| 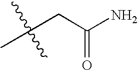 | 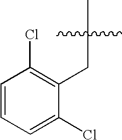 | C₂₁H₂₂Cl₂N₄O₂FAB 435.1 (78), 433.1 (100) |

TABLE 6

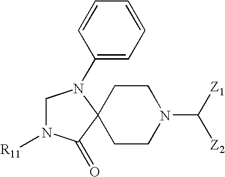

wherein R¹¹, Z¹ and Z² are as defined in the following table:

| R¹¹ | CH(Z¹)(Z²) | Physical Data |
|---|---|---|
| H | Benzhydryl | |
| 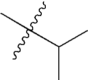 | Benzhydryl | C₂₉H₃₃N₃O ESI: 440 (100) 167 (80) |
| 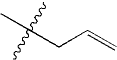 | Benzhydryl | C₂₉H₃₁N₃O ESI: 438 (100) 167 (99) |
| 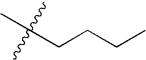 | Benzhydryl | C₃₀H₃₅N₃O ESI: 454 (100) 167 (94) |
| 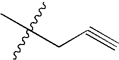 | Benzhydryl | C₂₉H₂₉N₃O ESI: 436 (99) 167 (100) |
| CH3 | Benzhydryl | C₂₇H₂₉N₃O |
| | | FAB: 412 (100) |
| 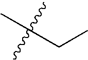 | Benzhydryl | C₂₈H₃₁N₃O FAB: 426 (100) |
| 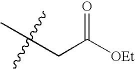 | Benzhydryl | C₃₀H₃₃N₃O₃FAB: 484 (7) 261 (14) 167 (100) |
| 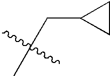 | Benzhydryl | C₃₀H₃₃N₃O ESI: 452 (100) 167 (60) |
| 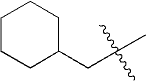 | Benzhydryl | C₃₃H₃₉N₃O ESI: 494 (100) 167 (30) |
| 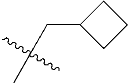 | Benzhydryl | C₃₁H₃₅N₃O.HCl FAB: 466 (100) |
| 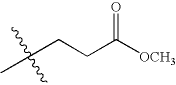 | Benzhydryl | C₃₀H₃₃N₃O₃.HCl FAB: 484 (100) 167 (41) |
| 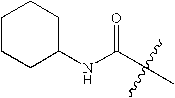 | Benzhydryl | C₃₃H₃₈N₄O₂.HCl FAB: 523 (100) |
| H | 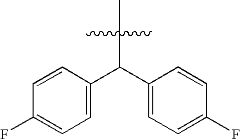 | C₂₆H₂₅N₃F₂O.HCl ESI: 434 (29) 203 (100) |
| H | 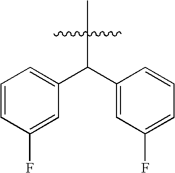 | C₂₆H₂₅N₃F₂O.HCl Cl: 434 (100) |
| H | 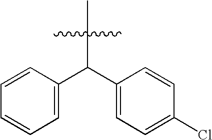 | C₂₆H₂₆N₃ClO.HCl ESI: 432 (60) 201 (100) |
| 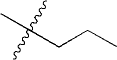 | Benzhydryl | C₂₉H₃₃N₃O.HCl ESI: 440 (100) 167 (89) |
| 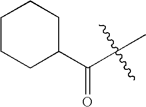 | Benzhydryl | C₃₃H₃₇N₃O₂.HCl ESI: 508 (100) 167 (35) |
| H | 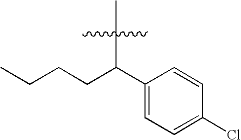 | C₂₄H₃₀N₃ClO.HCl ESI: 412 (100) 232 (92) |
| H | 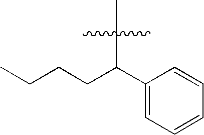 | C₂₄H₃₁N₃O.HCl ESI: 378 (100) 232 (82) |
| H | 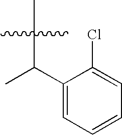 | C₂₁H₂₄N₃ClO.HCl ESI: 370 (86) 265 (100) |
| H | 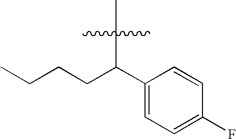 | C₂₄H₃₀N₃FO.HCl ESI: 396 (31) 232 (100) |
| H | 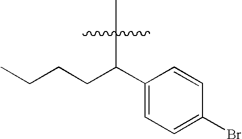 | C₂₄H₃₀N₃BrO.HCl ESI: 456 (39) 232 (100) |
| H | 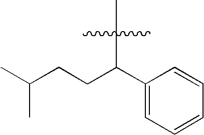 | C₂₅H₃₃N₃O.HCl ESI: 392 (73) 232 (100) |
| H | 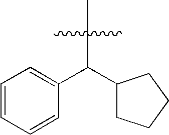 | C₂₅H₃₁N₃O.HCl FAB: 390 (100) |
| 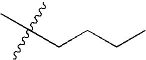 | 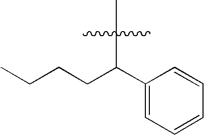 | C₂₈H₃₉N₃O.HCl ESI: 434 (68) 288 (100) |
| 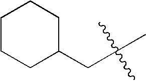 | 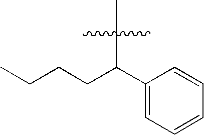 | C₃₁H₄₃N₃O.HCl ESI: 474 (90) 328 (100) |
| 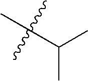 | 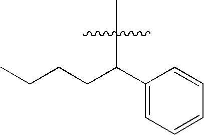 | C₂₇H₃₇N₃O.HCl ESI: 420 (81) 274 (100) |
| H | 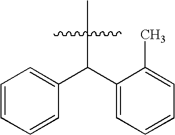 | C₂₇H₂₉N₃O.HCl FAB: 412 (25) 181 (100) |
| 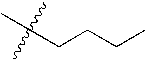 | 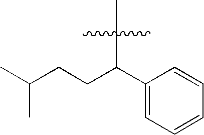 | C₂₉H₄₁N₃O.HCl ESI: 448 (97) 288 (100) |
| 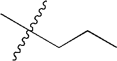 | 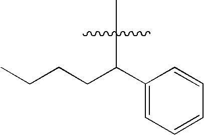 | C₂₇H₃₇N₃O.HCl ESI: 420 (62) 274 (100) |
| 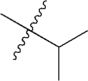 | 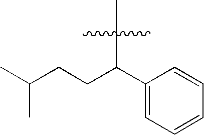 | C₂₈H₃₉N₃O.HCl ESI: 434 (66) 274 (100) |
| H | 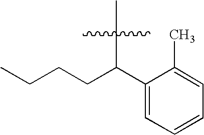 | C₂₅H₃₃N₃O.HCl ESI: 392 (59) 232 (100) |
| 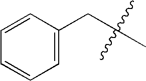 | 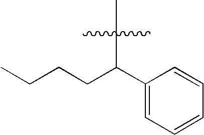 | C₃₁H₃₇N₃O.HCl ESI: 468 (100) 322 (92) |
| 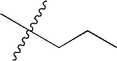 | 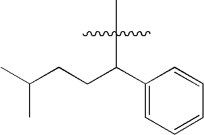 | C₂₈H₃₉N₃O.HCl ESI: 434 (100) 274 (86) |
| H | 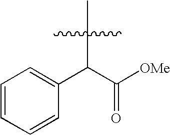 | C₂₂H₂₅N₃O₃.HCl Cl: 380 (100) |
| 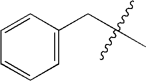 | 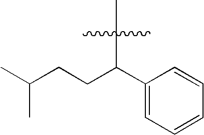 | C₃₂H₃₉N₃O.HCl ESI: 482 (100) 322 (78) |
| H | 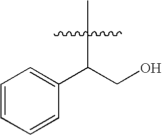 | C₂₁H₂₅N₃O₂.HCl FAB: 352 (100) |
| 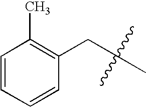 | 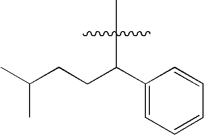 | C₃₃H₄₁N₃O.HCl FAB: 496 (100) |
| H | 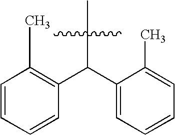 | C₂₈H₃₁N₃O.HCl ESI: 426 (19) 195 (100) |
| H | 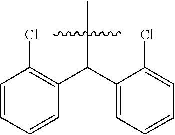 | C₂₆H₂₆N₃Cl₂O.HCl ESI: 466 (79) 235 (100) |
| H | 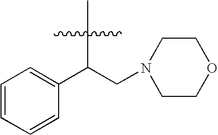 | C₂₅H₃₂N₄O₂.HCl ESI: 421 (40) 190 (100) |
| H | 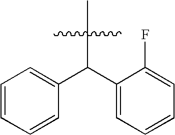 | C₂₆H₂₆N₃FO.HCl FAB: 416 (100) |
| H | 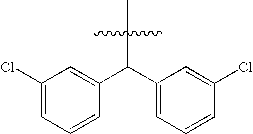 | C₂₆H₂₅N₃Cl₂O.HCl ESI: 466 (100) 235 (60) |
| H | 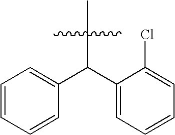 | C₂₆H₂₆N₃ClO.HCl ESI: 432 (48) 201 (100) |
| H | 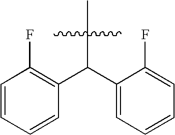 | C₂₆H₂₆N₃F₂O.HCl ESI: 434 (69) 203 (100) |
| 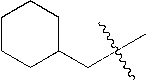 | 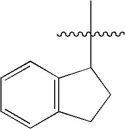 | C₂₉H₃₇N₃O.HCl ESI: 444 (52) 326 (100) |
| 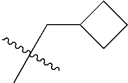 | 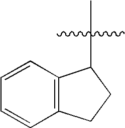 | C₂₇H₃₃N₃O.HCl ESI: 416 (33) 300 (100) |
| 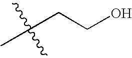 | 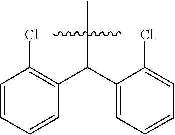 | C₂₈H₂₉N₃Cl₂O₂.HCl ESI: 510 (100) |
| 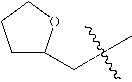 | 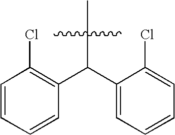 | C₃₁H₃₃N₃Cl₂O₂.HCl ESI: 550 (100) |
| 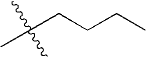 | 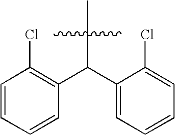 | C₃₀H₃₃N₃Cl₂O.HCl ESI: 522 (100) |
| 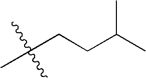 | 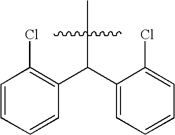 | C₃₁H₃₅N₃Cl₂O.HCl ESI: 536 (100) |
| 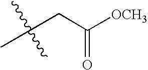 | 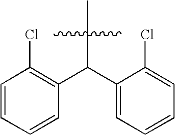 | C₂₉H₂₉N₃Cl₂O₃.HCl FAB: 538 (100) |
| 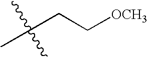 | 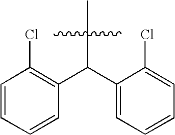 | C₂₉H₃₁N₃Cl₂O₂.HCl ESI: 524 (100) |
| 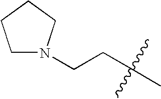 | 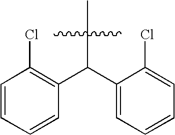 | C₃₂H₃₆N₄Cl₂O.HCl FAB: 563 (100) 235 (55) |
| 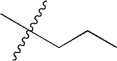 | 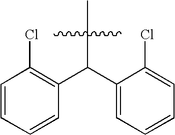 | C₂₇H₃₇N₃O₂.HCl FAB: 436 (100) |
| 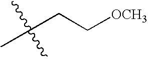 | 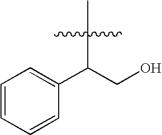 | C₂₄H₃₁N₃O₃.HCl FAB: 410 (100) |
| 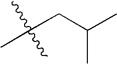 | 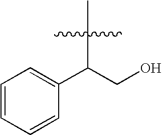 | C₂₅H₃₃N₃O₂.HCl FAB: 408 (100) |
| 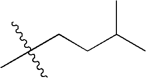 | 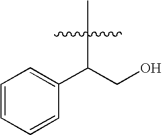 | C₂₆H₃₅N₃O₂.HCl FAB: 422 (100) |
| 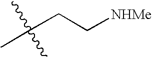 | 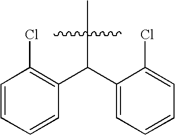 | C₂₉H₃₂N₄Cl₂O.2HCl FAB: 523 (100) |
| 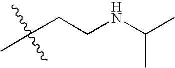 | 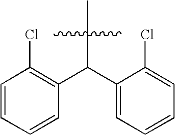 | C₃₁H₃₆N₄Cl₂O.2HCl FAB: 551 (100) |
| 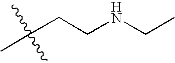 | 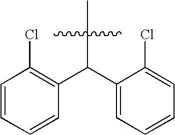 | C₃₀H₃₄N₄Cl₂O.2HCl FAB: 537 (100) |
| 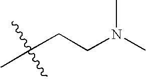 | 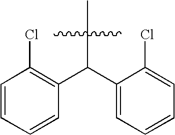 | C₃₀H₃₄N₄Cl₂O.2HCl FAB: 537 (100) |
| 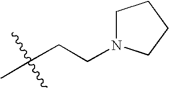 | 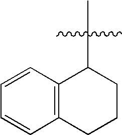 | C₂₉H₃₈N₄O.2HCl FAB: 459 (100) |
| 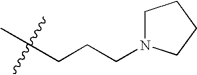 | 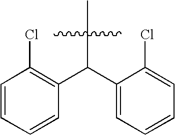 | C₃₃H₃₈N₄Cl₂O.2HCl ESI: 577 (56) 343 (100) |
| 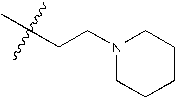 | 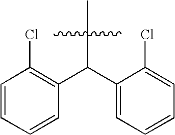 | C₃₃H₃₈Cl₂N₄O ESI 577 (100), 343 (45) |
| 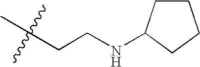 | 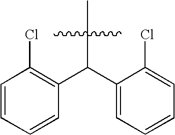 | C₃₃H₃₈Cl₂N₄O ESI 577 (100), 343 (45) |
| 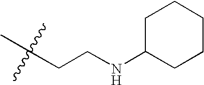 | 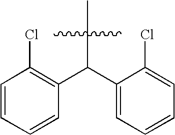 | C₃₄H₄₀Cl₂N₄O ESI 591 (100), 357 (81) |
| 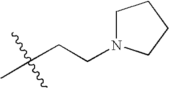 | 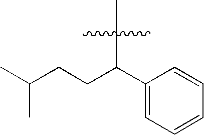 | C₃₁H₄₄N₄O ESI 487 (100), 327 (51) |
| 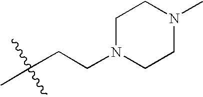 | 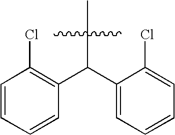 | C₃₃H₃₉Cl₂N₅O ESI 592 (100), 358 (71), 235 (64) |
| 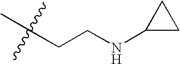 | 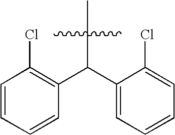 | C₃₁H₃₄Cl₂N₄O ESI 549 (100), 315 (52) |
| 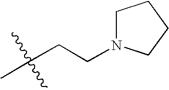 | 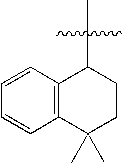 | C₃₁H₄₂N₄O ESI 487 (100), 329 (85) |
| 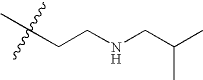 | 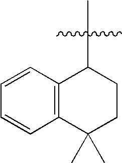 | C₃₁H₄₄N₄O ESI 489 (100), 331 (99) |
| 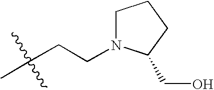 | 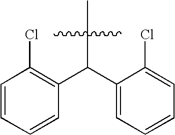 | C₃₃H₃₈Cl₂N₄O₂ESI 593 (100), 359 (45), 297 (45) |
| 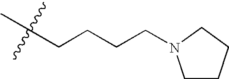 | 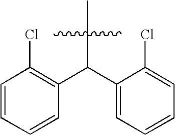 | C₃₄H₄₀Cl₂N₄O ESI 591 (100), 357 (82), 235 (99) |
| 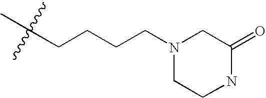 | 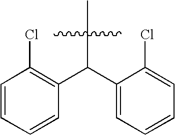 | C₃₄H₃₉Cl₂N₅O₂ESI 620 (100), 386 (12), 235 (28) |
| 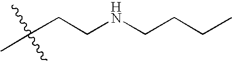 | 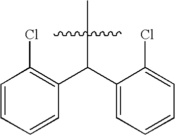 | C₃₂H₃₈Cl₂N₄O ESI 565 (100), 331 (56), 235 (52) |
| 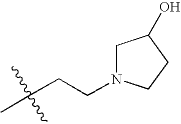 | 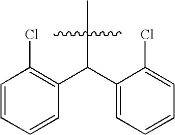 | C₃₂H₃₆Cl₂N₄O₂ESI 579 (100), 345 (51), 235 (76) |
| 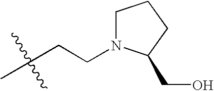 | 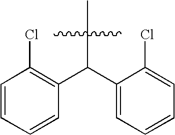 | C₃₃H₃₈Cl₂N₄O₂ESI 593 (100), 359 (63), 235 (90) |
| 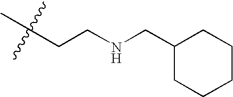 | 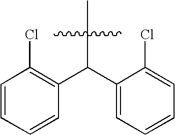 | C₃₅H₄₂Cl₂N₄O ESI 605 (100), 371 (83) |
| 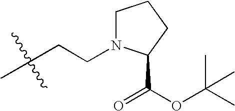 | 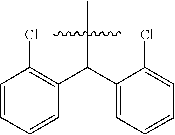 | C₃₇H₄₄Cl₂N₄O₃FAB 663 (100), 234 (42) |
| 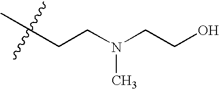 | 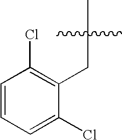 | C₂₅H₃₂Cl₂N₄O₂ESI 491 (100), 333 (29) |
| 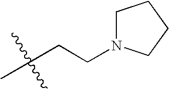 | 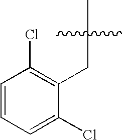 | C₂₆H₃₂Cl₂N₄O ESI 487 (100), 319 (31) |
| 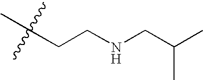 | 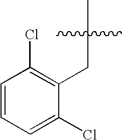 | C₂₆H₃₄Cl₂N₄O ESI 489 (100), 331 (18) |
| 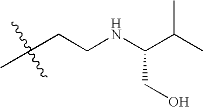 | 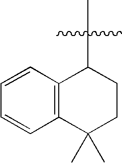 | C₃₂H₄₆N₄O₂ESI 519 (91), 361 (100) |
| 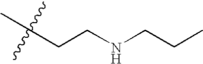 | 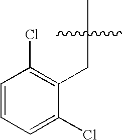 | C₂₅H₃₂N₄Cl₂O ESI 475 (100), 317 (24), 159 (69) |
| 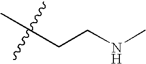 | 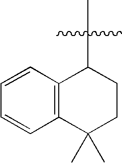 | C₂₈H₃₈N₄O FAB 447.3 (100), 289.2 (25), 242.2 (36) |
| 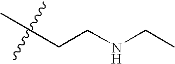 | 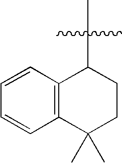 | C₂₉H₄₀N₄O FAB 461.2 (100), 303.2 (20) |
| 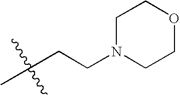 | 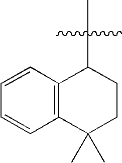 | C₃₁H₄₂N₄O₂ESI 503.1 (100), 345.1 (95) |
| 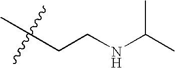 | 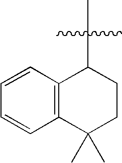 | C₃₀H₄₂N₄O ESI 475.1 (99), 317.1 (100) |
| 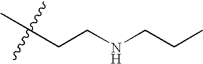 | 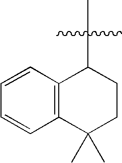 | C₃₀H₄₂N₄O ESI 4 75.1 (89), 317.1 (100) |
| 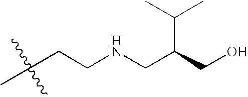 | 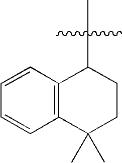 | C₃₃H₄₈N₄O₂ESI 519.1 (95), 361.1 (100)256.1 (12) |
| 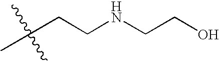 | 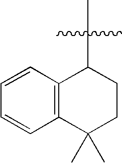 | C₂₉H₄₀N₄O₂ESI 477.1 (100), 319.1 (100) |
| 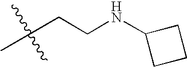 | 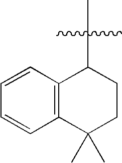 | C₃₁H₄₂N₄O ESI 487.10 (100), 329.1 (88) |
| 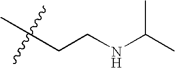 | 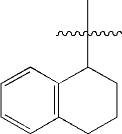 | C₂₈H₃₈N₄O FAB 447 (100), 391 (30), 317 (20) |
| 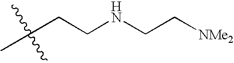 | 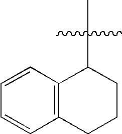 | C₂₉H₄₁N₅O FAB 476 (100), 346 (40) |
| 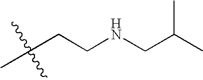 | 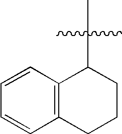 | C₂₉H₄₀N₄O FAB 461 (100), 391 (40), 167 (22) |
| 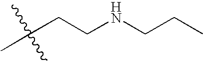 | 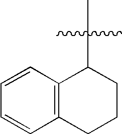 | C₂₈H₃₈N₄O FAB 447 (100), 391 (60) |
| 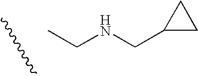 | 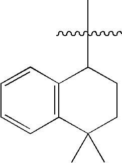 | C₃₁H₄₂N₄O ESI 487.1 (100), 329.1 (86) |
| 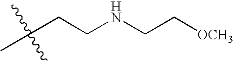 | 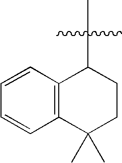 | C₃₀H₄₂N₄O₂ESI 491.1 (63), 333.10 (100) |
| 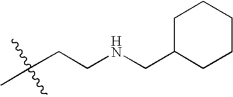 | 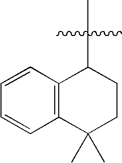 | C₃₄H₄₈N₄O ESI 529.1 (79), 371.1 (100) |
| 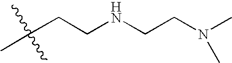 | 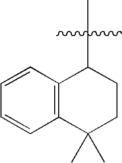 | C₃₁H₄₅N₅O ESI 504.1 (99), 358.1 (100) |
| 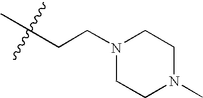 | 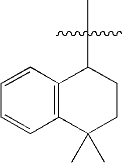 | C₃₂H₄₅N₅O ESI 516.1 (92), 358.1 (100), 251.1 (28) |
| 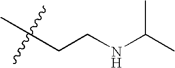 | 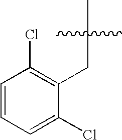 | C₂₅H₃₂Cl₂N₄O ESI 475 (100), 317 (16) |
| 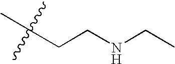 | 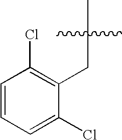 | C₂₄H₃₀Cl₂N₄O ESI 461 (100), 303 (25) |
| 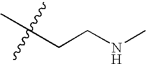 | 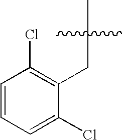 | C₂₃H₂₈Cl₂N₄O ESI 447 (100), 224 (64) |
| 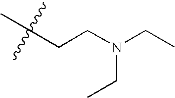 | 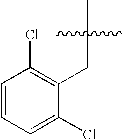 | C₂₆H₃₄Cl₂N₄O ESI 489 (100), 331 (33) |
| 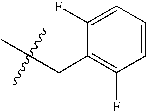 | 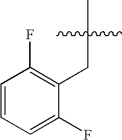 | C₂₇H₂₅F₄N₃O ESI 484 (100) |
| 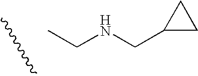 | 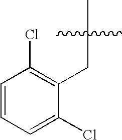 | C₂₆H₃₂Cl₂N₄O ESI 487 (100), 433 (39) |
| 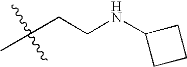 | 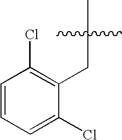 | C₂₆H₃₂Cl₂N₄O ESI 487 (100), 433 (46) |
| 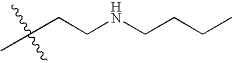 | 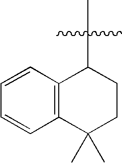 | C₃₁H₄₄N₄O ESI 489.1 (100), 331.1 (68) |
| 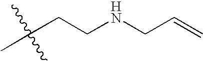 | 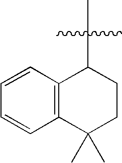 | C₃₀H₄₀N₄O ESI 473.1 (100), 315.1 (55) |
| 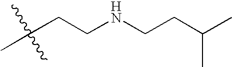 | 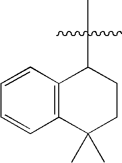 | C₃₂H₄₆N₄O ESI 503.1 (100), 345.1 (834) |
| 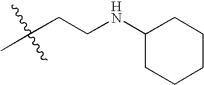 | 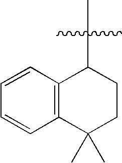 | C₃₃H₄₆N₄O ESI 515.1 (73), 357.1 (100), 258.1 (9) |
| 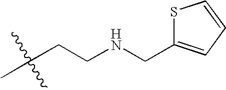 | 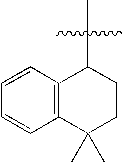 | C₃₂H₄₀N₄OS ESI 433.1 (22), 371.1 (83) |
| 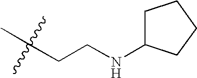 | 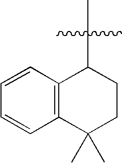 | C₃₂H₄₄N₄O ESI 501.1 (80), 343.1 (100), 251.1 (7), 159.1 (69) |
| 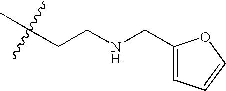 | 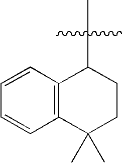 | C₃₂H₄₀N₄O₂ESI 513.1 (87), 433.1 (32), 355.1 (100), 275.1 (12) |
| 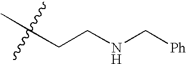 | 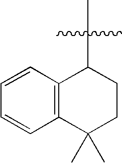 | C₃₄H₄₂N₄O ESI 523.1 (91), 365.1 (100) |
| 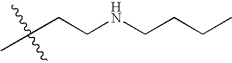 | 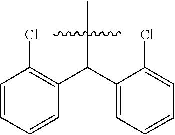 | C₃₂H₃₈Cl₂N₄O ESI 565 (100), 331 (56), 235 (52) |
| H | 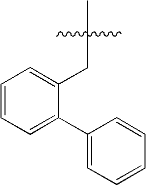 | C₂₆H₂₇N₃O ESI 398 (100), 397 (4) |
| 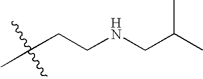 | 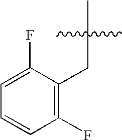 | C₂₆H₃₄FN₄O ESI 457 (92), 229 (100) |
| 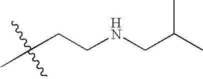 | 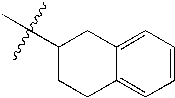 | C₂₉H₄₀N₄O ESI 461 (99), 231 (100) |
| 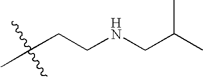 | 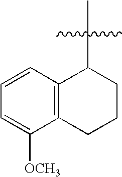 | C₃₀H₄₂N₄O₂ESI 491.1 (90), 331.1 (65), 61 (100) |
| 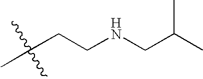 | 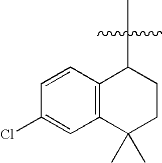 | C₃₁H₄₃ClN₄O ESI 525.1 (42), 524.1 (53), 523.1 (65), 331.1 (60), 193.1 (100) |
| 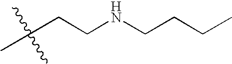 | 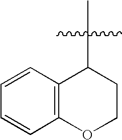 | C₂₈H₃₈N₄O₂ESI 463 (100), 331 (38) |
| 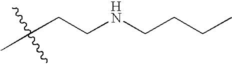 | 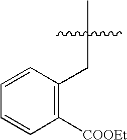 | C₂₉H₄₀N₄O₃ESI 494 (100), 247 (95) |
| 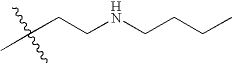 | 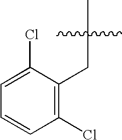 | C₂₆H₃₄Cl₂N₄O ESI 491(86) 489 (100), 245 (72) |
| 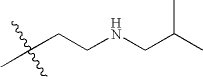 | 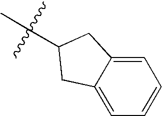 | C₂₈H₃₈N₄O ESI 447 (88), 224 (100) |
| 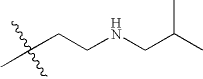 | 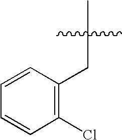 | C₂₆H₃₅ClN₄O ESI 455 (100), 228 (85) |
| 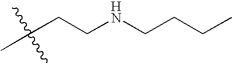 | 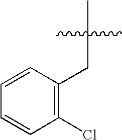 | C₂₆H₃₅ClN₄O ESI 455 (100), 228 (60) |
| 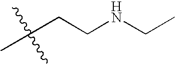 | 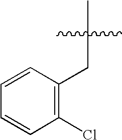 | C₂₄H₃₁ClN₄O ESI 427 (100), 303 (10), 214 (48) |
| 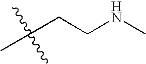 | 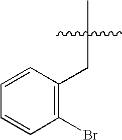 | C₂₃H₂₉BrN₄O ESI 459 (99), 457 (100), 230 (45) |
| 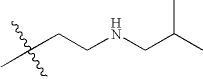 | 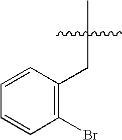 | C₂₆H₃₅BrN₄O FAB 501 (99), 499 (100), 235 (40) |
| 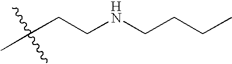 | 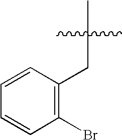 | C₂₆H₃₅BrN₄O FAB 501 (99), 499 (100), 171 (28) |
| 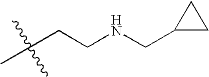 | 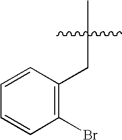 | C₂₆H₃₅BrN₄O FAB 499(99), 497 (100), 171 (20) |
| 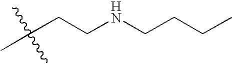 | 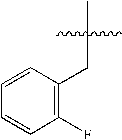 | C₂₆H₃₃FN₄O FAB 439 (100), 220 (7) |
| 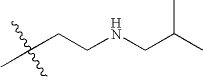 | 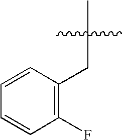 | C₂₆H₃₅FN₄O FAB 439 (100), 220 (40) |
| H | 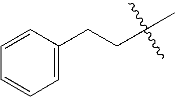 | C₂₁H₂₅N₃O FAB 336 (100), 171 (100) |
| 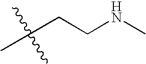 | 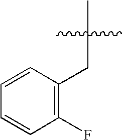 | C₂₃H₂₉FN₄O FAB 397 (100), 242 (100) |
| 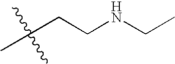 | 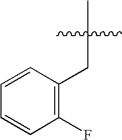 | C24H31FN4O FAB 411 (100), 242 (90) |
| H | 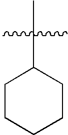 | C₁₉H₂₇N₃O FAB 314 (100), 247 (7) |
| 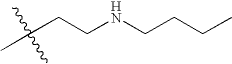 | 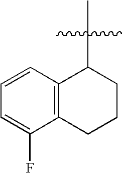 | C₂₉H₃₉FN₄O ESI 479.1(100), 424.1 (31), 331.1 (43), 203.1 (61) |
| 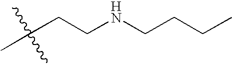 | 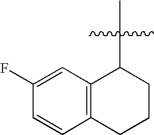 | C₂₉H₃₉FN₄O ESI 479.1(100), 424.1 (11), 331.1 (39), 203.1 (38) |
| 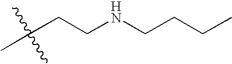 | 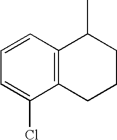 | C₂₉H₃₉ClN₄O ESI 495.1 (70), 345.1 (37), 65.0 (100) |
| H | 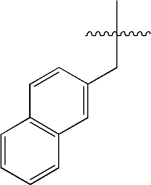 | C₂₄H₂₅N₃O ESI 372.1 (100), 200.1 (4) |
| 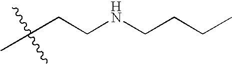 | 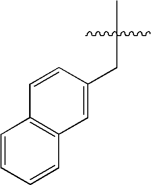 | C₃₀H₃₈N₄O ESI 471.1 (100), 331.1 (36) |
| H | 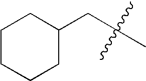 | C₂₀H₂₉N₃O ESI 328 (100) |
| H | 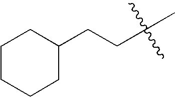 | C₂₁H₃₁N₃O ESI 342 (100) |
| H | 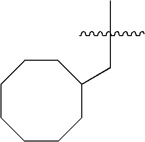 | C₂₂H₃₃N₃O ESI 356.1 (100), 171.1 (5) |
| 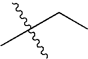 | 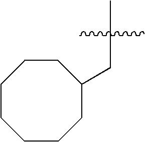 | C₂₄H₃₇N₃O ESI 370.1 (100), 247.1 (20) |

TABLE 7
compounds of the formulas shown, wherein Ph is phenyl

| Compound | Physical Data |
|---|---|
| 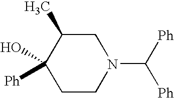 | C₂₅H₂₇NO.HCl ESI 358.1 (44.50), 167.0 (100) |
| 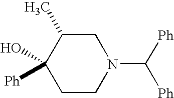 | C₂₅H₂₇NO.HCl FAB 358.2 (100), 232.1 (23.70) |
| 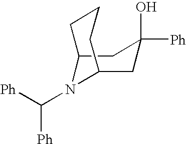 | C₂₇H₂₉NO.HCl Cl 348.20 (58), 366.25 (48) |
| 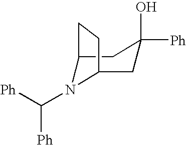 | C₂₆H₂₇NO.HCl FAB 370.1 (100), 167.0 (100) |
| 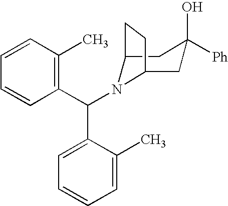 | C₂₈H₃₁NO.HCl FAB 398.1 (100), 195.1 (98) |
| 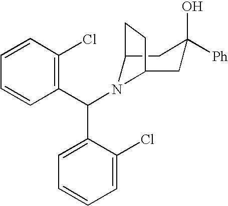 | C₂₆H₂₅NOCl₂.HCl FAB 440.1 (65), 438.0 (100), 236.9 (38), 234.9 (60) |
| 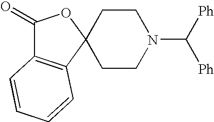 | C₂₅H₂₃NO₂.HCl FAB 370.2 (100), 292.2 (18) |
| 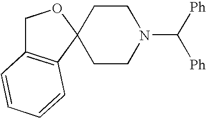 | C₂₅H₂₅NO.HCl ESI 356.1 (14.77), 168 (20.98), 167 (100) |
| 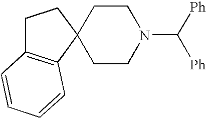 | C₂₆H₂₇N.HCl ESI 354.1 (55.06), 167.1 (100), |
| 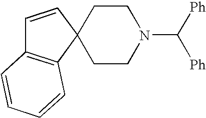 | C₂₆H₂₅N.HCl ESI 352.1 (41.94), 167.1 (100) |
| 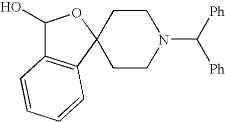 | C₂₅H₂₅NO₂.HCl ESI 372.1 (15.42), 167 (100) |
| 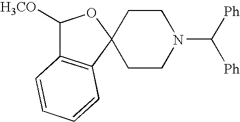 | C₂₆H₂₇NO₂.HCl Cl 386.10 (73), 354.05 (88), 167.25 (100), |
| 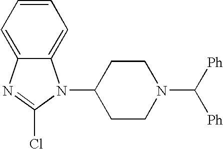 | C₂₅H₂₄N₃Cl.HCl Cl 402 (55), 366.20 (77), 250.15 (34), 167.25 (100), |
| 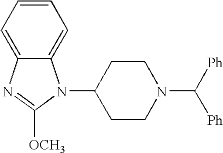 | C₂₄H₂₇N₃O.HCl Cl 398.05 (100), 232.10 (19), 167.25 (74), |
| 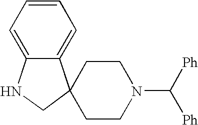 | C₂₅H₂₆N₂Cl 356.2 (26) 355.2 (100), 167(28) |
| 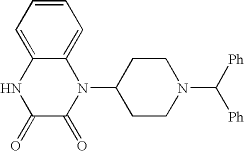 | C₂₆H₂₅N₃O₂:HCl ESI 412 (20), 167.1 (100) |
| 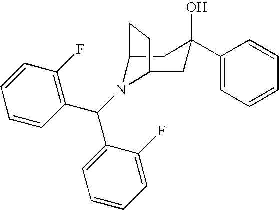 | C₂₆H₂₅F₂NO ESI 406.1 (100), 203.1 (89.11) |
| 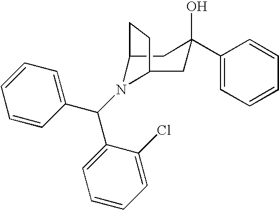 | C₂₆H₂₆ClNO ESI 406.1 (34.35), 404.10 (81.42), 201.10 (100) |
| 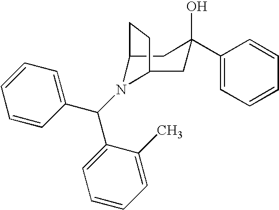 | C₂₇H₂₉NO ESI 384.1 (54.52), 181 (100) |
| 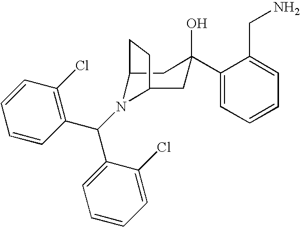 | C₂₇H₂₈Cl₂N₂O ESI 399.1 (13.87), 398.1 (56.98), 397.1 (100) |
| 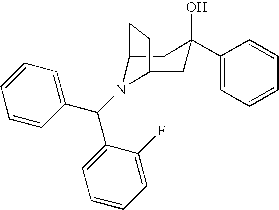 | C₂₆H₂₆FNO ESI 388.2 (90), 185.0 (100) |
| 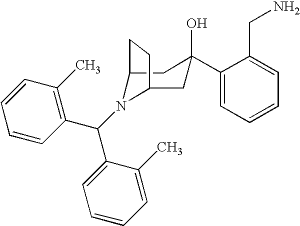 | C₂₉H₃₄N₂O ESI 429.1 (8.33), 428.10 (36.55), 427.1 (74.28) |
| 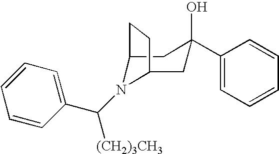 | C₂₄H₃₁NO FAB 350.4 (100), 204.3 (18) |
| 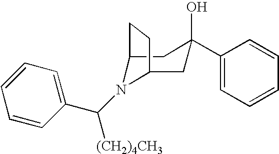 | C₂₅H₃₃NO FAB 364.40 (100), 204.3 (20) |
| 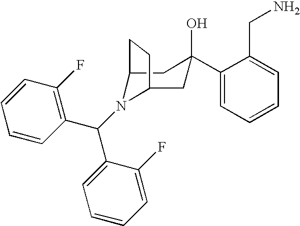 | C₂₇H₂₈F₂N₂O FAB 435.2 (100), 203.1 (55) |
| 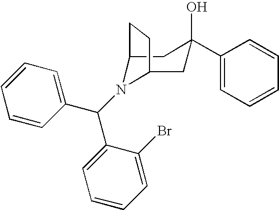 | C₂₆H₂₆BrNO FAB 448.1 (100), 247.0 (58), 166.1 (38) |
| 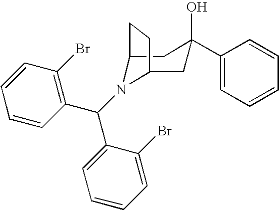 | C₂₆H₂₅Br₂NO ESI 528 (100), 325.1 (54.35) |
| 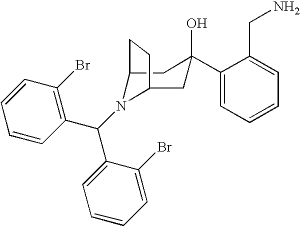 | C₂₇H₂₈Br₂N₂O FAB 560 (20), 557 (100), 324.8 (60) |
| 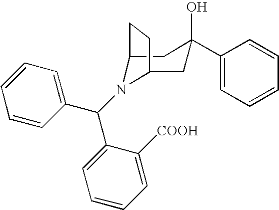 | C₂₇H₂₇NO₃Cl 414.20 (100), 396.20 (34), 211.15 (47), 186.15 (30) |
| 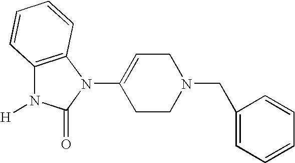 | C₁₉H₁₉N₃O ESI 306.1 (100) |
| 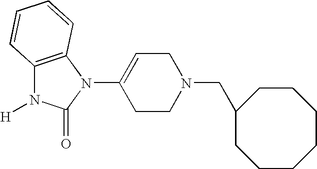 | C₂₁H₂₉N₃O ESI 341.1 (30.27), 340.1 (100) |
| 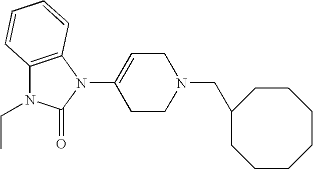 | C₂₃H₃₃N₃O ESI 369.1 (39.66), 368.1 (100) |
| 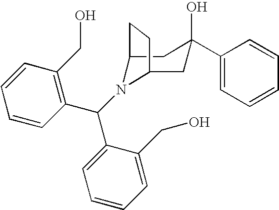 | C₂₈H₃₁NO₃ESI 430.1 (100), 204.1 (52.46) |
| 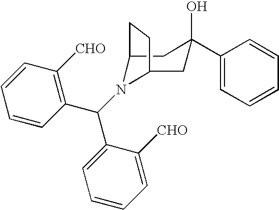 | C₂₈H₂₇NO₃FAB 426.3 (100), 225.0 (18), 195 (18) |
| 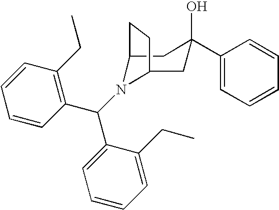 | C₃₀H₃₅NO ESI 426.1 (100), 408 (11), 223.0 (43) |
| 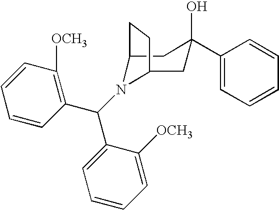 | C₂₈H₃₁NO₃ESI 430.1 (100), 412.1 (11.0), 227.0 (24.2) |
| 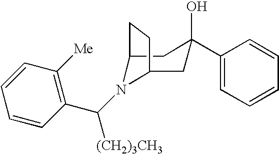 | C₂₅H₃₃NO ESI 364.10 (100), 346 (7) |
| 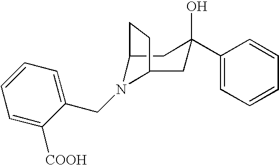 | C₂₁H₂₃NO₃FAB 338.1 (100) |
| 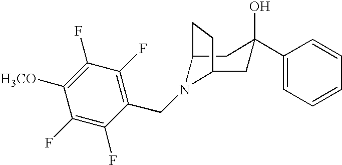 | C₂₁H₂₁F₄NO₂ESI 396.1 (100) |
| 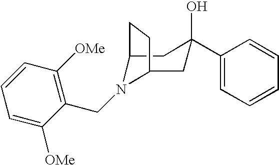 | C₂₂H₂₇NO₃CI 354 (100), 336 (78) |
| 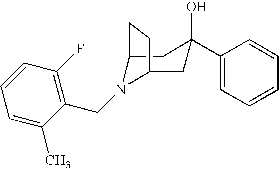 | C₂₁H₂₁F₄NO ESI 380.1 (100) |

TABLE 8

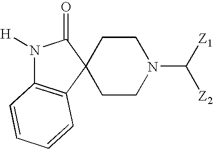

wherein Z¹ and Z² are as defined in the following table:

| Z¹ | Z² | Physical Data |
|---|---|---|
| 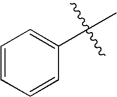 | 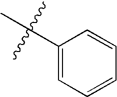 | C₂₅H₂₄N₂O.HCl FAB 369.2 (75), 167.1 (100) |
| 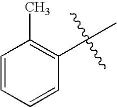 | 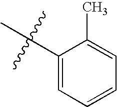 | C₂₇H₂₈N₂O.HCl FAB 397.2 (40), 195.1 (100) |
| 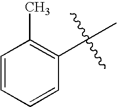 | 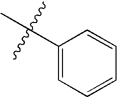 | C₂₆H₂₆N₂O.HCl ESI 383.1 (11.64), 181.1 (100) |
| 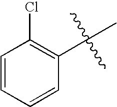 | 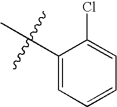 | C₂₅H₂₄N₂Cl₂O.HCl ESI 441.1 (11.05), 440.1 (15.61), 439.1 (48.02), 438.1 (23.94), 437.1 (64.05), 235.1 (100) |
| 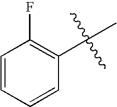 | 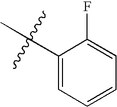 | C₂₅H₂₂N₂OF₂.HCl FAB 405.2 (100), 203.1 (76) |
| 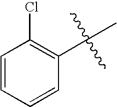 | 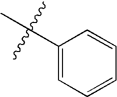 | C₂₅H₂₃ClN₂O:HCl FAB 403.1 (100) 201(70) |

Compounds of formulas II to VI can be prepared according to the procedures disclosed in the cited patent specifications.

Assays

Nociceptin Binding Assay

CHO cell membrane preparation expressing the ORL-1 receptor (2 mg) was incubated with varying concentrations of [¹²⁵I][Tyr¹⁴]nociceptin (3-500 pM) in a buffer containing 50 mM HEPES (pH7.4), 10 mM NaCl, 1 mM MgCl₂, 2.5 mM CaCl₂, 1 mg/ml bovine serum albumin and 0.025% bacitracin. In a number of studies, assays were carried out in buffer 50 mM tris-HCl (pH 7.4), 1 mg/ml bovine serum alumbin and 0.025% bacitracin. Samples were incubated for 1 h at room temperature (22° C.). Radiolabelled ligand bound to the membrane was harvested over GF/B filters presoaked in 0.1% polyethyleneimine using a Brandell cell harvester and washed five times with 5 ml cold distilled water. Nonspecific binding was determined in parallel by similar assays performed in the presence of 1 μM nociceptin. All assay points were performed in duplicates of total and non-specific binding.

Calculations of Ki were made using methods well known in the art.

For compounds of this invention, Ki values were determined to be in the range of 0.6 to 3000 nM, with compounds having a Ki value less than 10 nM being preferred. Ki values for representative compounds of the invention are as follows:

| Compounds | Ki (nM) |
|---|---|
| 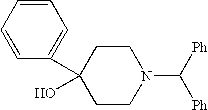 | 13 |
| 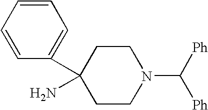 | 200 |
| 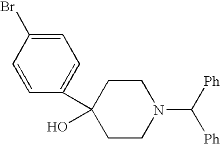 | 60 |
| 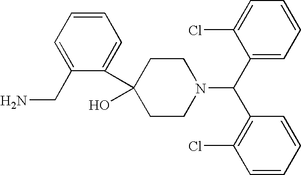 | 0.6 |
| 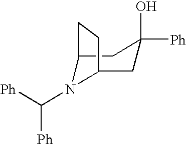 | 2.3 |
| 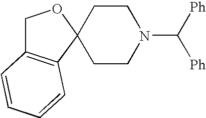 | 77 |
| 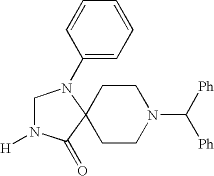 | 18 |
| 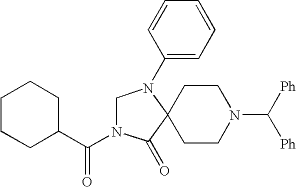 | 3,000 |

Using the procedures described in the European Journal of Pharmacology, 336 (1997), p. 233-242, the agonist activity of compounds of the invention was determined:

| | % Stimulation of |
|---|---|
| | [³⁵S]-GTPγS |
| | binding to human |
| | ORL-1 receptor |
| Compound | @ 100 nM |
| 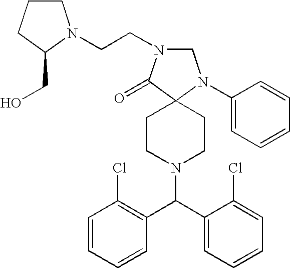 | 77 |
| 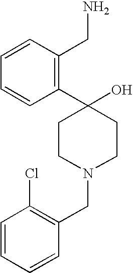 | 43 |
| 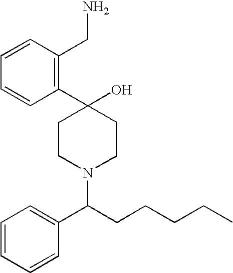 | 59 |
| 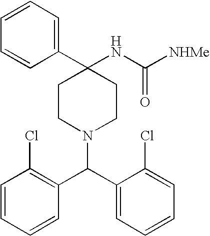 | 102 |
| 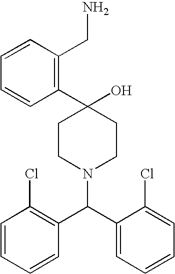 | 71 |
| 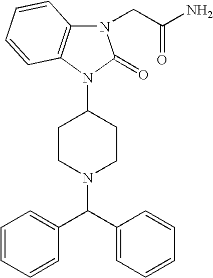 | 43 |
| 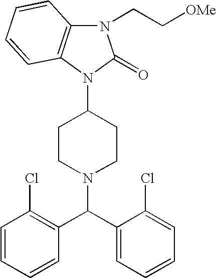 | 15 |
| 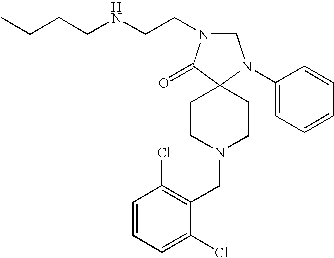 | 95 |
| 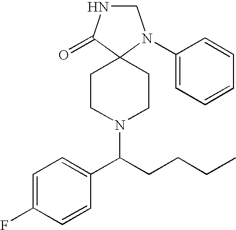 | 107 |
| 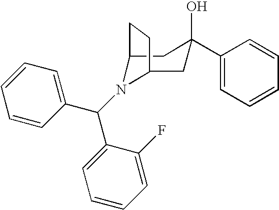 | 120 |
| 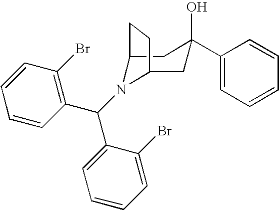 | 70 |
| 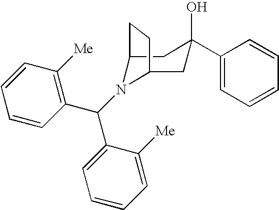 | 101 |

### EXAMPLE 12

Cough Studies

The effects of nociceptin agonist Compound A (0.3-10 mg/kg, p.o.) and Compound B (10 mg/kg, p.o.)

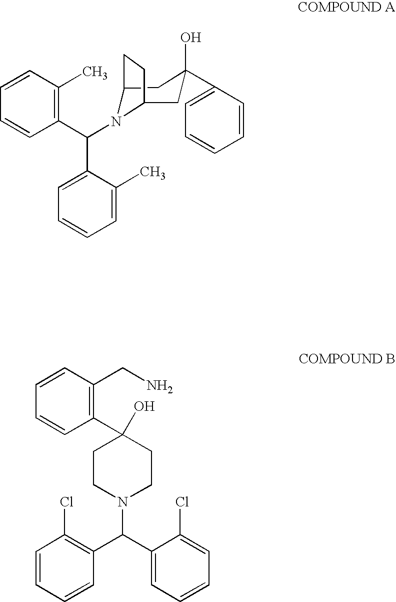

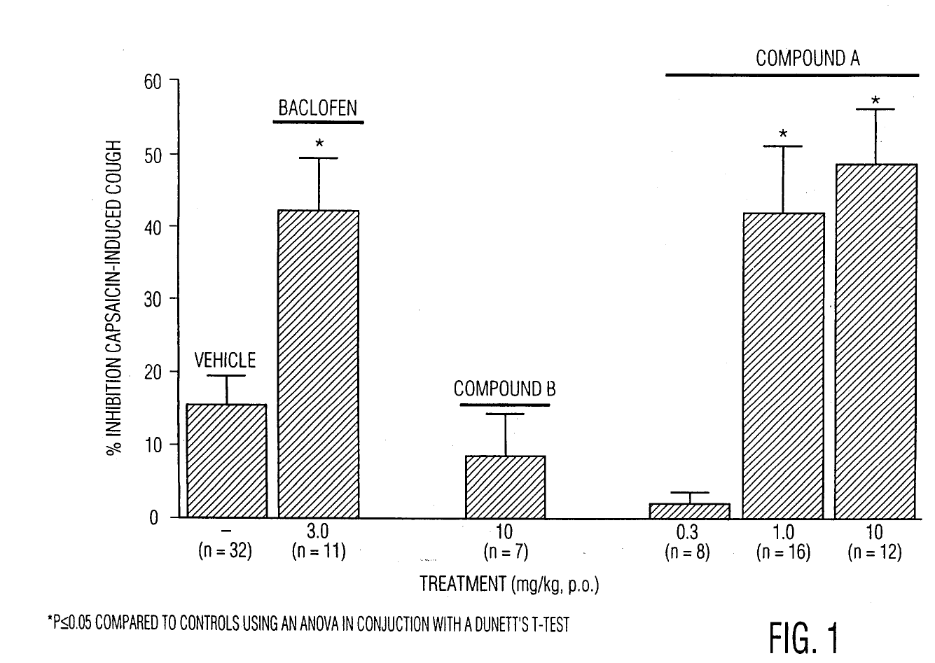

were evaluated in capsaicin-induced cough in the guinea pig according to the methods of Bolser et al. British Journal of Pharmacology (1995) 114, 735-738. This model is a widely used method to evaluate the activity of potential antitussive drugs. Overnight fasted male Hartley guinea pigs (350-450 g, Charles River, Bloomington, Mass., USA) were placed in a 12″×14″transparent chamber. The animals were exposed to aerosolized capsaicin (300 μM, for 4 min) produced by a jet nebulizer (Puritan Bennett, Lenexa, Kans., USA) to elicit the cough reflex. Each guinea pig was exposed only once to capsaicin. The number of coughs were detected by a microphone placed in the chamber and verified by a trained observer. The signal from the microphone was relayed to a polygraph which provided a record of the number of coughs. Either vehicle (methylcellulose 1 ml/kg, p.o.) or Compound A or Compound B were given 2 hours before aerosolized capsaicin. The antitussive activity of baclofen (3 mg/kg, p.o.) was also tested as a positive control. The results are summarized in the bar graph in FIG. 1.

### EXAMPLE 13

Respiratory Measurements

Studies were performed on male Hartley guinea pigs ranging in weight from 450 to 550 g. The animals were fasted overnight but given water and libitum. The guinea pigs were placed in a whole-body, head-out plethysmograph and a rubber collar was placed over the animal's head to provide an airtight seal between the guinea pig and the plethysmograph. Airflow was measured as a differential pressure across a wire mesh screen which covered a 1-in hole in the wall of the plethysmograph. The airflow signal was integrated to a signal proportional to volume using a preamplifier circuit and a pulmonary function computer (Buxco Electronics, Sharon, Conn., model XA). A head chamber was attached to the plethysmograph and air from a compressed gas source (21% O₂, balance N₂) was circulated through the head chamber for the duration of study. All respiratory measurements were made while the guinea pigs breathed this circulating air.

The volume signal from each animal was fed into a data acquisition/analysis system (Buxco Electronics, model XA) that calculated tidal volume and respiratory rate on a breath-by-breath basis. These signals were visually displayed on a monitor. Tidal volume and respiratory rate were recorded as an average value every minute.

The guinea pigs were allowed to equilibrate in the plethysmograph for 30 min. Baseline measurements were obtained at the end of this 30 min period. The guinea pigs were then removed from the plethysmograph and orally dosed with Compound A from Example 12 (10 mg/kg, p.o.), baclofen (3 mg/kg, p.o.) or a methylcellulose vehicle placebo (2 ml/kg, p.o.). Immediately after dosing, the guinea pigs were placed into the plethysmograph, the head chamber and circulating air were reconnected and respiratory variables were measured at 30, 60, 90 and 120 min post treatment. This study was performed under ACUC protocol #960103.

Data Analysis

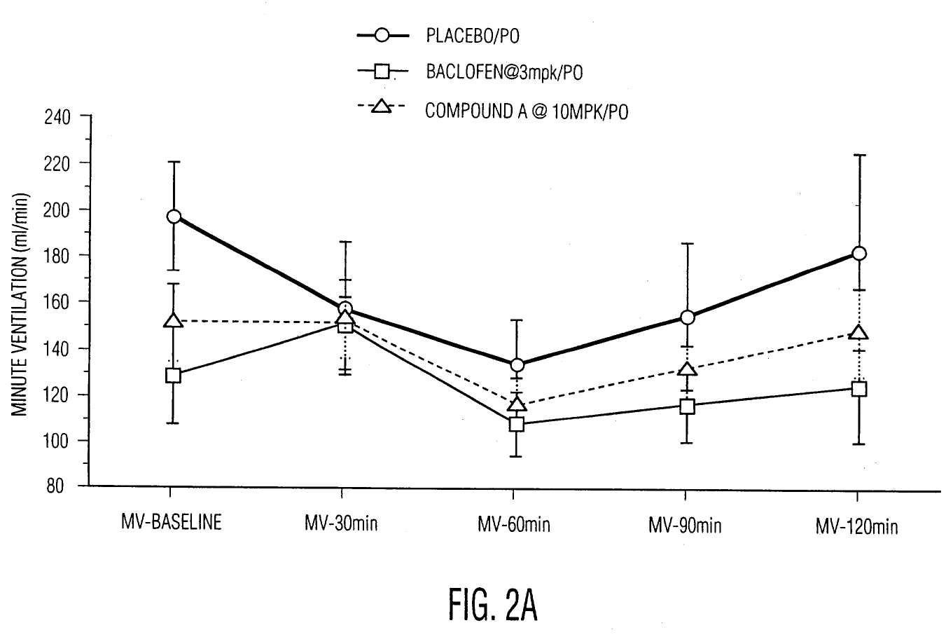

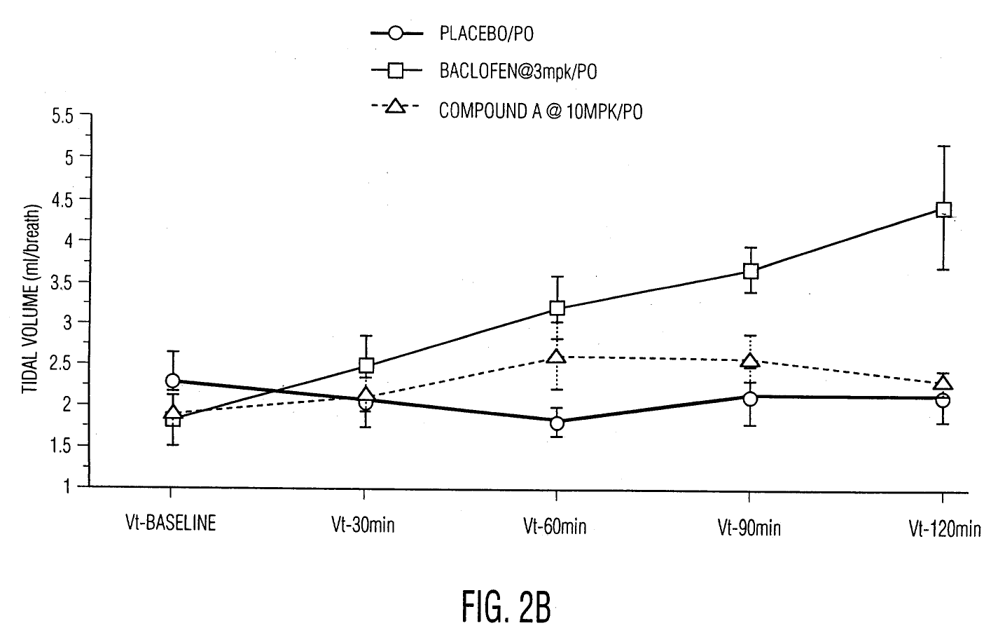

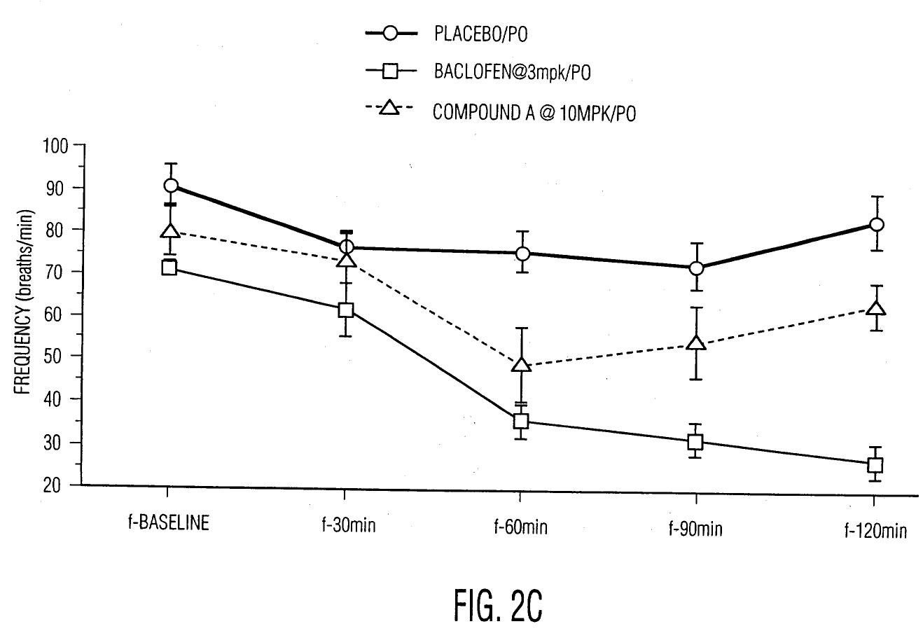

The data for tidal volume (V_{T}), respiratory rate (f) and minute volume (MV=V_{T}×f) were made for the baseline condition and at each time point after the drug or vehicle. The results are expressed as the mean ±SEM. The results are shown in FIGS. 2A, 2B and 2C. FIG. 2A shows the change in Tidal Volume, FIG. 2B shows the change in Tidal Volume and FIG. 2C shows the change in frequency of breaths.

We have surprisingly discovered that nociceptin receptor ORL-1 agonists exhibit anti-tussive activity, making them useful for suppressing coughing in mammals. The ORL-1 agonists decrease the severity and frequency of coughing. The coughing can be chronic, intractable or caused by transient medical or environmental conditions. Non-limiting examples of the causes for coughing are irritants, inflammatory diseases, influenza, asthma and upper respiratory diseases. Non-limitative examples of nociceptin receptor ORL-1 agonists include the nociceptin receptor ORL-1 agonist compounds described herein.

For mammals treated for coughing, the nociceptin receptor ORL-1 agonists may be administered along with one or more additional agents for treating cough, allergy or asthma symptoms selected from antihistamines, 5-lipoxygenase inhibitors, leukotriene inhibitors, H₃ inhibitors, β-adrenergic receptor agonists, xanthine derivatives, α-adrenergic receptor agonists, mast cell stabilizers, anti-tussives, expectorants, decongestants, NK₁, NK₂ and NK₃ tachykinin receptor antagonists, and GABA_{B} agonists. For example, the ORL-1 agonist can be administered in combination with an expectorant or an antihistamine, or with both an expectorant and an antihistamine. The combination preferably comprises 2 to 4 active agents.

Non limitative examples of antihistamines include: astemizole, azatadine, azelastine, acrivastine, brompheniramine, certirizine, chlorpheniramine, clemastine, cyclizine, carebastine, cyproheptadine, carbinoxamine, descarboethoxyloratadine (also known as SCH-34117), doxylamine, dimethindene, ebastine, epinastine, efletirizine, fexofenadine, hydroxyzine, ketotifen, loratadine, levocabastine, mizolastine, equitazine, mianserin, noberastine, meclizine, norastemizole, picumast, pyrilamine, promethazine, terfenadine, tripelennamine, temelastine, trimeprazine and triprolidine.

Non-limitative examples of histamine H₃ receptor antagonists include: thioperamide, impromidine, burimamide, clobenpropit, impentamine, mifetidine, S-sopromidine, R-sopromidine, SKF-91486, GR-175737, GT-2016, UCL-1199 and clozapine. Other compounds can readily be evaluated to determine activity at H₃ receptors by known methods, including the guinea pig brain membrane assay and the guinea pig neuronal ileum contraction assay, both of which are described in U.S. Pat. No. 5,352,707. Another useful assay utilizes rat brain membranes and is described by West et al., “Identification of Two-H₃-Histamine Receptor Subtypes,” Molecular Pharmacology, Vol. 38, pages 610-613 (1990).

The term “leukotriene inhibitor” includes any agent or compound that inhibits, restrains, retards or otherwise interacts with the action or activity of leukotrienes. Non-limitative examples of leukotriene inhibitors include montelukast [R-(E)]-1[[[1-[3-[2-(7-chloro-2-quinolinyl)-ethenyl]phenyl]-3[2-(1-hydroxy-1-methylethyl)phenyl]propyl]thio]methyl]cyclo-propaneacetic acid and its sodium salt, described in EP 0 480 717; 1-(((R)-(3-(2-(6,7-difluoro-2-quinolinyl)ethenyl)phenyl)-3-(2-(2-hydroxy-2-propyl)phenyl)thio) methylcyclopropaneacetic acid, and its sodium salt, described in WO 97/28797 and U.S. Pat. No. 5,270,324; 1-(((1(R)-3(3-(2-(2,3-dichlorothieno[3,2-b]pyridin-5-yl)-(E)-ethenyl)phenyl)-3-(2-(1-hydroxy-1-methylethyl)phenyl) propyl)thio) methyl)cyclopropaneacetic acid, and its sodium salt, described in WO 97/28797 and U.S. Pat. No. 5,472,964; pranlukast, N-[4-oxo-2-(1H-tetrazol-5-yl)-4H-1-benzopyran-8-yl]-p-4-phenylbutoxy) benzamide) described in WO 97/28797 and EP 173,516; zafirlukast, (cyclopentyl-3-[2-methoxy-4-[(o-tolylsulfonyl) carbamoyl]benzyl]-1-methylindole-5-carbamate) described in WO 97/28797 and EP 199,543; and [2-[[2(4-tert-butyl-2-thiazolyl)-5-benzofuranyl] oxymethyl]phenyl]acetic acid, described in U.S. Pat. No. 5,296,495 and Japanese patent JP08325265 A.

The term “5-lipoxygenase inhibitor” or “5-LO inhibitor” includes any agent or compound that inhibits, restrains, retards or otherwise interacts with the enzymatic action of 5-lipoxygenase. Non-limitative examples of 5-lipoxygenase inhibitors include zileuton, docebenone, piripost, ICI-D2318, and ABT 761.

Non-limitative examples of β-adrenergic receptor agonists include: albuterol, bitolterol, isoetharine, mataproterenol, perbuterol, salmeterol, terbutaline, isoproterenol, ephedrine and epinephrine.

A non-limitative example of a xanthine derivative is theophylline.

Non-limitative examples of x-adrenergic receptor agonists include arylalkylamines, (e.g., phenylpropanolamine and pseudephedrine), imidazoles (e.g., naphazoline, oxymetazoline, tetrahydrozoline, and xylometazoline), and cycloalkylamines (e.g., propylhexedrine).

A non-limitative example of a mast cell stabilizer is nedocromil sodium.

Non-limitative examples of anti-tussive agents include codeine, dextromethorphan, benzonatate, chlophedianol, and noscapine.

A non-limitative example of an expectorant is guaifenesin.

Non-limitative examples of decongestants are pseudoephedrine, phenylpropanolamine and phenylephrine. Non-limitative examples of NK₁, NK₂ and NK₃ tachykinin receptor antagonists include CP-99,994 and SR 48968.

Non-limitatve examples of GABA_{B} agonists include baclofen and 3-aminopropyl-phosphinic acid.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 70 percent active ingredient. Suitable solid carriers are known in the art, e.g. magnesium carbonate, magnesium stearate, talc, sugar, lactose. Tablets, powders, cachets and capsules can be used as solid dosage forms suitable for oral administration.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Liquid form preparations include solutions, suspensions and emulsions. As an example may be mentioned water or water-propylene glycol solutions for parenteral injection.

Liquid form preparations may also include solutions for intranasal administration.

Aerosol preparations suitable for inhalation may include solutions and solids in powder form, which may be in combination with a pharmaceutically acceptable carrier, such as an inert compressed gas.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably the compound is administered orally.

Preferably, the pharmaceutical preparation is in unit dosage form. In such form, the preparation is subdivided into unit doses containing appropriate quantities of the active component, e.g., an effective amount to achieve the desired purpose.

The quantity of active compound in a unit dose of preparation may be varied or adjusted from about 0.1 mg to 1000 mg, more preferably from about 1 mg. to 300 mg, according to the particular application.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small increments until the optimum effect under the circumstances is reached. For convenience, the total daily dosage may be divided and administered in portions during the day if desired.

The amount and frequency of administration of the compounds of the invention and the pharmaceutically acceptable salts thereof will be regulated according to the judgment of the attending clinician considering such factors as age, condition and size of the patient as well as severity of the symptoms being treated. A typical recommended dosage regimen for compounds of formula I is oral administration of from 10 mg to 2000 mg/day preferably 10 to 1000 mg/day, in two to four divided doses to provide relief from pain, anxiety, depression, asthma or alcohol abuse. The compounds are non-toxic when administered within this dosage range.

For treating cough, the amount of nociceptin receptor ORL-1 agonist in a unit dose is preferably from about 0.1 mg to 1000 mg, more preferably, from about 1 mg to 300 mg. A typical recommended dosage regimen is oral administration of from 1 mg to 2000 mg/day, preferably 1 to 1000 mg/day, in two to four divided doses. When the nociceptin receptor ORL-1 agonist and one or more additional agents are administered together, they are preferably administered in a combined dosage form (e.g., a single tablet), although they can be administered separately. The additional agents are administered in amounts effective to provide relief from cough, allergy or asthma symptoms, preferably from about 0.1 mg to 1000 mg, more preferably from about 1 mg to 300 mg per unit dose. A typical recommended dosage regimen of the additional agent is from 1 mg to 2000 mg/day, preferably 1 to 1000 mg/day, in two to four divided doses.

The following are examples of pharmaceutical dosage forms which contain a compound of the invention. The scope of the invention in its pharmaceutical composition aspect is not to be limited by the examples provided.

Pharmaceutical Dosage Form Examples

EXAMPLE A-Tablets

| No. | Ingredients | mg/tablet | mg/tablet |
|---|---|---|---|
| 1. | Active compound | 100 | 500 |
| 2. | Lactose USP | 122 | 113 |
| 3. | Corn Starch, Food Grade, as a | 30 | 40 |
| | 10% paste in Purified Water | | |
| 4. | Corn Starch, Food Grade | 45 | 40 |
| 5. | Magnesium Stearate | 3 | 7 |
| | Total | 300 | 700 |

Method of Manufacture

Mix Item Nos. 1 and 2 in a suitable mixer for 10-15 minutes. Granulate the mixture with Item No. 3. Mill the damp granules through a coarse screen (e.g., ¼″, 0.63 cm) if necessary. Dry the damp granules. Screen the dried granules if necessary and mix with Item No. 4 and mix for 10-15 minutes. Add Item No. 5 and mix for 1-3 minutes. Compress the mixture to appropriate size and weigh on a suitable tablet machine.

EXAMPLE B-Capsules

| No. | Ingredient | mg/capsule | mg/capsule |
|---|---|---|---|
| 1. | Active compound | 100 | 500 |
| 2. | Lactose USP | 106 | 123 |
| 3. | Corn Starch, Food Grade | 40 | 70 |
| 4. | Magnesium Stearate NF | 7 | 7 |
| | Total | 253 | 700 |

Method of Manufacture

Mix Item Nos. 1, 2 and 3 in a suitable blender for 10-15 minutes. Add Item No. 4 and mix for 1-3 minutes. Fill the mixture into suitable two-piece hard gelatin capsules on a suitable encapsulating machine.

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

1. A method of treating cough comprising administering to a mammal in need of such treatment an effective amount of an ORL-1 agonist, alone or in combination with an effective amount of one or more agents for treating cough, allergy or asthma symptoms.

2. The method of claim 1 wherein the ORL-1 agonist is selected from the group consisting of:
a) a compound represented by the structural formula IA: 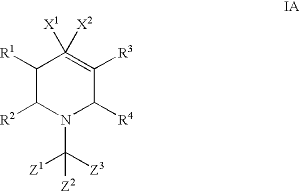
or a pharmaceutically acceptable salt or solvate thereof, wherein:
the dotted line represents an optional double bond;
X¹ is R⁵—(C₁-C₁₂)alkyl, R⁶—(C₃-C₁₂)cycloalkyl, R⁷-aryl, R⁸-heteroaryl or R¹⁰—(C₃-C₇)heterocycloalkyl;
X² is —CHO, —CN, —NHC(═NR²⁶)NHR²⁶, —CH(═NOR²⁶), —NHOR²⁶, R⁷-aryl, R⁷-aryl(C₁-C₆)alkyl, R⁷-aryl(C₁-C₆)alkenyl, R⁷-aryl(C₁-C₆)-alkynyl, —(CH₂)ᵥOR¹³, —(CH₂)ᵥCOOR²⁷, —(CH₂)ᵥCONR¹⁴R¹⁵, —(CH₂)ᵥNR²¹R²² or —(CH₂)ᵥNHC(O)R²¹, wherein v is zero, 1, 2 or 3 and wherein q is 1 to 3 and a is 1 or 2;
or X¹ is 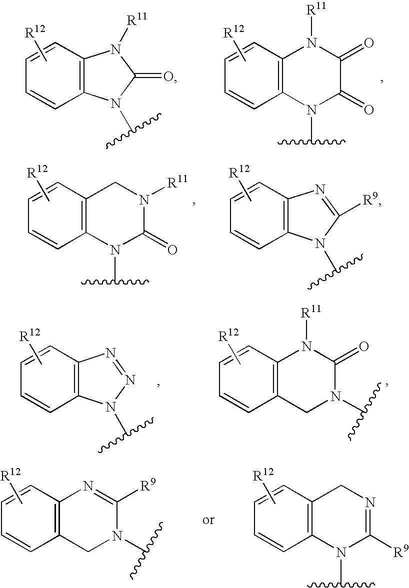
and X² is hydrogen;
or X¹ and X² together form a spiro group of the formula 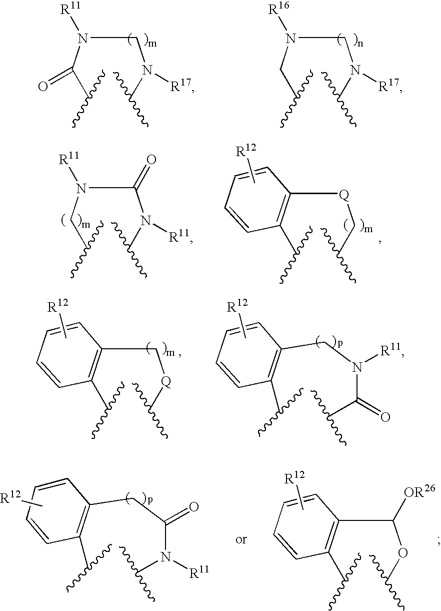
is 1 or 2;
n is 1, 2 or 3, provided that when n is 1, one of R¹⁶ and R¹⁷ is —C(O)R²⁸;
p is 0 or 1;
Q is —CH₂—, —O—, —S—, —SO—, —SO₂— or —NR¹⁷—;
R¹, R², R³ and R⁴ are independently selected from the group consisting of hydrogen and (C₁-C₆)alkyl, or (R¹ and R⁴) or (R² and R³) or (R¹ and R³) or (R² and R⁴) together can form an alkylene bridge of 1 to 3 carbon atoms;
R⁵ is 1 to 3 substituents independently selected from the group consisting of H, R⁷-aryl, R⁶—(C₃-C₁₂)cycloalkyl, R⁸-heteroaryl, R¹⁰—(C₃-C₇)heterocycloalkyl, —NR¹⁹R²⁰, —OR¹³ and —S(O)₀₋₂R¹³;
R⁶ is 1 to 3 substituents independently selected from the group consisting of H, (C₁-C₆)alkyl, R⁷-aryl, —NR¹⁹R²⁰, —OR¹³ and —SR¹³;
R⁷ is 1 to 3 substituents independently selected from the group consisting of hydrogen, halo, (C₁-C₆)alkyl, R²⁵-aryl, (C₃-C₁₂)cycloalkyl, —CN, —CF₃, —OR¹⁹, —(C₁-C₆)alkyl-OR¹⁹, —OCF₃, —NR¹⁹R²⁰, —(C₁-C₆)alkyl-NR¹⁹R²⁰, —NHSO₂R¹⁹, —SO₂N(R²⁶)₂, —SO₂R¹⁹, —SOR¹⁹, —SR¹⁹, —NO₂, —CONR¹⁹R²⁰, —NR²⁰COR¹⁹, —COR¹⁹, —COCF₃, —OCOR¹⁹, —OCO₂R¹⁹, —COOR¹⁹, —(C₁-C₆)alkyl-NHCOOC(CH₃)₃, —(C₁-C₆)alkyl-NHCOCF₃, —(C₁-C₆)alkyl-NHSO₂—(C₁-C₆)alkyl, —(C₁-C₆)alkyl-NHCONH—(C₁-C₆)-alkyl or 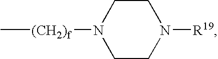
wherein f is 0 to 6; or R⁷ substituents on adjacent ring carbon atoms may together form a methylenedioxy or ethylenedioxy ring;
R⁸ is 1 to 3 substituents independently selected from the group consisting of hydrogen, halo, (C₁-C₆)alkyl, R²⁵-aryl, (C₃-C₁₂)cycloalkyl, —CN, —CF₃, —OR¹⁹, —(C₁-C₆)alkyl-OR¹⁹, —OCF₃, —NR¹⁹R²⁰, —(C₁-C₆)alkyl-NR¹⁹R²⁰, —NHSO₂R¹⁹, —SO₂N(R²⁶)₂, —NO₂, —CONR¹⁹R²⁰, —NR²⁰COR¹⁹, —COR¹⁹, —OCOR¹⁹, —OCO₂R¹⁹ and —COOR^{19;}
R⁹ is hydrogen, (C₁-C₆)alkyl, halo, —OR¹⁹, —NR¹⁹R²⁰, —NHCN, —SR¹⁹ or —(C₁-C₆)alkyl-NR¹⁹R²⁰;
R¹⁰ is H, (C₁-C₆)alkyl, —OR¹⁹, —(C₁-C₆)alkyl-OR¹⁹, —NR¹⁹R²⁰ or —(C₁-C₆)alkyl-NR¹⁹R²⁰;
R¹¹ is independently selected from the group consisting of H, R⁵—(C₁-C₆)alkyl, R⁶—(C₃-C₁₂)cycloalkyl, —(C₁-C₆)alkyl(C₃-C₁₂)cycloalkyl, —(C₁-C₆)alkyl-OR¹⁹, —(C₁-C₆)alkyl-NR¹⁹R²⁰ and 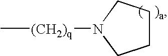
wherein q and a are as defined above;
R¹² is H, (C₁-C₆)alkyl, halo, —NO₂, —CF₃, —OCF₃, —OR¹⁹, —(C₁-C₆)alkyl-OR¹⁹, —NR¹⁹R²⁰ or —(C₁-C₆)alkyl-NR¹⁹R²⁰;
R¹³ is H, (C₁-C₆)alkyl, R⁷-aryl, —(C₁-C₆)alkyl-OR¹⁹, —(C₁-C₆)alkyl-NR¹⁹R²⁰; —(C₁-C₆)alkyl-SR¹⁹; or aryl (C₁-C₆) alkyl;
R¹⁴ and R¹⁵ are independently selected from the group consisting of H, R⁵—(C₁-C₆)alkyl, R⁷-aryl and 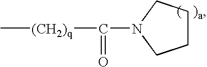
wherein q and a are as defined above;
R¹⁶ and R¹⁷ are independently selected from the group consisting of hydrogen, R⁵—(C₁-C₆)alkyl, R⁷-aryl, (C₃-C₁₂)cycloalkyl, R⁸-heteroaryl, R⁸-heteroaryl(C₁-C₆)alkyl, —C(O)R²⁸, —(C₁-C₆)alkyl(C₃-C₇)-heterocycloalkyl, —(C₁-C₆)alkyl-OR¹⁹ and —(C₁-C₆)alkyl-SR¹⁹;
R¹⁹ and R²⁰ are independently selected from the group consisting of hydrogen, (C₁-C₆)alkyl, (C₃-C₁₂)cycloalkyl, aryl and aryl(C₁-C₆)alkyl;
R²¹ and R²² are independently selected from the group consisting of hydrogen, (C₁-C₆)alkyl, (C₃-C₁₂)cycloalkyl, (C₃-C₁2)cycloalkyl(C₁-C₆)alkyl, (C₃-C₇)heterocycloalkyl, —(C₁-C₆)alkyl(C₃-C₇)-heterocycloalkyl, R⁷-aryl, R⁷-aryl(C₁-C₆)alkyl, R⁸-heteroaryl(C₁-C₁₂)alkyl, —(C₁-C₆)alkyl-OR¹⁹, —(C₁-C₆)alkyl-NR¹⁹R²⁰, —(C₁-C₆)alkyl-SR¹⁹, —(C₁-C₆)alkyl-NR¹⁸—(C₁-C₆)alkyl-O—(C₁-C₆)alkyl-NR¹⁸—(C₁-C₆)alkyl-NR¹⁸—(C₁-C₆)alkyl;
R¹⁸ is hydrogen or (C₁-C₆)alkyl;
Z¹ is R⁵—(C₁-C₁₂)alkyl, R⁷-aryl, R⁸-heteroaryl, R⁶—(C₃-C₁₂)cyclo-alkyl, R¹⁰—(C₃-C₇)heterocycloalkyl, —CO₂(C₁-C₆)alkyl, CN or —C(O)NR¹⁹R²⁰; Z² is hydrogen or Z¹; Z³ is hydrogen or (C₁-C₆)alkyl; or Z¹, Z² and Z³, together with the carbon to which they are attached, form the group 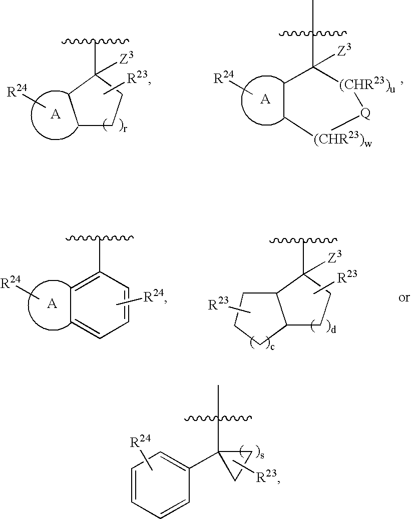
wherein r is 0 to 3; w and u are each 0-3, provided that the sum of w and u is 1-3; c and d are independently 1 or 2; s is 1 to 5; and ring A is a fused R⁷-phenyl or R⁸-heteroaryl ring;
R²³ is 1 to 3 substituents independently selected from the group consisting of H, (C₁-C₆)alkyl, —OR¹⁹, —(C₁-C₆)alkyl-OR¹⁹, —NR¹⁹R²⁰ and —(C₁-C₆)alkyl-NR¹⁹R²⁰;
R²⁴ is 1 to 3 substituents independently selected from the group consisting of R²³, —CF₃, —OCF₃, NO₂ or halo, or R²⁴ substituents on adjacent ring carbon atoms may together form a methylenedioxy or ethylenedioxy ring;
R²⁵ is 1-3 substituents independently selected from the group consisting of H, (C₁-C₆)alkyl, (C₁-C₆)alkoxy and halo;
R²⁶ is independently selected from the group consisting of H, (C₁-C₆)alkyl and R²⁵—C₆H₄—CH₂—;
R²⁷ is H, (C₁-C₆)alkyl, R⁷-aryl(C₁-C₆)alkyl, or (C₃-C₁₂)cycloalkyl; and
R²⁸ is (C₁-C₆)alkyl, —(C₁-C₆)alkyl(C₃-C₁₂)cycloalkyl, R⁷-aryl, R⁷-aryl-(C₁-C₆)alkyl, R⁸-heteroaryl, —(C₁-C₆)alkyl-NR¹⁹R²⁰, —(C₁-C₆)alkyl-OR¹⁹ or —(C₁-C₆)alkyl-SR¹⁹;
b) a compound represented by the structural formula II: 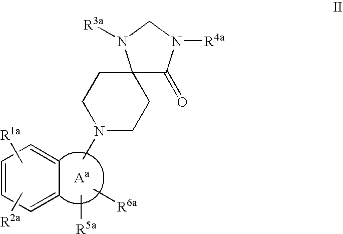
wherein
R^{1a} and R^{2a} are, independently from each other, hydrogen, lower alkyl, lower alkoxy or halogen;
R^{3a} is phenyl, optionally substituted by lower alkyl, CF₃, lower alkoxy or halogen; and
R^{4a} is hydrogen, lower alkyl, lower alkenyl, —C(O)-lower alkyl, —C(O)-phenyl, lower alkyl-C(O)-phenyl, lower alkylen-C(O)-lower alkyl, lower alkantriyl-di-C(O)O-lower alkyl, hydroxy-lower alkyl, lower alkyl-O-lower alkyl, lower alkyl-CH(OH)CF₃, phenyl or benzyl;
R^{5a} and R^{6a} are, independently from each other, hydrogen, phenyl, lower alkyl or di-lower alkyl or may form together a phenyl ring, and
R^{5a} and one of R^{1a} or R^{2a} may form together a saturated or unsaturated 6 membered ring,
A^{a} is a 4-7 membered saturated ring which may contain a heteroatom such as O or S,
or a pharmaceutically acceptable acid addition salt thereof;
c) a compound represented by the structural formula III 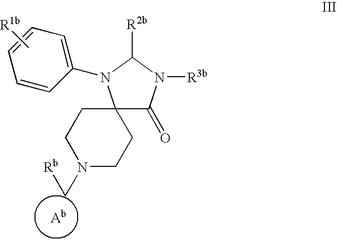
wherein
R^{1b} is hydrogen, lower alkyl, halogen, lower alkoxy, CF₃, lower alkyl-phenyl or (C₅₋₇)-cycloalkyl;
R^{2b} is hydrogen, lower alkyl, phenyl or lower alkyl-phenyl;
R^{3b} is hydrogen, lower alkyl, benzyl, lower alkyl-phenyl, lower alkyl-diphenyl, triazinyl, cyanomethyl, lower alkyl-piperidinyl, lower alkyl-naphthyl, (C₅₋₇)-cycloalkyl, lower alkyl-(C₅₋₇)-cycloalkyl, lower alkyl-pyridyl, lower alkyl-morpholinyl, lower alkyl dioxolanyl, lower alkyl, oxazolyl, or lower alkyl-2-oxo-oxazolidinyl and wherein the ring systems may be substituted by additional lower alkyl, lower alkoxy, CF₃ or phenyl, or —(CH₂)ₙC(O)O-lower alkyl, —(CH₂)ₙC(O)NH₂, —(CH₂)ₙC(O)N(lower alkyl)₂, —(CH₂)ₙOH or —(CH₂)ₙC(O)NHCH₂C₆H₆;
R^{4b} is hydrogen, lower alkyl or nitrilo;
A^{b} is a ring system, consisting of
(a) (C₅⁻₁₅)-cycloalkyl, which may be in addition to R^{4b} optionally substituted by lower alkyl, CF₃, phenyl, (C₅⁻₇)-cycloalkyl, spiro-undecan-alkyl or by 2-norbornyl, or is one of the following groups 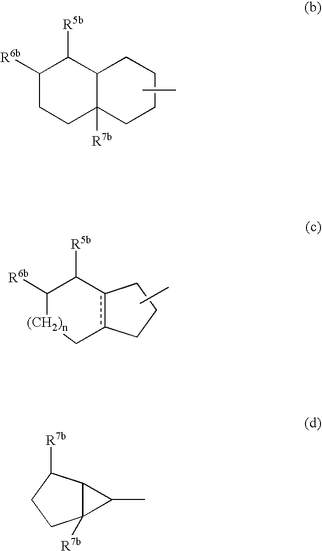
dodecahydro-acenaphthylen-1yl (e), bicyclo[6.2.0]dec-9-yl (f) and bicyclononan-9-yl (g); and wherein
R^{5b} and R^{6b} are hydrogen, lower alkyl, or taken together and with the carbon atoms to which they are attached form a phenyl ring;
R^{7b} is hydrogen or lower alkyl;
the dotted line represents an optional double bond and n is 1 to 4;
or a pharmaceutically acceptable acid addition salt thereof;
d) a compound represented by the structural formula IV: 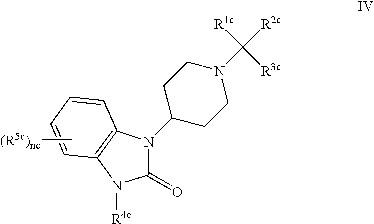
or a pharmaceutically acceptable salt thereof, wherein
R^{1c} and R^{2c} are independently C₁-C₄ alkyl; or
R^{1c} and R^{2c}, taken together with the carbon to which they are attached, form a mono-, bi-, tri- or spiro-cyclic group having 6 to 13 carbon atoms, wherein the cyclic group is optionally substituted by 1 to 5 substituents independently selected from C₁-C₄ alkyl, C₂-C₄ alkylene, C₁-C₄ alkoxy, hydroxy, oxo, ═CH₂ and ═CH—C₁-C₄ alkyl;
R^{3c} is C₁-C₇ alkyl, C₂-C₅ alkenyl, C₂-C₅ alkynyl, phenyl-C₁-C₅ alkyl, phenyl optionally substituted by 1 to 3 substituents independently selected from fluorine, C₁-C₃ alkyl and C₁-C₃ alkoxy, or a heteroaryl group selected from furyl, theinyl, pyrrolyl and pyridyl, wherein said heteroaryl group is optionally substituted by 1 to 3 substituents independently selected from halo, C₁-C₃ alkyl and C₁-C₃ alkoxy, with the proviso that when both R^{1c} and R^{2c} are C₁-C₄ alkyl, then R^{3c} is other than C₁-C₇ alkyl, C₂-C₅ alkenyl and C₂-C₅ alkynyl;
R^{4c} is selected from
1) hydrogen;
2) optionally substituted mono- or di-substituted C₁-C₈ alkyl, C₃-C₇ cycloalkyl, C₂-C₈ alkenyl, C₂-C₈ alkynyl, C₁C₆ alkyl-Z^{c}-, C₁C₆ alkyl-Z^{c}-(C₁C₆)alkyl, C₃-C₇ cycloalkyl-Z^{c}-(C₁C₆)alkyl, C₂-C₆ alkenyl-Z^{c}-(C₁C₆)alkyl or C₂-C₆ alkynyl-Z^{c}-C₁C₆)alkyl, wherein Z^{c} is selected from O, S, SO, SO₂, CO, CO₂, OCO, NR^{c}, CONR^{c} and NR^{c}CO, wherein R^{c} is hydrogen or C₁-C₆ alkyl, and the substituents to be attached to the alkyl, alkenyl, alkynyl or cycloalkyl moiety are independently selected from halo, hydroxy, carboxy, amino, mono- or di-(C₁-C₄ alkyl)amino, hydrazino, azido, ureido, amidino and guanidino; or
3) optionally mono- or di-substituted aryl, heterocyclic, aryl(C₁-C₅)alkyl, heterocyclic(C₁-C₅)alkyl, heterocyclic-heterocyclic(C₁-C₅)alkyl, aryl-heterocyclic(C₁-C₅)alkyl, heterocyclic-Z^{c}(C₁-C₅)alkyl, aryl-Z^{c}-(C₁-C₅)alkyl, aryl(C₁-C₅)alkyl-Z^{c}-(C₁-C₅)alkyl, or heterocyclic(C₁-C₅)alkyl-Z^{c}-(C₁-C₅)alkyl, wherein Z^{c} is selected from O, S, SO, SO₂, CO, CO₂, OCO, NR^{c}, CONR^{c} and NR^{c}CO, wherein R^{c} is hydrogen or C₁-C₆ alkyl, and the substituents to be attached to the aryl or heterocyclic moiety are independently selected from halo, hydroxy, carboxy, C₁-C₄ alkyl, C₁-C₄ alkoxy, C₁-C₄ alkyl-CO—, amino(C₁-C₄)alkyl-CO—, phenyl, benzyl, amino, mono- or di-(C₁-C₄ alkyl)amino, hydrazino, azido, ureido, amidino and guanidino;
R^{5c} is independently selected from halo, C₁-C₃ alkyl, C₁-C₃ alkoxy, C₁-C₃ alkylsulfonyl, CF₃, carboxy, hydroxy, amino, alkylamino, acylamino, arylcarbonyl, alkylcarbonyl and hydroxyalkyl; and
n is 0, 1, 2, 3 or4;
e) a compound represented by the structural formula V 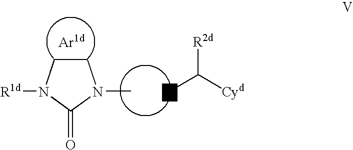
or a salt or ester thereof, wherein
Ar^{1d} is an optionally substituted aromatic carbon ring or heterocycle, wherein the optional substituents are independently selected from halo, alkyl, amino, alkylamino, dialkylamino, hydroxy, alkoxy and carboxyl; 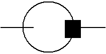
is an optionally substituted mono- or di-cyclic C₃₋₁₄ aliphatic nitrogenous heterocycle;
Cy^{d} is an optionally substituted mono-, di- or tri-cyclic C₃₋₂₀ aliphatic carbon ring;
R^{1d} is hydrogen, lower alkenyl, lower alkynyl, lower cycloalkyl, amino, lower alkylamino, di(lower alkyl)amino, hydroxy, lower alkoxy, carboxy, lower alkoxycarbonyl, carbamoyl, lower alkylcarbamoyl, di(lower alkyl)carbamoyl or optionally substituted lower alkyl; and
R^{2d} is hydrogen or lower alkyl; and
f) a compound represented by the structural formula VI 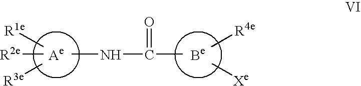
or a pharmaceutically acceptable salt thereof, wherein
A^{e} is an aryl or heterocyclyl ring;
B^{e} is phenyl, thienyl, furyl, pyrrolyl, pyrrolidinyl, oxazolyl or cyclohexenyl;
R^{1e} and R^{2e} are independently hydrogen, alkyl, hydroxyalkyl, amino, alkylamino or di-alkylamino;
R^{3e} and R^{4e} are independently hydrogen, halo or alkyl;
X^{e} is hydrogen, halo, alkyl, alkoxyalkyl, alkenyl, amino, CN, or —(CH₂)ₘₑ-E^{e}-(CH₂)ₙₑ-G^{e};
E^{e} is a bond, —CH═CR^{6e}, O, S, NR^{7e}, CO, SO₂ or NHCO;
G^{e} is aryl, heterocyclyl, cycloalkyl or fused aryl, all optionally substituted by 1-5 R^{5e} groups;
R^{5e} is independently selected from halo, OH, alkyl, alkyl optionally substituted by alkoxy, alkoxyalkoxy, halo, OH or alkanoyloxy, alkoxy, alkoxyalkoxy, amino, alkylamino, di-alkylamino, NO₂, CN, alkanoyl, alkanoyloxy, carboxy, alkoxycarbonyl, alkylsulfonyl and phenyl;
R^{6e} is hydrogen or aryl;
R^{7e} is hydrogen, alkyl or alkoxycarbonyl;
m^{e} is 0-8; and
n^{e} is 1-4.

3. The method of claim 2 wherein the compound is represented by structural formula IA.

4. The method of claim 3 wherein, in the compound of formula IA, Z¹ and Z² are each R⁷-aryl.

5. The method of claim 4, wherein R⁷ is selected from the group consisting of (C₁-C₆)alkyl and halo.

6. The method of claim 3 wherein, in the compound of formula IA, X¹ is R⁷-aryl and and X² is OH; or X² is hydrogen and X¹ is 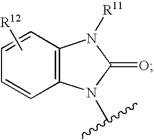
or X¹ and X² together form the spirocyclic group

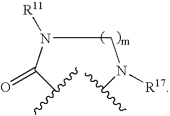

7. The method of claim 6 wherein X¹ is 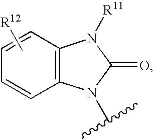
R¹² is hydrogen and R¹¹ is (C₁-C₆)alkyl, —(C₁-C₆) alkyl(C₃-C₁₂)cycloalkyl, —(C₁-C₆)alkyl-OR¹⁹ or —(C₁-C₆)alkyl-NR¹⁹R²⁰.

8. The method of claim 6 wherein X¹ and X² together form 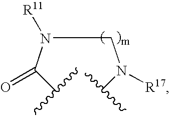
m is 1, R¹⁷ is phenyl and R¹¹ is —(C₁-C₆)alkyl-OR¹⁹ or —(C₁-C₆)alkyl-NR¹⁹R²⁰.

9. The method of claim 1 wherein the ORL-1 agonist is selected from

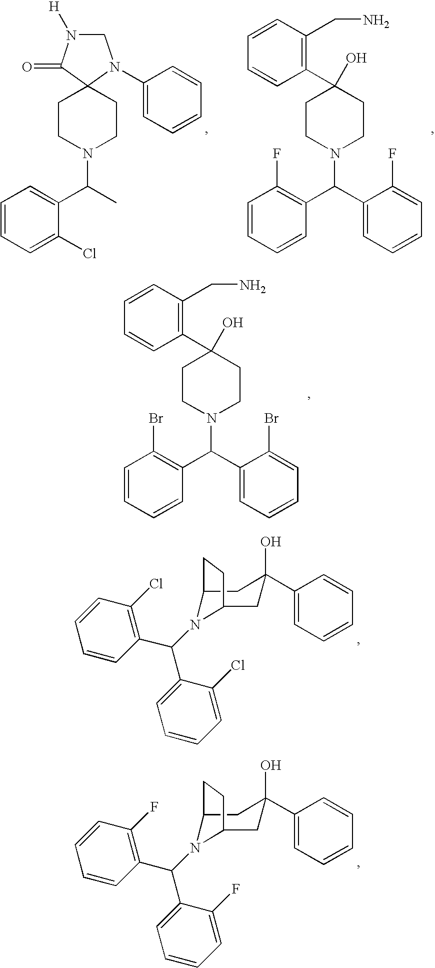

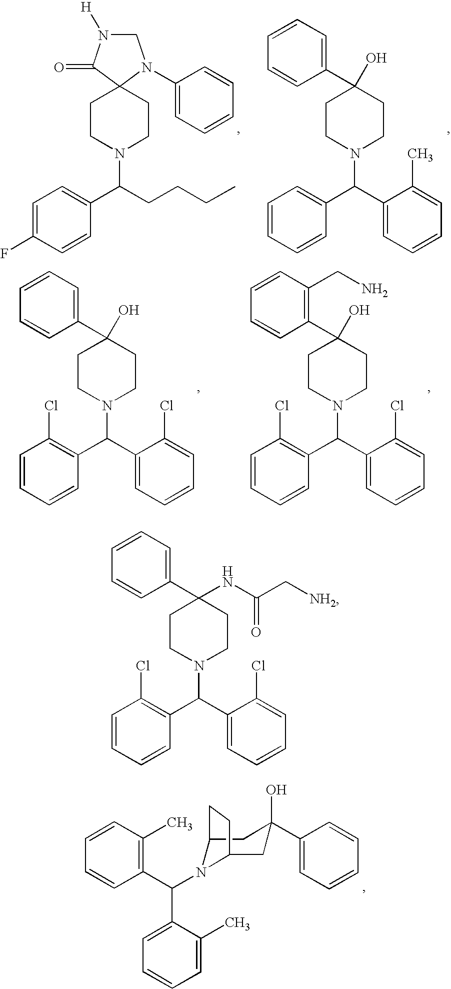

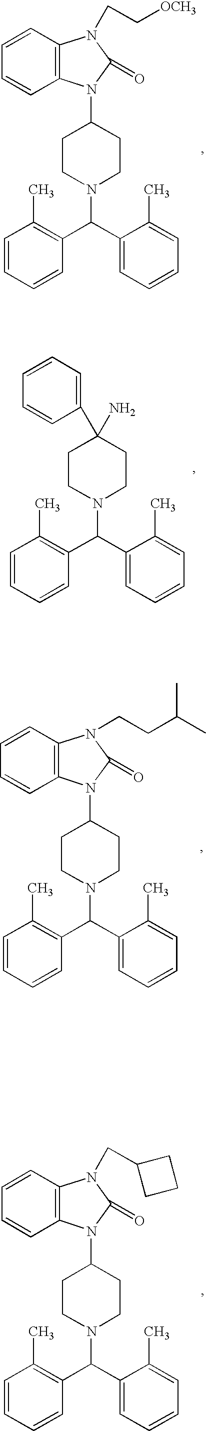

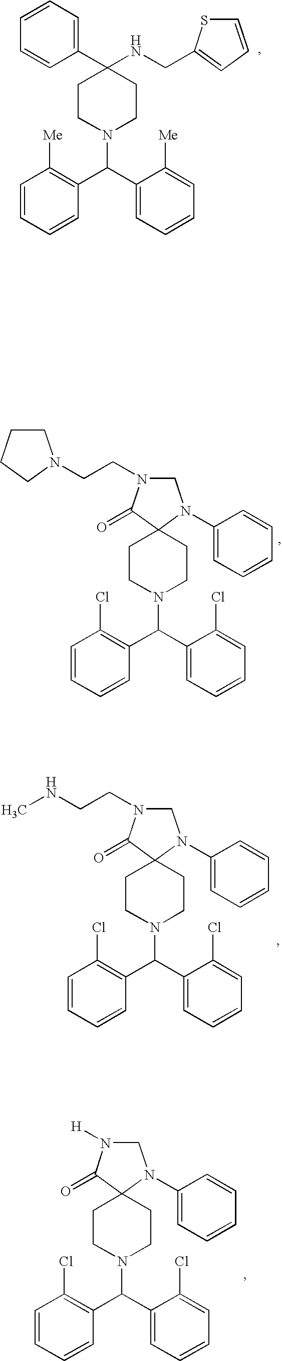

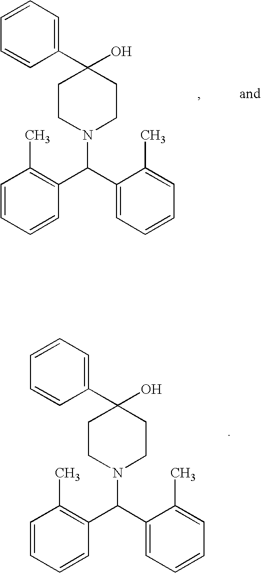

10. The method of claim 1 wherein the agents for treating cough, allergy or asthma symptoms are selected from the group consisting of: antihistamines, 5-lipoxygenase inhibitors, leukotriene inhibitors, H₃ inhibitors, β-adrenergic receptor agonists, xanthine derivatives, α-adrenergic receptor agonists, mast cell stabilizers, anti-tussives, expectorants, decongestants, NK₁, NK₂ and NK₃ tachykinin receptor antagonists, and GABA_{B} agonists.

11. A pharmaceutical composition comprising: a therapeutically effective amount of a nociceptin receptor ORL-1 agonist; a therapeutically effective amount of one or more agents selected from the group consisting of: antihistamines, 5-lipoxygenase inhibitors, leukotriene inhibitors, H₃ inhibitors, β-adrenergic receptor agonists, xanthine derivatives, α-adrenergic receptor agonists, mast cell stabilizers, anti-tussives, expectorants, decongestants, NK₁, NK₂ and NK₃ tachykinin receptor antagonists, and GABA_{B} agonists; and a pharmaceutically acceptable carrier.

12. A compound represented by the formula 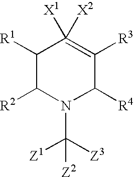
or a pharmaceutically acceptable salt or solvate thereof, wherein:
the dotted line represents an optional double bond;
X¹ is R⁵—(C₁-C₁₂)alkyl, R⁶—(C₃-C₁₂)cycloalkyl, R⁷-aryl, R⁸-heteroaryl or R¹⁰—(C₃-C₇)heterocycloalkyl;
X² is —CHO, —CN, —NHC(═NR²⁶)NHR²⁶, —CH(═NOR²⁶), —NHOR²⁶, R⁷-aryl, R⁷-aryl(C₁-C₆)alkyl, R⁷-aryl(C₁-C₆)alkenyl, R⁷-aryl(C₁-C₆)-alkynyl, —(CH₂)ᵥOR¹³, —(CH₂)ᵥCOOR²⁷, —(CH₂)ᵥCONR¹⁴R¹⁵, —(CH₂)ᵥNR²¹ R²² or —(CH₂)ᵥNHC(O)R²¹, wherein v is zero, 1, 2 or 3 and wherein q is 1 to 3 and a is 1 or 2;
or X¹ is 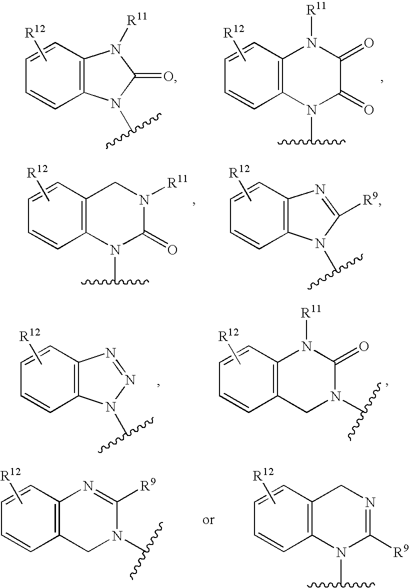
and X² is hydrogen;
or X¹ and X² together form a spiro group of the formula 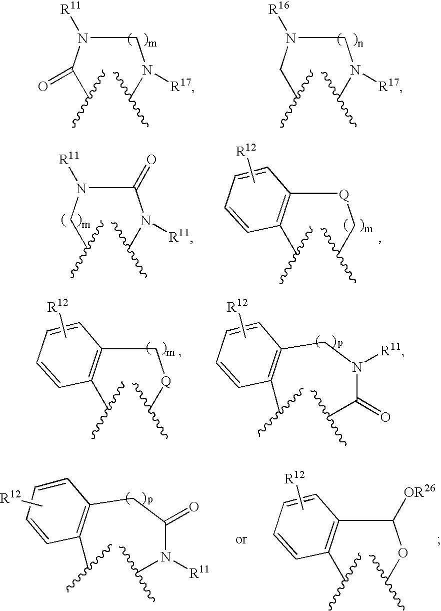
m is 1 or2;
n is 1, 2 or 3, provided that when n is 1, one of R¹⁶ and R¹⁷ is —C(O)R^{28;}
p is 0 or 1 ;
Q is —CH₂—, —O—, —S—, —SO—, —SO₂— or —NR¹⁷—;
R¹, R², R³ and R⁴ are independently selected from the group consisting of hydrogen and (C₁-C₆)alkyl, or (R¹ and R⁴) or (R² and R³) or (R¹ and R³) or (R² and R⁴) together can form an alkylene bridge of 1 to 3 carbon atoms;
R⁵ is 1 to 3 substituents independently selected from the group consisting of H, R⁷-aryl, R⁶—(C₃-C₁₂)cycloalkyl, R⁸-heteroaryl, R¹⁰—(C₃-C₇)heterocycloalkyl, —NR¹⁹R²⁰, —OR¹³ and —S(O)₀₋₂R¹³;
R⁶ is 1 to 3 substituents independently selected from the group consisting of H, (C₁-C₆)alkyl, R⁷-aryl, —NR¹⁹R²⁰, —OR¹³ and —SR¹³;
R⁷ is 1 to 3 substituents independently selected from the group consisting of hydrogen, halo, (C₁-C₆)alkyl, R²⁵-aryl, (C₃-C₁₂)cycloalkyl, —CN, —CF₃, —OR¹⁹, —(C₁-C₆)alkyl-OR¹⁹, —OCF₃, —NR¹⁹R²⁰, —(C₁-C₆)alkyl-NR¹⁹R²⁰, —NHSO₂R¹⁹, —SO₂N(R²⁶)₂, —SO₂R¹⁹, —SOR¹⁹, —SR¹⁹, —NO₂, —CONR¹⁹R²⁰, —NR²⁰COR¹⁹, —COR¹⁹, —COCF₃, —OCOR¹⁹, —OCO₂R¹⁹, —COOR¹⁹, —(C₁-C₆)alkyl-NHCOOC(CH₃)₃, —(C₁-C₆)alkyl-NHCOCF₃, —(C₁-C₆)alkyl-NHSO₂—(C₁-C₆)alkyl, —(C₁-C₆)alkyl-NHCONH—(C₁-C₆)-alkyl or 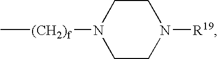
wherein f is 0 to 6; or R⁷ substituents on adjacent ring carbon atoms may together form a methylenedioxy or ethylenedioxy ring;
R⁸ is 1 to 3 substituents independently selected from the group consisting of hydrogen, halo, (C₁-C₆)alkyl, R²⁵-aryl, (C₃-C₁₂)cycloalkyl, —CN, —CF₃, —OR¹⁹, —(C₁-C₆)alkyl-OR¹⁹, —OCF₃, —NR¹⁹R²⁰, —(C₁-C₆)alkyl-NR¹⁹R²⁰, —NHSO₂R¹⁹, —SO₂N(R²⁶)₂, —NO₂, —CONR¹⁹R²⁰, —NR²⁰COR¹⁹, —COR¹⁹, —OCOR¹⁹, —OCO₂R¹⁹ and —COOR¹⁹;
R⁹ is hydrogen, (C₁-C₆)alkyl, halo, —OR¹⁹, —NR¹⁹R²⁰, —NHCN, —SR¹⁹ or —(C₁-C₆)alkyl-NR^{1 9}R²⁰;
R¹⁰ is H, (C₁-C₆)alkyl, —OR¹⁹, —(C₁-C₆)alkyl-OR¹⁹, —NR¹⁹R²⁰ or —(C₁-C₆)alkyl-NR¹⁹R²⁰;
R¹¹ is independently selected from the group consisting of H, R⁵—(C₁-C₆)alkyl, R⁶—(C₃-C₁₂)cycloalkyl, —(C₁-C₆)alkyl(C₃-C₁₂)cycloalkyl, —(C₁-C₆)alkyl-OR¹⁹, —(C₁-C₆)alkyl-NR¹⁹R²⁰ and 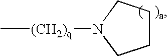
wherein q and a are as defined above;
R¹² is H, (C₁-C₆)alkyl, halo, —NO₂, —CF₃, —OCF₃, —OR¹⁹, —(C₁-C₆)alkyl-OR¹⁹, —NR¹⁹R²⁰ or —(C₁-C₆)alkyl-NR¹⁹R²⁰;
R¹³ is H, (C₁-C₆)alkyl, R⁷-aryl, —(C₁-C₆)alkyl-OR¹⁹, —(C₁-C₆)alkyl-NR¹⁹R²⁰ or —(C₁-C₆)alkyl-SR^{19;}
R¹⁴ and R¹⁵ are independently selected from the group consisting of H, R⁵—(C₁-C₆)alkyl, R⁷-aryl and 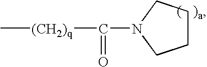
wherein q and a are as defined above;
R¹⁶ and R¹⁷ are independently selected from the group consisting of hydrogen, R⁵—(C₁-C₆)alkyl, R⁷-aryl, (C₃-C₁₂)cycloalkyl, R⁸-heteroaryl, R⁸-heteroaryl(C₁-C₆)alkyl, —C(O)R²⁸, —(C₁-C₆)alkyl(C₃-C₇)-heterocycloalkyl, —(C₁-C₆)alkyl-OR¹⁹ and —(C₁-C₆)alkyl-SR¹⁹;
R¹⁹ and R²⁰ are independently selected from the group consisting of hydrogen, (C₁-C₆)alkyl, (C₃-C₁₂)cycloalkyl, aryl and aryl(C₁-C₆)alkyl;
R²¹ and R²² are independently selected from the group consisting of hydrogen, (C₁-C₆)alkyl, (C₃-C₁₂)cycloalkyl, (C₃-C₁₂)cycloalkyl(C₁-C₆)alkyl, (C₃-C₇)heterocycloalkyl, —(C₁-C₆)alkyl(C₃-C₇)-heterocycloalkyl, R⁷-aryl, R⁷-aryl(C₁-C₆)alkyl, R⁸-heteroaryl(C₁-C₁₂)alkyl, —(C₁-C₆)alkyl-OR¹⁹, —(C₁-C₆)alkyl-NR¹⁹R²⁰, —(C₁-C₆)alkyl-SR¹⁹, —(C₁-C₆)alkyl-NR¹⁸—(C₁-C₆)alkyl-O—(C₁-C₆)alkyl-NR¹⁸—(C₁-C₆)alkyl-NR¹⁸—(C₁-C₆)alkyl;
R¹⁸ is hydrogen or (C₁-C₆)alkyl;
Z¹ is R⁵—(C₁-C₁₂)alkyl, R⁷-aryl, R⁸-heteroaryl, R⁶—(C₃-C₁₂)cyclo-alkyl, R¹⁰—(C₃-C₇)heterocycloalkyl, —CO₂(C₁-C₆)alkyl, CN or —C(O)NR¹⁹R²⁰; Z² is hydrogen or Z¹; Z³ is hydrogen or (C₁-C₆)alkyl; or Z¹, Z² and Z³, together with the carbon to which they are attached, form the group 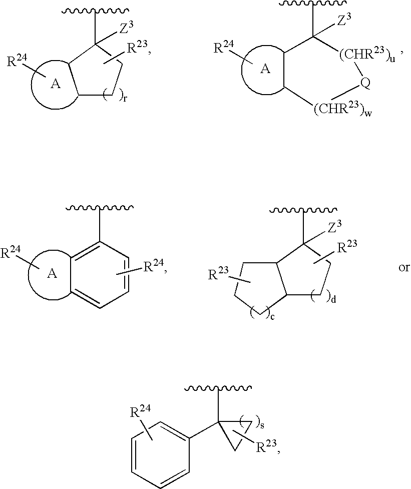
wherein r is 0 to 3; w and u are each 0-3, provided that the sum of w and u is 1-3; c and d are independently 1 or 2; s is 1 to 5; and ring A is a fused R⁷-phenyl or R⁸-heteroaryl ring;
R²³ is 1 to 3 substituents independently selected from the group consisting of H, (C₁-C₆)alkyl, —OR¹⁹, —(C₁-C₆)alkyl-OR¹⁹, —NR¹⁹R²⁰ and —(C₁-C₆)alkyl-NR¹⁹R²⁰;
R²⁴ is 1 to 3 substituents independently selected from the group consisting of R²³, —CF₃, —OCF₃, NO₂ or halo, or R²⁴ substituents on adjacent ring carbon atoms may together form a methylenedioxy or ethylenedioxy ring;
R²⁵ is 1-3 substituents independently selected from the group consisting of H, (C₁-C₆)alkyl, (C₁-C₆)alkoxy and halo;
R²⁶ is independently selected from the group consisting of H, (C₁-C₆)alkyl and R²⁵—C₆H₄—CH₂—;
R²⁷ is H, (C₁-C₆)alkyl, R⁷-aryl(C₁-C₆)alkyl, or (C₃-C₁₂)cycloalkyl;
R²⁸ is (C₁-C₆)alkyl, —(C₁-C₆)alkyl(C₃-C₁₂)cycloalkyl, R⁷-aryl, R⁷-aryl-(C₁-C₆)alkyl, R⁸-heteroaryl, —(C₁-C₆)alkyl-NR¹⁹R²⁰, —(C₁-C₆)alkyl-OR¹⁹ or —(C₁-C₆)alkyl-SR¹⁹; provided that when X¹ is 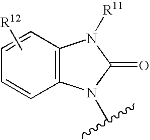
or X¹ and X² together are 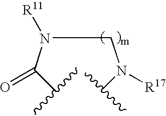
and Z¹ is R⁷-phenyl, Z² is not hydrogen or (C₁-C₃)alkyl;
provided that when Z¹, Z² and Z³, together with the carbon to which they are attached, form 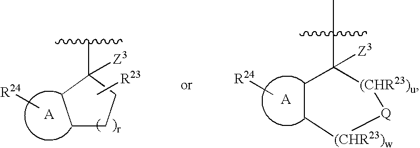
and X¹ and X² together are 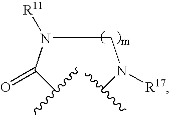
R¹¹ is not H, (C₁-C₆)alkyl, (C₁-C₆)alkoxy(C₁-C₆)alkyl or (C₁-C₆)hyd roxyalkyl;
provided that when R² and R⁴ form an alkylene bridge, Z¹, Z² and Z³, together with the carbon to which they are attached, are not 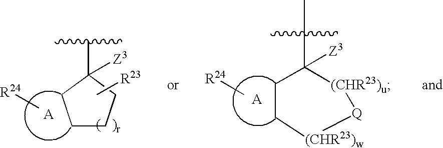
provided that when X¹ is 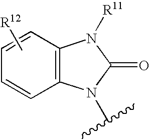
and Z¹ is R⁶—(C₃-C₁₂)-cycloalkyl, Z² is not H.

13. A compound of claim 12 wherein Z¹ is R⁷-aryl, Z² is C₄₋₁₂-alkyl and X¹ and X² form a spirocyclic group.

14. A compound of claim 13 wherein Z¹ is R⁷-phenyl, and X¹ and X² together form the spirocyclic group

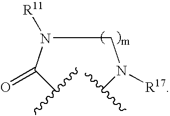

15. A compound of claim 14 wherein m is 1, R¹⁷ is phenyl and R¹¹ is —(C₁-C₆)alkyl-OR¹⁹ or —(C₁-C₆)alkyl-NR¹⁹R²⁰.

16. A pharmaceutical composition comprising a therapeutically effective amount of compound of claim 13 in combination with a pharmaceutically acceptable carrier.

17. A method of treating pain, anxiety, asthma, depression or alcohol abuse comprising administering an effective amount of a compound of claim 13 to a mammal in need of such treatment.

18. A compound of claim 13 selected from the group consisting of compounds represented by the formula 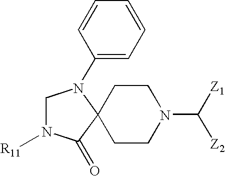
wherein R¹¹, Z¹ and Z² are as defined in the following table:

| R¹¹ | CH(Z¹)(Z²) |
|---|---|
| H | 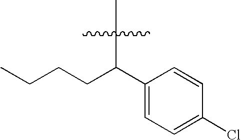 |
| H | 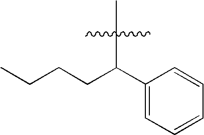 |
| H | 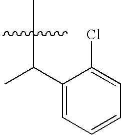 |
| H | 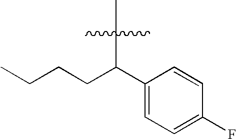 |
| H | 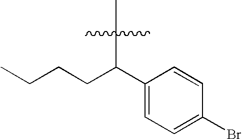 |
| H | 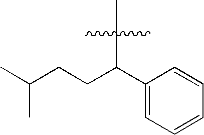 |
| 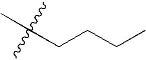 | 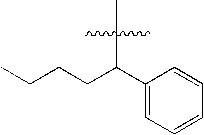 |
| 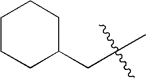 | 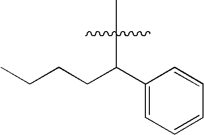 |
| 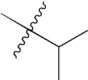 | 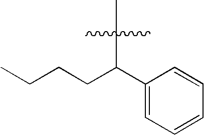 |
| 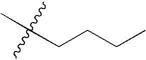 | 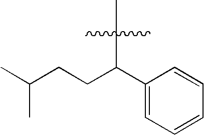 |
| 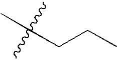 | 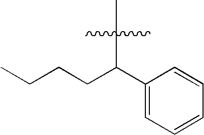 |
| 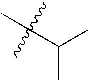 | 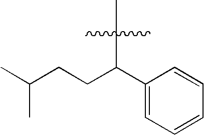 |
| H | 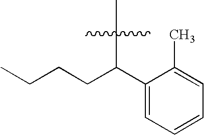 |
| 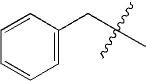 | 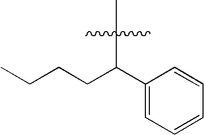 |
| 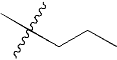 | 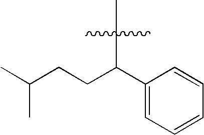 |
| 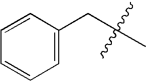 | 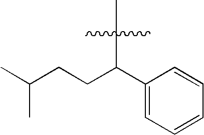 |
| 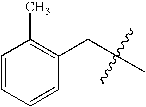 | 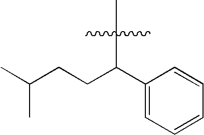 |
| 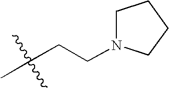 | 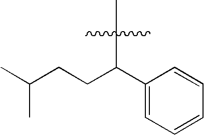 |
| 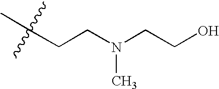 | 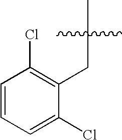 |
| 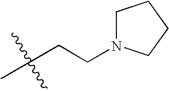 | 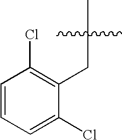 |
| 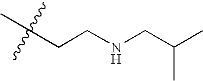 | 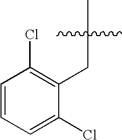 |
| 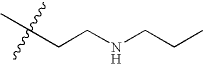 | 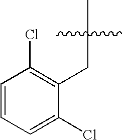 |
| 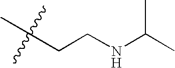 | 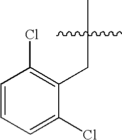 |
| 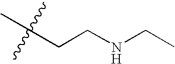 | 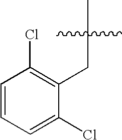 |
| 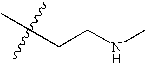 | 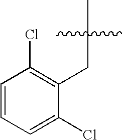 |
| 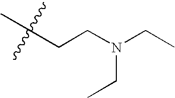 | 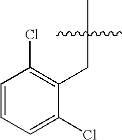 |
| 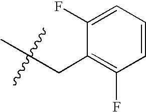 | 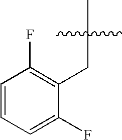 |
| 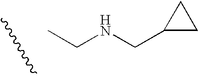 | 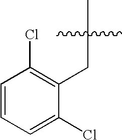 |
| 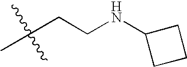 | 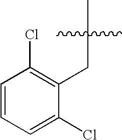 |
| H | 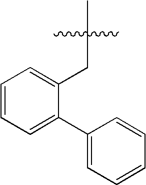 |
| 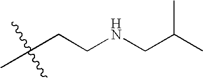 | 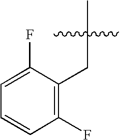 |
| 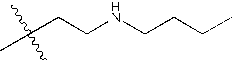 | 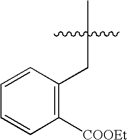 |
| 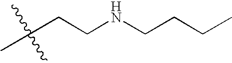 | 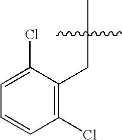 |
| 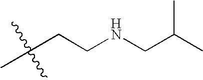 | 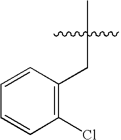 |
| 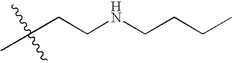 | 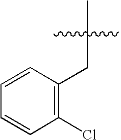 |
| 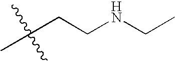 | 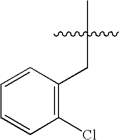 |
| 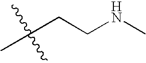 | 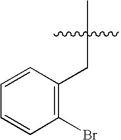 |
| 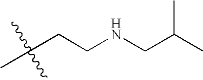 | 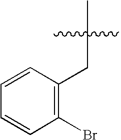 |
| 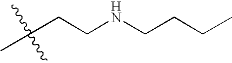 | 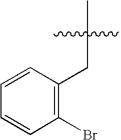 |
| 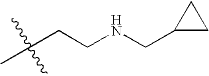 | 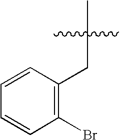 |
| 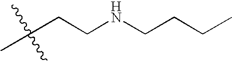 | 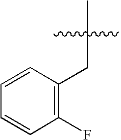 |
| 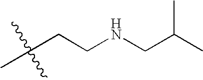 | 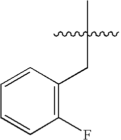 |
| H | 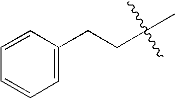 |
| 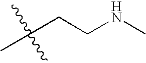 | 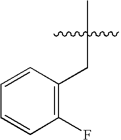 |
| 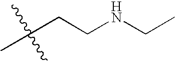 | 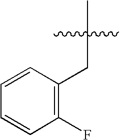 |

19. A compound selected from the group consisting of compounds represented by the formula 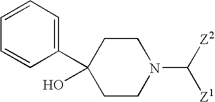
wherein Z¹ and Z² are as defined below:

| Z¹ | Z² |
|---|---|
| 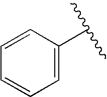 | 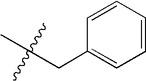 |
| 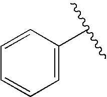 | 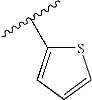 |
| 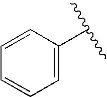 | 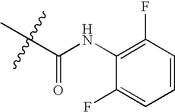 |
| 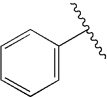 | 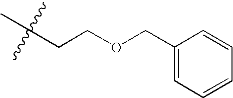 |
| Methyl | 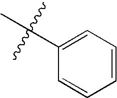 |
| Ethyl | 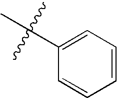 |
| 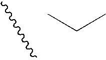 | 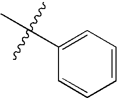 |
| 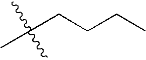 | 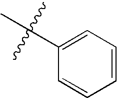 |
| 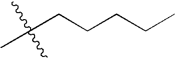 | 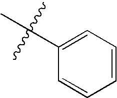 |
| 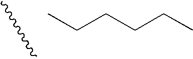 | 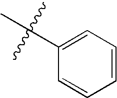 |
| 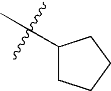 | 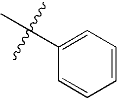 |
| 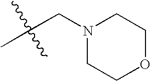 | 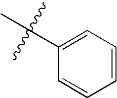 |
| 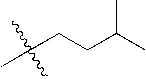 | 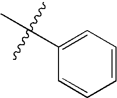 |

compounds represented by the formula 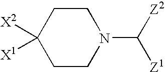
wherein X¹, X², Z¹ and Z² are as defined below

| X¹ | X² | Z¹ | Z² |
|---|---|---|---|
| 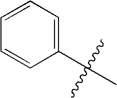 | NH₂ | 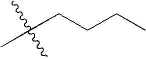 | 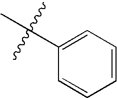 |
| 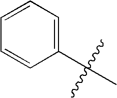 | 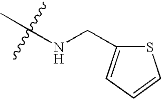 | 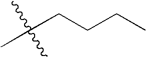 | 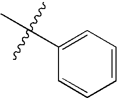 |
| 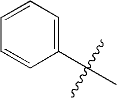 | CH₂NH₂ | 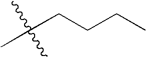 | 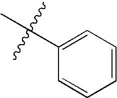 |
| 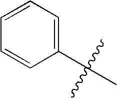 | 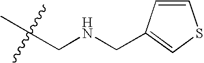 | 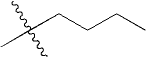 | 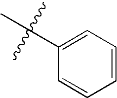 |
| 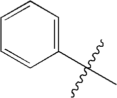 | 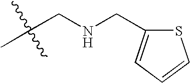 | 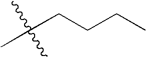 | 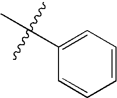 |
| 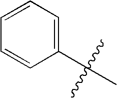 | 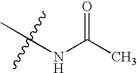 | 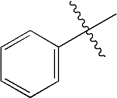 | 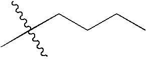 |
| 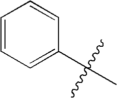 | OH | 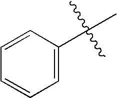 | 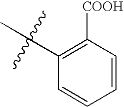 |
| 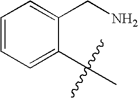 | OH | 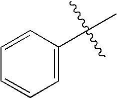 | —(CH₂)₄CH₃ |
| 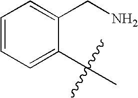 | OH | 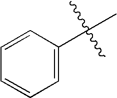 | —(CH₂)₃CH₃ |
| 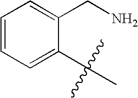 | OH | H | 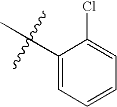 |
| 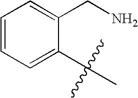 | OH | H | 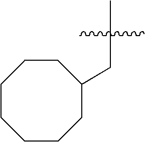 |
| 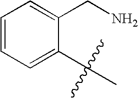 | OH | CH₃(CH₂)₃— | 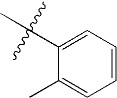 |
| 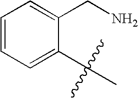 | OH | 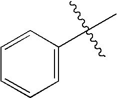 | 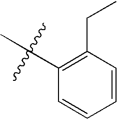 |

compounds represented by the formula 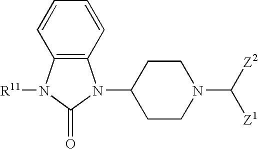
wherein R¹¹, Z¹ and Z² are as defined below,
wherein Ac is acetyl, Me is methyl and Et is ethyl:

| R¹¹ | CH(Z¹)(Z²) |
|---|---|
| H | 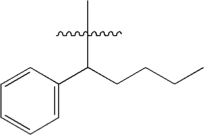 |
| 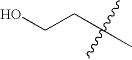 | 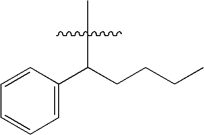 |
| H | 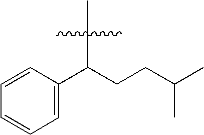 |
| 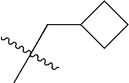 | 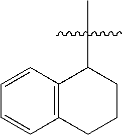 |
| 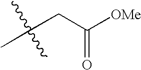 | 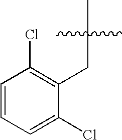 |
| H | 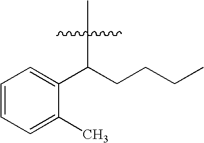 |
| 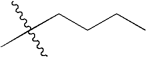 | 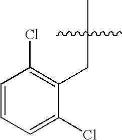 |
| 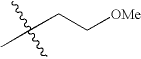 | 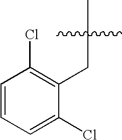 |
| 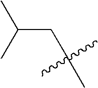 | 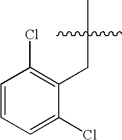 |
| 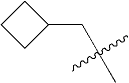 | 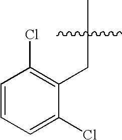 |
| 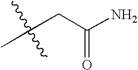 | 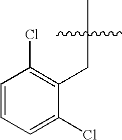 |

compounds represented by the formula 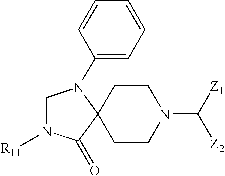
wherein R¹¹, Z¹ and Z² are as defined below:

| R¹¹ | CH(Z¹)(Z²) |
|---|---|
| H | 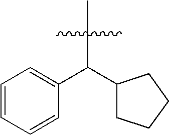 |
| H | 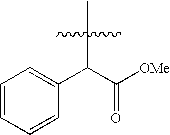 |
| H | 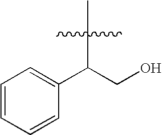 |
| H | 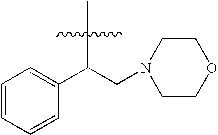 |
| 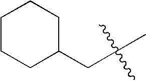 | 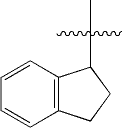 |
| 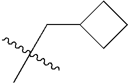 | 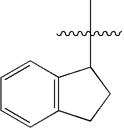 |
| 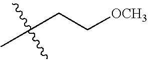 | 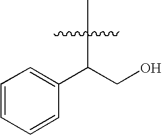 |
| 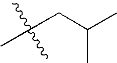 | 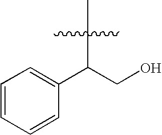 |
| 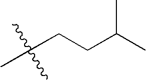 | 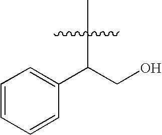 |
| 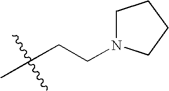 | 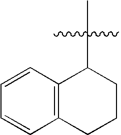 |
| 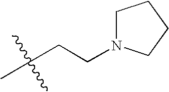 | 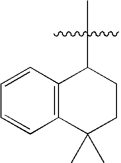 |
| 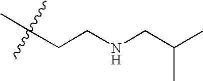 | 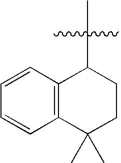 |
| 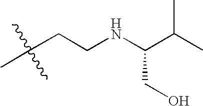 | 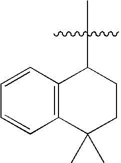 |
| 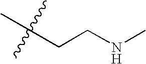 | 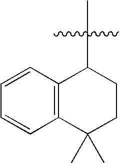 |
| 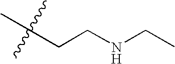 | 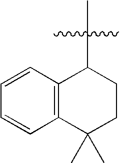 |
| 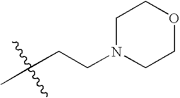 | 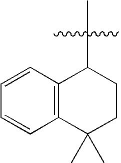 |
| 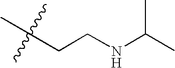 | 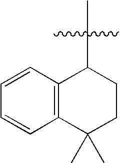 |
| 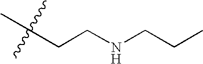 | 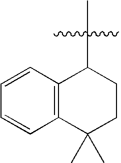 |
| 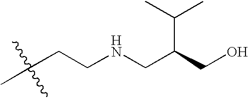 | 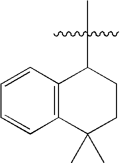 |
| 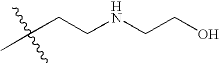 | 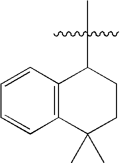 |
| 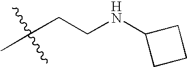 | 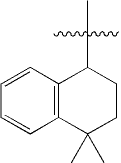 |
| 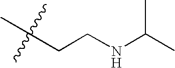 | 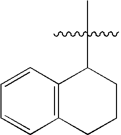 |
| 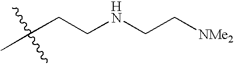 | 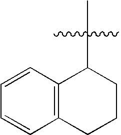 |
| 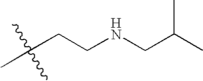 | 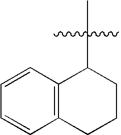 |
| 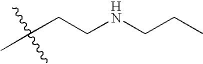 | 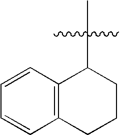 |
| 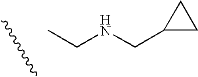 | 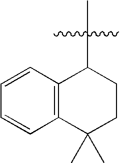 |
| 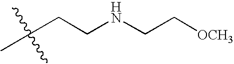 | 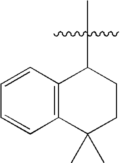 |
| 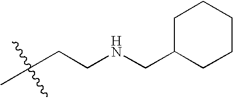 | 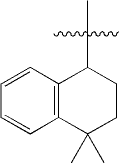 |
| 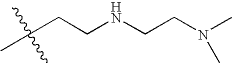 | 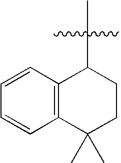 |
| 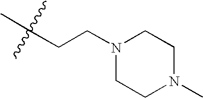 | 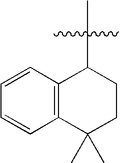 |
| 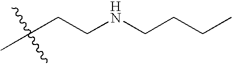 | 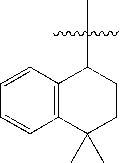 |
| 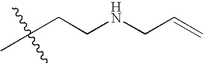 | 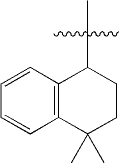 |
| 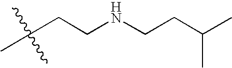 | 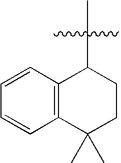 |
| 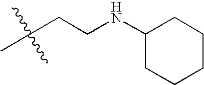 | 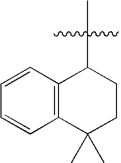 |
| 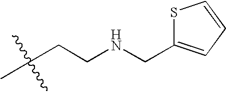 | 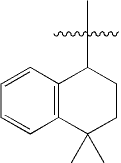 |
| 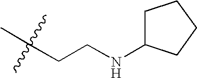 | 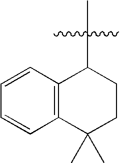 |
| 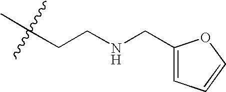 | 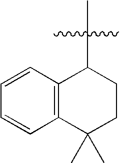 |
| 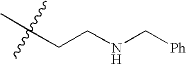 | 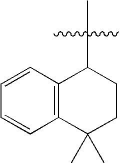 |
| 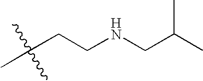 | 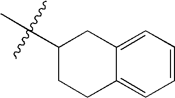 |
| 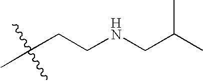 | 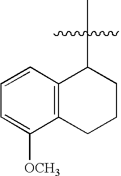 |
| 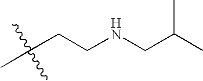 | 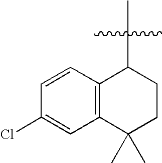 |
| 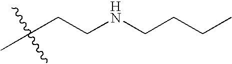 | 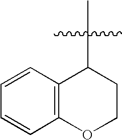 |
| 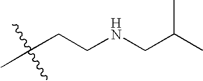 | 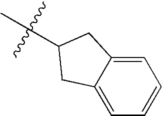 |
| H | 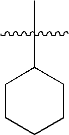 |
| 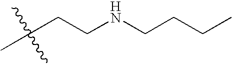 | 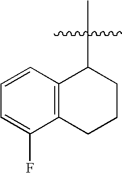 |
| 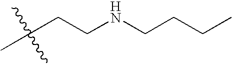 | 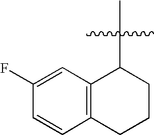 |
| 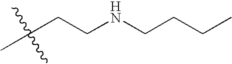 | 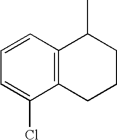 |
| H | 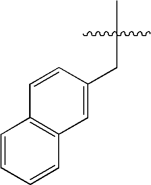 |
| 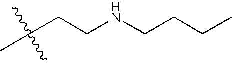 | 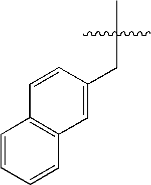 |
| H | 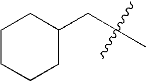 |
| H | 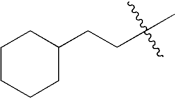 |
| H | 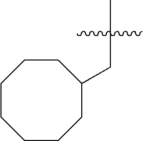 |
| 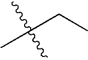 | 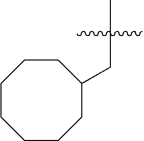 |

and compounds of the formulas shown below, wherein Me is methyl 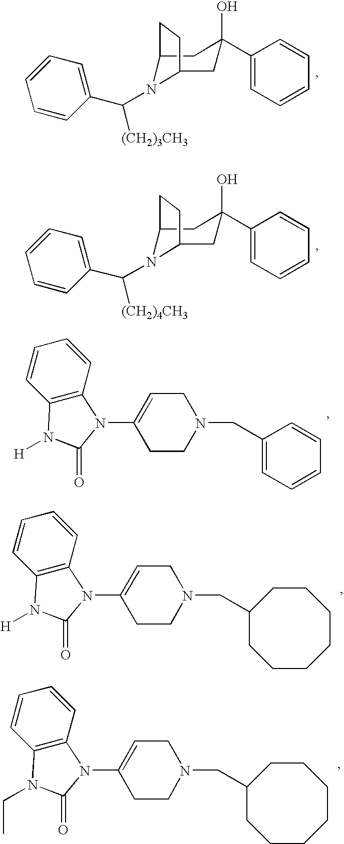 [chemical structure]
